SEQUENCE LISTING (26 sequences)

SEQ ID NO: 1 - DNA, 1650 bp, Escherichia coli
atgaaaaacatcaatccaacgcagaccgctgcctggcaggcactacagaaacacttcgatgaaatgaaagacgttacgatcgccgatctttttgctaaagacggcgatcgtttttctaagttctccgcaaccttcgacgatcagatgctggtggattactccaaaaaccgcatcactgaagagacgctggcgaaattacaggatctggcgaaagagtgcgatctggcgggcgcgattaagtcgatgttctctggcgagaagatcaaccgcactgaaaaccgcgccgtgctgcacgtagcgctgcgtaaccgtagcaataccccgattttggttgatggcaaagacgtaatgccggaagtcaacgcggtgctggagaagatgaaaaccttctcagaagcgattatttccggtgagtggaaaggttataccggcaaagcaatcactgacgtagtgaacatcgggatcggcggttctgacctcggcccatacatggtgaccgaagctctgcgtccgtacaaaaaccacctgaacatgcactttgtttctaacgtcgatgggactcacatcgcggaagtgctgaaaaaagtaaacccggaaaccacgctgttcttggtagcatctaaaaccttcaccactcaggaaactatgaccaacgcccatagcgcgcgtgactggttcctgaaagcggcaggtgatgaaaaacacgttgcaaaacactttgcggcgctttccaccaatgccaaagccgttggcgagtttggtattgatactgccaacatgttcgagttctgggactgggttggcggccgttactctttgtggtcagcgattggcctgtcgattgttctctccatcggctttgataacttcgttgaactgctttccggcgcacacgcgatggacaagcatttctccaccacgcctgccgagaaaaacctgcctgtactgctggcgctgattggcatctggtacaacaatttctttggtgcggaaactgaagcgattctgccgtatgaccagtatatgcaccgtttcgcggcgtacttccagcagggcaatatggagtccaacggtaagtatgttgaccgtaacggtaacgttgtggattaccagactggcccgattatctggggtgaaccaggcactaacggtcagcacgcgttctaccagctgatccaccagggaaccaaaatggtaccgtgcgatttcatcgctccggctatcacccataacccgctctctgatcatcaccagaaactgctgtctaacttcttcgcccagaccgaagcgctggcgtttggtaaatcccgcgaagtggttgagcaggaatatcgtgatcagggtaaagatccggcaacgcttgactacgtggtgccgttcaaagtattcgaaggtaaccgcccgaccaactccatcctgctgcgtgaaatcactccgttcagcctgggtgcgttgattgcgctgtatgagcacaaaatctttactcagggcgtgatcctgaacatcttcaccttcgaccagtggggcgtggaactgggtaaacagctggcgaaccgtattctgccagagctgaaagatgataaagaaatcagcagccacgatagctcgaccaatggtctgattaaccgctataaagcgtggcgcggttaa

SEQ ID NO: 2 - protein, 549 aa, Escherichia coli
MKNINPTQTAAWQALQKHFDEMKDVTIADLFAKDGDRFSKFSATFDDQMLVDYSKNRITEETLAKLQDLAKECDLAGAIKSMFSGEKINRTENRAVLHVALRNRSNTPILVDGKDVMPEVNAVLEKMKTFSEAIISGEWKGYTGKAITDVVNIGIGGSDLGPYMVTEALRPYKNHLNMHFVSNVDGTHIAEVLKKVNPETTLFLVASKTFTTQETMTNAHSARDWFLKAAGDEKHVAKHFAALSTNAKAVGEFGIDTANMFEFWDWVGGRYSLWSAIGLSIVLSIGFDNFVELLSGAHAMDKHFSTTPAEKNLPVLLALIGIWYNNFFGAETEAILPYDQYMHRFAAYFQQGNMESNGKYVDRNGNVVDYQTGPIIWGEPGTNGQHAFYQLIHQGTKMVPCDFIAPAITHNPLSDHHQKLLSNFFAQTEALAFGKSREVVEQEYRDQGKDPATLDYVVPFKVFEGNRPTNSILLREITPFSLGALIALYEHKIFTQGVILNIFTFDQWGVELGKQLANRILPELKDDKEISSHDSSTNGLINRYKAWRG

SEQ ID NO: 3 - DNA, 1602 bp, Saccharomyces cerevisiae
atgctagaagataatattgctccaatcacctccgttaaagtagttaccgacaagtgcacgtacaaggacaacgagctgctcaccaagtacagctacgaaaatgctgtagttacgaagacagctagtggccgcttcgatgtaacgcccactgttcaagactacgtgttcaaacttgacttgaaaaagccggaaaaactaggaattatgctcattgggttaggtggcaacaatggctccactttagtggcctcggtattggcgaataagcacaatgtggagtttcaaactaaggaaggcgttaagcaaccaaactacttcggctccatgactcaatgttctaccttgaaactgggtatcgatgcggaggggaatgacgtttatgctccttttaactctctgttgcccatggttagcccaaacgactttgtcgtctctggttgggacatcaataacgcagatctatacgaagctatgcagagaagtcaagttctcgaatatgatctgcaacaacgcttgaaggcgaagatgtccttggtgaagcctcttccttccatttactaccctgatttcattgcagctaatcaagatgagagagccaataactgcatcaatttggatgaaaaaggcaacgtaaccacgaggggtaagtggacccatctgcaacgcatcagacgcgatatccagaatttcaaagaagaaaacgcccttgataaagtaatcgttctttggactgcaaatactgagaggtacgtagaagtatctcctggtgttaatgacaccatggaaaacctcttgcagtctattaagaatgaccatgaagagattgctccttccacgatctttgcagcagcatctatcttggaaggtgtcccctatattaatggttcaccgcagaatacttttgttcccggcttggttcagctggctgagcatgagggtacattcattgcgggagacgatctcaagtcgggacaaaccaagttgaagtctgttctggcccagttcttagtggatgcaggtattaaaccggtctccattgcatcctataaccatttaggcaataatgacggttataacttatctgctccaaaacaatttaggtctaaggagatttccaaaagttctgtcatagatgacatcatcgcgtctaatgatatcttgtacaatgataaactgggtaaaaaagttgaccactgcattgtcatcaaatatatgaagcccgtcggggactcaaaagtggcaatggacgagtattacagtgagttgatgttaggtggccataaccggatttccattcacaatgtttgcgaagattctttactggctacgcccttgatcatcgatcttttagtcatgactgagttttgtacaagagtgtcctataagaaggtggacccagttaaagaagatgctggcaaattcgagaacttttatccagttttaaccttcttgagttactggttaaaagctccattaacaagaccaggatttcacccggtgaatggcttaaacaagcaaagaaccgccttagaaaattttttaagattgttgattggattgccttctcaaaacgaactaagattcgaagagagattgttgtaa

SEQ ID NO: 4 - protein, 533 aa, Saccharomyces cerevisiae
MLEDNIAPITSVKVVTDKCTYKDNELLTKYSYENAVVTKTASGRFDVTPTVQDYVFKLDLKKPEKLGIMLIGLGGNNGSTLVASVLANKHNVEFQTKEGVKQPNYFGSMTQCSTLKLGIDAEGNDVYAPFNSLLPMVSPNDFVVSGWDINNADLYEAMQRSQVLEYDLQQRLKAKMSLVKPLPSIYYPDFIAANQDERANNCINLDEKGNVTTRGKWTHLQRIRRDIQNFKEENALDKVIVLWTANTERYVEVSPGVNDTMENLLQSIKNDHEEIAPSTIFAAASILEGVPYINGSPQNTFVPGLVQLAEHEGTFIAGDDLKSGQTKLKSVLAQFLVDAGIKPVSIASYNHLGNNDGYNLSAPKQFRSKEISKSSVIDDIIASNDILYNDKLGKKVDHCIVIKYMKPVGDSKVAMDEYYSELMLGGHNRISIHNVCEDSLLATPLIIDLLVMTEFCTRVSYKKVDPVKEDAGKFENFYPVLTFLSYWLKAPLTRPGFHPVNGLNKQRTALENFLRLLIGLPSQNELRFEERLL

SEQ ID NO: 5 - DNA, 804 bp, Escherichia coli
atgcatccgatgctgaacatcgccgtgcgcgcagcgcgcaaggcgggtaatttaattgccaaaaactatgaaaccccggacgctgtagaagcgagccagaaaggcagtaacgatttcgtgaccaacgtagataaagctgccgaagcggtgattatcgacacgattcgtaaatcttacccacagcacaccatcatcaccgaagaaagcggtgaacttgaaggtactgatcaggatgttcaatgggttatcgatccactggatggcactaccaactttatcaaacgtctgccgcacttcgcggtatctatcgctgttcgtatcaaaggccgcaccgaagttgctgtggtatacgatcctatgcgtaacgaactgttcaccgccactcgcggtcagggcgcacagctgaacggctaccgactgcgcggcagcaccgctcgcgatctcgacggtactattctggcgaccggcttcccgttcaaagcaaaacagtacgccactacctacatcaacatcgtcggcaaactgttcaacgaatgtgcagacttccgtcgtaccggttctgcggcgctggatctggcttacgtcgctgcgggtcgtgttgacggtttctttgaaatcggtctgcgcccgtgggacttcgccgcaggcgagctgctggttcgtgaagcgggcggcatcgtcagcgacttcaccggtggtcataactacatgctgaccggtaacatcgttgctggtaacccgcgcgttgttaaagccatgctggcgaacatgcgtgacgagttaagcgacgctctgaagcgttaa

SEQ ID NO: 6 - protein, 267 aa, Escherichia coli
MHPMLNIAVRAARKAGNLIAKNYETPDAVEASQKGSNDFVTNVDKAAEAVIIDTIRKSYPQHTIITEESGELEGTDQDVQWVIDPLDGTTNFIKRLPHFAVSIAVRIKGRTEVAVVYDPMRNELFTATRGQGAQLNGYRLRGSTARDLDGTILATGFPFKAKQYATTYINIVGKLFNECADFRRTGSAALDLAYVAAGRVDGFFEIGLRPWDFAAGELLVREAGGIVSDFTGGHNYMLTGNIVAGNPRVVKAMLANMRDELSDALKR

SEQ ID NO: 7 - DNA, 1035 bp, Bacillus subtilis
atgagtttacgtattggcgtaattggaactggagcaatcggaaaagaacatattaaccgtatcacgaacaagctgtcaggcgcggaaattgtagctgtaacggatgttaatcaagaagctgcacaaaaggtcgttgagcaataccaattaaacgcgacggtttatccgaatgatgacagcttgcttgcagacgaaaatgtagacgctgttttagtgacaagctgggggcctgcgcatgagtcaagcgtgctgaaagcgattaaagcccagaaatatgtgttctgtgaaaaaccgctcgcgacaacggctgaaggatgcatgcgcattgtcgaagaagaaatcaaagtgggcaaacgccttgttcaagtcggcttcatgcgccgttatgacagcggttacgtacagctgaaagaagcgctcgataatcatgtcaacggcgagcctcttatgattcactgcgcgcaccgcaacccgactgtaggagataactatacaacggatatggctgtagtcgacacgcttgttcatgaaattgacgtgctccactggctcgtcaatgatgactacgagtccgttcaagtcatctatccgaaaaaatcaaaaaacgcgcttccacatttaaaagatccgcaaatcgtcgtgattgaaacaaaaggcggtatcgtcatcaatgctgaaatctatgtgaactgtaaatacggctatgacattcaatgtgaaatcgtcggagaagacggcatcatcaagcttcccgagccatcaagcatcagcttgagaaaagaaggcagattcagcactgatattttgatggattggcagagacgctttgtcgctgcgtatgatgtggaaatccaagactttattgattcgattcaaaagaaaggcgaggtcagcggaccgacggcatgggacggctatattgctgctgtcacgactgacgcgtgtgtaaaagcccaggaatctggacaaaaagaaaaggttgaattgaaggaaaaaccggaattctatcaatcttttacaacagttcaaaactaa

SEQ ID NO: 8 - protein, 344 aa, Bacillus subtilis
MSLRIGVIGTGAIGKEHINRITNKLSGAEIVAVTDVNQEAAQKVVEQYQLNATVYPNDDSLLADENVDAVLVTSWGPAHESSVLKAIKAQKYVFCEKPLATTAEGCMRIVEEEIKVGKRLVQVGFMRRYDSGYVQLKEALDNHVNGEPLMIHCAHRNPTVGDNYTTDMAVVDTLVHEIDVLHWLVNDDYESVQVIYPKKSKNALPHLKDPQIVVIETKGGIVINAEIYVNCKYGYDIQCEIVGEDGIIKLPEPSSISLRKEGRFSTDILMDWQRRFVAAYDVEIQDFIDSIQKKGEVSGPTAWDGYIAAVTTDACVKAQESGQKEKVELKEKPEFYQSFTTVQN

SEQ ID NO: 9 - DNA, 894 bp, Bacillus subtilis
atgggcaaaaatgaaatcctgtggggaatcgctcccattgggtggcggaatgatgacatgcctgaaattggagcgggaaatacacttcagcatttgttaagtgatatcgttgtcgcacgttttcaaggcacggaggtcgggggctttttccccgaacctgccatcctgaacaaagagctgaagcttcggaacttacgcattgcaggaaaatggttcagcagttttattttgcgtgacggacttggtgaagcggcaaagacatttaccctgcattgtgagtatttgcagcaagtaaacgcggatgtcgcagttgtctctgaacaaacgtacagcgtgcaaagcttggagaaaaatgtgttcacagagaagccgcactttacggatgatgaatgggagcggctttgcgaagggctgaatcaccttggcgaaattgccgctcagcatggcttgaagcttgtctatcatcatcatctcggcactggtgtccaaacagcggaagaagtggaccgcctgatggcaggaacagaccctgcgcatgtacacctcctctatgatacaggccatgcgtatatttctgacggcgattacatggggatgcttgagaagcatatcggccgcattaagcatgtgcactttaaggatgcccgcctgaatgtcatggaacaatgcaggctcgaaggacaatcgttccggcaatcatttttaaaaggcatgtttacggttcccggtgacggctgcattgactttagagaagtatatcagctgctgttgaagcacagttattccggatggattgtcattgaagctgaacaagaccccgatgttgcaaacccgctggagtatgcattgattgcgagaaactatattgatcagcagttgttggatctggcttaa

SEQ ID NO: 10 - protein, 297 aa, Bacillus subtilis
MGKNEILWGIAPIGWRNDDMPEIGAGNTLQHLLSDIVVARFQGTEVGGFFPEPAILNKELKLRNLRIAGKWFSSFILRDGLGEAAKTFTLHCEYLQQVNADVAVVSEQTYSVQSLEKNVFTEKPHFTDDEWERLCEGLNHLGEIAAQHGLKLVYHHHLGTGVQTAEEVDRLMAGTDPAHVHLLYDTGHAYISDGDYMGMLEKHIGRIKHVHFKDARLNVMEQCRLEGQSFRQSFLKGMFTVPGDGCIDFREVYQLLLKHSYSGWIVIEAEQDPDVANPLEYALIARNYIDQQLLDLA

SEQ ID NO: 11 - DNA, 1710 bp, Bacillus subtilis
atggcgcatgcggccatggcgtacagcaagcaaatgctgagaagaaaaatatatgcggtgtctacatccgtcggacctggagcggctaacttggtggcggcagccggcactgcattggcaaataatatcccagttctcttgattccggcagacacatttgcgacaagacagccagaccctgtactgcagcaaatggagcaagaatacagtgcggcgattacgacaaacgatgcattgaagcctgtatcgagatactgggaccgcattacgcgccctgagcagctgatgagcagtttgctgcgcgcgtttgaagtcatgactgatccggcaaaagcaggtccggcaacgatttgtatttctcaggatgttgaaggggaagcatacgattttgatgaaagtttcttcgtcaaacgggttcactatattgatcgcatgcagccgagcgagcgcgagcttcaaggtgcggcggagctgattaaatccagcaaaaaacctgtgattctcgtcggcggaggcgcaaaatattccggtgcgcgcgatgaattggttgccatttctgaagcctataacattccgttagttgaaacgcaggcaggaaagtctaccgttgaggcagattttgcgaacaaccttggcggaatgggcatcacaggtacacttgcggcaaacaaagcggcccgccaagctgatttgattatcggcatcggcacaaggtatacagattttgcgacatcctctaagaccgcttttgattttgataaagcgaagtttttgaacattaacgtcagccgaatgcaggcgtataaacttgatgcattccaagtggtggcggatgcgaaagtgacgcttggcaaactgcacggcttgcttgaaggctatgagagcgagttcggcacaacgatccgggagctgaaggacgaatggctagctgaacgcgagcgtctcagcaaagtgacgtttaagcgggaagcatttgatccggaaatcaaaaatcacttttctcaagaggtcctgaatgaatatgctgacgcgttgaatacggagcttccgcaaacaacggcattgctgacaatcaacgagacgattcctgaagacagcgtcatcatctgttcagcaggctcactcccaggagatttgcagcgtctgtggcattctaatgttccgaatacgtatcacctggagtatggatattcttgcatgggctatgaagtgtccgggacactcggtcttaaactggcccaccctgacagagaagtgtattcaatcgtcggagacggcagcttcctgatgcttcattctgaactgatcacggcgattcagtacaacaagaaaatcaatgtgctgctctttgacaactcaggattcggatgcatcaacaacctgcaaatggatcacggcagcggcagctactattgcgagttccgcacagatgacaaccaaatcctgaatgtcgattacgcgaaagtcgctgagggatacggcgcgaaaacctaccgtgcaaacacagtagaagaattaaaagctgcgttagaggatgcgaagaaacaggatgtatcaacattaattgaaatgaaggtgctgcctaaaacaatgacggacggctatgacagctggtggcatgtcggggtggcagaggtatctgaacaagaaagcgttcagaaagcatacgaagcaaaagagaaaaagctggaatctgcgaagcagtattag

SEQ ID NO: 12 - protein, 569 aa, Bacillus subtilis
MAHAAMAYSKQMLRRKIYAVSTSVGPGAANLVAAAGTALANNIPVLLIPADTFATRQPDPVLQQMEQEYSAAITTNDALKPVSRYWDRITRPEQLMSSLLRAFEVMTDPAKAGPATICISQDVEGEAYDFDESFFVKRVHYIDRMQPSERELQGAAELIKSSKKPVILVGGGAKYSGARDELVAISEAYNIPLVETQAGKSTVEADFANNLGGMGITGTLAANKAARQADLIIGIGTRYTDFATSSKTAFDFDKAKFLNINVSRMQAYKLDAFQVVADAKVTLGKLHGLLEGYESEFGTTIRELKDEWLAERERLSKVTFKREAFDPEIKNHFSQEVLNEYADALNTELPQTTALLTINETIPEDSVIICSAGSLPGDLQRLWHSNVPNTYHLEYGYSCMGYEVSGTLGLKLAHPDREVYSIVGDGSFLMLHSELITAIQYNKKINVLLFDNSGFGCINNLQMDHGSGSYYCEFRTDDNQILNVDYAKVAEGYGAKTYRANTVEELKAALEDAKKQDVSTLIEMKVLPKTMTDGYDSWWHVGVAEVSEQESVQKAYEAKEKKLESAKQY

SEQ ID NO: 13 - DNA, 816 bp, Bacillus subtilis
atgagttatttgttgcgtaagccgcagtcgcatgaagtgtctaatggggtcaaactcgtgcacgaagtaacgacatccaactctgatctcacttatgtagagtttaaagtgttagatcttgcatcaggttcaagctatacagaagaattgaaaaaacaagaaatctgtattgtggcggtaacggggaaaattacagtgacagatcatgagtcgacttttgagaatatcggcacgcgcgaaagctattttgaacgaaaaccgacagacagcgtctatatttcaaatgaccgtgcatttgagatcacagcggtcagcgacgcaagagtggcgctttgctattctccatcggaaaagcagcttccgacaaagctgatcaaagcggaagacaacggaattgagcatcgcgggcaattttcaaacaaacgtactgttcataacattcttccggattcagacccttcagctaacagtctattagtagttgaagtctatacagacagcggcaactggtccagctacccgcctcacaaacatgaccaagacaacttgccggaagaatctttcttagaagaaacgtactaccatgagttagacccgggacagggctttgtgtttcagcgcgtatacacagatgaccgttctattgacgagacaatgactgtgggaaatgaaaacgttgtcatcgttcctgcgggataccacccggtaggcgttccggacggatacacatcctactatttaaatgtcatggcagggccgacgcgaaaatggaagttttataacgacccggcgcatgaatggattttagaacgctaa

SEQ ID NO: 14 - protein, 271 aa, Bacillus subtilis
MSYLLRKPQSHEVSNGVKLVHEVTTSNSDLTYVEFKVLDLASGSSYTEELKKQEICIVAVTGKITVTDHESTFENIGTRESYFERKPTDSVYISNDRAFEITAVSDARVALCYSPSEKQLPTKLIKAEDNGIEHRGQFSNKRTVHNILPDSDPSANSLLVVEVYTDSGNWSSYPPHKHDQDNLPEESFLEETYYHELDPGQGFVFQRVYTDDRSIDETMTVGNENVVIVPAGYHPVGVPDGYTSYYLNVMAGPTRKWKFYNDPAHEWILER

SEQ ID NO: 15 - DNA, 1020 bp, Bacillus subtilis
atggattttagaacgctaacaagtgaggagtggctgtttacgatgaagtatacattcaatgaagagaaggcttttgatattgttgccatcggccgggcatgtattgatctgaacgcagtcgaatacaaccgcccaatggaagaaacgatgacattttcgaaatatgtcggcggttcacctgcgaatatcgcgatcggcagcgcgaagcttggcttaaaagcgggcttcatcggcaaaattccggatgaccagcatggaagattcatagagtcctatatgagaaagaccggcgtggatactacacagatgattgttgatcaagatggacacaaagcaggccttgcatttacagaaatcctcagccctgaagaatgcagcatcttaatgtatcgcgatgatgtggcggatctttatcttgagccttcagaggtaagtgaggactatatcgcaaatgcgaaaatgctgcttgtctccgggacagcgctcgccaaaagcccgtcacgggaagcggtgttaaaagctgttcaatacgcgaaaaagcatcaggttaaggtggtattcgaactggattaccggccatatacgtggcagtcatcagatgaaacagccgtttattattctttggttgccgagcagtctgatatcgtcatcggcacacgcgatgaatttgatgtgatggaaaaccgcacaggcggaagcaatgaagaatccgtcaatcatttatttggccattcagccgacctcgttgtcatcaaacacggcgtcgaaggctcttacgcatacagcaaatccggcgaggtattccgcgctcaagcgtacaagacaaaagtgctgaaaacctttggggccggtgactcctatgcgtcagcctttatctatggccttgtcagcggaaaagacattgaaacggcattgaaatacggcagtgcttcagcctccattgtggtgagcaagcacagttcgtcagaagcgatgccgactgcggaagaaatcgaacagcttattgaagcacagtcataa

SEQ ID NO: 16 - protein, 339 aa, Bacillus subtilis
MDFRTLTSEEWLFTMKYTFNEEKAFDIVAIGRACIDLNAVEYNRPMEETMTFSKYVGGSPANIAIGSAKLGLKAGFIGKIPDDQHGRFIESYMRKTGVDTTQMIVDQDGHKAGLAFTEILSPEECSILMYRDDVADLYLEPSEVSEDYIANAKMLLVSGTALAKSPSREAVLKAVQYAKKHQVKVVFELDYRPYTWQSSDETAVYYSLVAEQSDIVIGTRDEFDVMENRTGGSNEESVNHLFGHSADLVVIKHGVEGSYAYSKSGEVFRAQAYKTKVLKTFGAGDSYASAFIYGLVSGKDIETALKYGSASASIVVSKHSSSEAMPTAEEIEQLIEAQS

SEQ ID NO: 17 - DNA, 894 bp, Bacillus subtilis
atggataaaggaggggtgaatatggcttttgtatcgatgaaagagcttcttgaagatgcaaagcgggagcaatatgcaattggccagtttaatatcaacggcctgcaatggacgaaggcgattttgcaggcggcgcaaaaggagcaatcaccggtcatcgccgcggcttccgatcgcctggtcgactatttaggcggatttaaaacgattgccgccatggtcggcgcgttaatagaggacatggcgattaccgttccggtcgtgcttcatctcgatcacggcagcagtgcggaacgctgcagacaggccattgatgccggattcagctcagtgatgattgacggctcccatcagccgattgacgagaatatcgcgatgacaaaagaagtcaccgattatgccgcaaaacacggcgtgtcagtagaagccgaagtcggcacggtcggcggaatggaagacggactggtcggcggggtccgctatgcggatatcacggaatgtgagcggatcgttaaagaaaccaatatcgacgcgctggccgccgccctcggctctgtacacggcaaatatcagggtgagccgaatctcggatttaaggaaatggaggctatctcccgcatgactgatattcccctcgttcttcacggggcatccgggattccgcaggatcagatcaaaaaagccatcacgctcggccacgcgaagatcaatatcaatacggaatgtatggtagcgtggacagacgaaacacgccgcatgtttcaggaaaacagcgatctgtacgaaccgcgcggctatttgacacccggcattgaagccgtggaagagacagtgcgaagcaaaatgagagagttcggatcagccggtaaagcagctaagcagcaggtcggctaa

SEQ ID NO: 18 - protein, 297 aa, Bacillus subtilis
MDKGGVNMAFVSMKELLEDAKREQYAIGQFNINGLQWTKAILQAAQKEQSPVIAAASDRLVDYLGGFKTIAAMVGALIEDMAITVPVVLHLDHGSSAERCRQAIDAGFSSVMIDGSHQPIDENIAMTKEVTDYAAKHGVSVEAEVGTVGGMEDGLVGGVRYADITECERIVKETNIDALAAALGSVHGKYQGEPNLGFKEMEAISRMTDIPLVLHGASGIPQDQIKKAITLGHAKININTECMVAWTDETRRMFQENSDLYEPRGYLTPGIEAVEETVRSKMREFGSAGKAAKQQVG

SEQ ID NO: 19 - DNA, 1440 bp, Escherichia coli
atgtcagtacccgttcaacatcctatgtatatcgatggacagtttgttacctggcgtggagacgcatggattgatgtggtaaaccctgctacagaggctgtcatttcccgcatacccgatggtcaggccgaggatgcccgtaaggcaatcgatgcagcagaacgtgcacaaccagaatgggaagcgttgcctgctattgaacgcgccagttggttgcgcaaaatctccgccgggatccgcgaacgcgccagtgaaatcagtgcgctgattgttgaagaagggggcaagatccagcagctggctgaagtcgaagtggcttttactgccgactatatcgattacatggcggagtgggcacggcgttacgagggcgagattattcaaagcgatcgtccaggagaaaatattcttttgtttaaacgtgcgcttggtgtgactaccggcattctgccgtggaacttcccgttcttcctcattgcccgcaaaatggctcccgctcttttgaccggtaataccatcgtcattaaacctagtgaatttacgccaaacaatgcgattgcattcgccaaaatcgtcgatgaaataggccttccgcgcggcgtgtttaaccttgtactggggcgtggtgaaaccgttgggcaagaactggcgggtaacccaaaggtcgcaatggtcagtatgacaggcagcgtctctgcaggtgagaagatcatggcgactgcggcgaaaaacatcaccaaagtgtgtctggaattggggggtaaagcaccagctatcgtaatggacgatgccgatcttgaactggcagtcaaagccatcgttgattcacgcgtcattaatagtgggcaagtgtgtaactgtgcagaacgtgtttatgtacagaaaggcatttatgatcagttcgtcaatcggctgggtgaagcgatgcaggcggttcaatttggtaaccccgctgaacgcaacgacattgcgatggggccgttgattaacgccgcggcgctggaaagggtcgagcaaaaagtggcgcgcgcagtagaagaaggggcgagagtggcgttcggtggcaaagcggtagaggggaaaggatattattatccgccgacattgctgctggatgttcgccaggaaatgtcgattatgcatgaggaaacctttggcccggtgctgccagttgtcgcatttgacacgctggaagatgctatctcaatggctaatgacagtgattacggcctgacctcatcaatctatacccaaaatctgaacgtcgcgatgaaagccattaaagggctgaagtttggtgaaacttacatcaaccgtgaaaacttcgaagctatgcaaggcttccacgccggatggcgtaaatccggtattggcggcgcagatggtaaacatggcttgcatgaatatctgcagacccaggtggtttatttacagtcttaa

SEQ ID NO: 20 - protein, 479 aa, Escherichia coli
MSVPVQHPMYIDGQFVTWRGDAWIDVVNPATEAVISRIPDGQAEDARKAIDAAERAQPEWEALPAIERASWLRKISAGIRERASEISALIVEEGGKIQQLAEVEVAFTADYIDYMAEWARRYEGEIIQSDRPGENILLFKRALGVTTGILPWNFPFFLIARKMAPALLTGNTIVIKPSEFTPNNAIAFAKIVDEIGLPRGVFNLVLGRGETVGQELAGNPKVAMVSMTGSVSAGEKIMATAAKNITKVCLELGGKAPAIVMDDADLELAVKAIVDSRVINSGQVCNCAERVYVQKGIYDQFVNRLGEAMQAVQFGNPAERNDIAMGPLINAAALERVEQKVARAVEEGARVAFGGKAVEGKGYYYPPTLLLDVRQEMSIMHEETFGPVLPVVAFDTLEDAISMANDSDYGLTSSIYTQNLNVAMKAIKGLKFGETYINRENFEAMQGFHAGWRKSGIGGADGKHGLHEYLQTQVVYLQS

SEQ ID NO: 21 - DNA, 1515 bp, Rhizobium leguminosarum
gtgagcaaccatcttttcgacgccatgcgggccgccgcgcccggtaacgcaccattcatccggatcgataacacgcgcacatggacctatgacgacgccttcgctctttccggccgcattgccagcgcgatggacgcgctcggcattcgccccggcgaccgcgttgcggtgcaggtcgagaaaagtgccgaggcattgatcctctatctcgcctgtcttcgaagcggcgccgtctacctgccgctcaacaccgcctatacgctggctgagctcgattattttatcggcgatgcggagccgcgtttggtggttgtcgcatcgtcggctcgagcgggcgtggagacaatcgccaagccccgcggtgcgatcgtcgaaactctcgacgctgctggcagcggctcgttgctggatctcgcccgcgacgagccggccgactttgtcgatgcctcgcgctccgccgatgatctggcggcgatcctctacacgtccggaacgacgggacgctccaagggggcgatgctcacgcatgggaacctgctctcgaacgccctgaccttgcgagatttttggcgcgtcaccgccggcgatcgactgatccatgccttgccgatcttccacacgcatggactgttcgtcgccacgaacgtcacactgctcgccggcgcctcgatgttcctgctgtcgaagttcgacccggaggagatcctgtcgctgatgccgcaggcaacgatgctgatgggcgtgccgaccttctacgtgcgcctcctgcagagcccgcgcctcgacaagcaagcggtcgccaacatccgcctcttcatttccggttcggctccactgcttgcagaaacacataccgagttccaggcacgtaccggtcacgccattctcgagcgctacggcatgacggaaaccaatatgaacacgtccaacccttatgaggggaaacggattgccggaacggtcggcttcccgctgcctgatgtgacggtgcgcgtcaccgatcccgccaccgggctcgcgctgccgcccgaacaaaccggcatgatcgagatcaaggggccgaacgttttcaagggctattggcgcatgcccgaaaaaaccgcggccgaattcaccgccgacggtttcttcatcagcggcgatctcggcaagatcgaccgcgacggttatgtccacatcgtcggccgcggcaaggatctggtgatttcgggtggatacaacatctatccgaaagaggttgagggcgagatcgaccagatcgagggtgtggttgagagcgctgtgatcggcgtgccgcatcccgatttcggagaaggcgtaacggccgtcgtcgtgcgcaagcccggcgctgccctcgatgaaaaggccatcgtcagcgccctccaggaccggctcgcgcgctacaaacaacccaagcgcatcatctttgcagaggacttgccgcgcaacacgatgggtaaggttcagaaaaacatcctgcggcagcaatacgccgatctttataccaggacgtaa

SEQ ID NO: 22 - protein, 504 aa, Rhizobium leguminosarum
MSNHLFDAMRAAAPGNAPFIRIDNTRTWTYDDAFALSGRIASAMDALGIRPGDRVAVQVEKSAEALILYLACLRSGAVYLPLNTAYTLAELDYFIGDAEPRLVVVASSARAGVETIAKPRGAIVETLDAAGSGSLLDLARDEPADFVDASRSADDLAAILYTSGTTGRSKGAMLTHGNLLSNALTLRDFWRVTAGDRLIHALPIFHTHGLFVATNVTLLAGASMFLLSKFDPEEILSLMPQATMLMGVPTFYVRLLQSPRLDKQAVANIRLFISGSAPLLAETHTEFQARTGHAILERYGMTETNMNTSNPYEGKRIAGTVGFPLPDVTVRVTDPATGLALPPEQTGMIEIKGPNVFKGYWRMPEKTAAEFTADGFFISGDLGKIDRDGYVHIVGRGKDLVISGGYNIYPKEVEGEIDQIEGVVESAVIGVPHPDFGEGVTAVVVRKPGAALDEKAIVSALQDRLARYKQPKRIIFAEDLPRNTMGKVQKNILRQQYADLYTRT

SEQ ID NO: 23 - DNA, 3898 bp, Chloroflexus aurantiacus
aacatcatggtatactatacctatcgataattcttcaactaattgcataacagaacagcgatggcgacgggggagtccatgagcggaacaggacgactggcaggaaagattgcgttaattaccggtggcgccggcaatatcggcagtgaattgacacgtcgctttctcgcagagggagcgacggtcattattagtggacggaatcgggcgaagttgaccgcactggccgaacggatgcaggcagaggcaggagtgccggcaaagcgcatcgatctcgaagtcatggatgggagtgatccggtcgcggtacgtgccggtatcgaagcgattgtggcccgtcacggccagatcgacattctggtcaacaatgcaggaagtgccggtgcccagcgtcgtctggccgagattccactcactgaagctgaattaggccctggcgccgaagagacgcttcatgccagcatcgccaatttacttggtatgggatggcatctgatgcgtattgcggcacctcatatgccggtaggaagtgcggtcatcaatgtctcgaccatcttttcacgggctgagtactacgggcggattccgtatgtcacccctaaagctgctcttaatgctctatctcaacttgctgcgcgtgagttaggtgcacgtggcatccgcgttaatacgatctttcccggcccgattgaaagtgatcgcatccgtacagtgttccagcgtatggatcagctcaaggggcggcccgaaggcgacacagcgcaccattttttgaacaccatgcgattgtgtcgtgccaacgaccagggcgcgcttgaacgtcggttcccctccgtcggtgatgtggcagacgccgctgtctttctggccagtgccgaatccgccgctctctccggtgagacgattgaggttacgcacggaatggagttgccggcctgcagtgagaccagcctgctggcccgtactgatctgcgcacgattgatgccagtggccgcacgacgctcatctgcgccggcgaccagattgaagaggtgatggcgctcaccggtatgttgcgtacctgtgggagtgaagtgatcatcggcttccgttcggctgcggcgctggcccagttcgagcaggcagtcaatgagagtcggcggctggccggcgcagactttacgcctcccattgccttgccactcgatccacgcgatccggcaacaattgacgctgtcttcgattggggggccggcgagaataccggcgggattcatgcagcggtgattctgcctgctaccagtcacgaaccggcaccgtgcgtgattgaggttgatgatgagcgggtgctgaattttctggccgatgaaatcaccgggacaattgtgattgccagtcgcctggcccgttactggcagtcgcaacggcttacccccggcgcacgtgcgcgtgggccgcgtgtcatttttctctcgaacggtgccgatcaaaatgggaatgtttacggacgcattcaaagtgccgctatcggtcagctcattcgtgtgtggcgtcacgaggctgaacttgactatcagcgtgccagcgccgccggtgatcatgtgctgccgccggtatgggccaatcagattgtgcgcttcgctaaccgcagccttgaagggttagaatttgcctgtgcctggacagctcaattgctccatagtcaacgccatatcaatgagattaccctcaacatccctgccaacattagcgccaccaccggcgcacgcagtgcatcggtcggatgggcggaaagcctgatcgggttgcatttggggaaagttgccttgattaccggtggcagcgccggtattggtgggcagatcgggcgcctcctggctttgagtggcgcgcgcgtgatgctggcagcccgtgatcggcataagctcgaacagatgcaggcgatgatccaatctgagctggctgaggtggggtataccgatgtcgaagatcgcgtccacattgcaccgggctgcgatgtgagtagcgaagcgcagcttgcggatcttgttgaacgtaccctgtcagcttttggcaccgtcgattatctgatcaacaacgccgggatcgccggtgtcgaagagatggttatcgatatgccagttgagggatggcgccataccctcttcgccaatctgatcagcaactactcgttgatgcgcaaactggcgccgttgatgaaaaaacagggtagcggttacatccttaacgtctcatcatactttggcggtgaaaaagatgcggccattccctaccccaaccgtgccgattacgccgtctcgaaggctggtcagcgggcaatggccgaagtctttgcgcgcttccttggcccggagatacagatcaatgccattgcgccgggtccggtcgaaggtgatcgcttgcgcggtaccggtgaacgtcccggcctctttgcccgtcgggcgcggctgattttggagaacaagcggctgaatgagcttcacgctgctcttatcgcggctgcgcgcaccgatgagcgatctatgcacgaactggttgaactgctcttacccaatgatgtggccgcactagagcagaatcccgcagcacctaccgcgttgcgtgaactggcacgacgttttcgcagcgaaggcgatccggcggcatcatcaagcagtgcgctgctgaaccgttcaattgccgctaaattgctggctcgtttgcataatggtggctatgtgttgcctgccgacatctttgcaaacctgccaaacccgcccgatcccttcttcacccgagcccagattgatcgcgaggctcgcaaggttcgtgacggcatcatggggatgctctacctgcaacggatgccgactgagtttgatgtcgcaatggccaccgtctattaccttgccgaccgcaatgtcagtggtgagacattccacccatcaggtggtttgcgttacgaacgcacccctaccggtggcgaactcttcggcttgccctcaccggaacggctggcggagctggtcggaagcacggtctatctgataggtgaacatctgactgaacaccttaacctgcttgcccgtgcgtacctcgaacgttacggggcacgtcaggtagtgatgattgttgagacagaaaccggggcagagacaatgcgtcgcttgctccacgatcacgtcgaggctggtcggctgatgactattgtggccggtgatcagatcgaagccgctatcgaccaggctatcactcgctacggtcgcccagggccggtcgtctgtacccccttccggccactgccgacggtaccactggtcgggcgtaaagacagtgactggagcacagtgttgagtgaggctgaatttgccgagttgtgcgaacaccagctcacccaccatttccgggtagcgcgcaagattgccctgagtgatggtgccagtctcgcgctggtcactcccgaaactacggctacctcaactaccgagcaatttgctctggctaacttcatcaaaacgacccttcacgcttttacggctacgattggtgtcgagagcgaaagaactgctcagcgcattctgatcaatcaagtcgatctgacccggcgtgcgcgtgccgaagagccgcgtgatccgcacgagcgtcaacaagaactggaacgttttatcgaggcagtcttgctggtcactgcaccactcccgcctgaagccgatacccgttacgccgggcggattcatcgcggacgggcgattaccgtgtaaattctacgccacaggaaccactaccaaaccagcatagtaagagaacgatagagacgttgcaatgcgacgtctctatcatatttccggccccccctagacaaacccccacgtcttcgtgtagactagaaacaggaggctgtatgcacgtccaacaaga

SEQ ID NO: 24 - protein, 1220 aa, Chloroflexus aurantiacus
MSGTGRLAGKIALITGGAGNIGSELTRRFLAEGATVIISGRNRAKLTALAERMQAEAGVPAKRIDLEVMDGSDPVAVRAGIEAIVARHGQIDILVNNAGSAGAQRRLAEIPLTEAELGPGAEETLHASIANLLGMGWHLMRIAAPHMPVGSAVINVSTIFSRAEYYGRIPYVTPKAALNALSQLAARELGARGIRVNTIFPGPIESDRIRTVFQRMDQLKGRPEGDTAHHFLNTMRLCRANDQGALERRFPSVGDVADAAVFLASAESAALSGETIEVTHGMELPACSETSLLARTDLRTIDASGRTTLICAGDQIEEVMALTGMLRTCGSEVIIGFRSAAALAQFEQAVNESRRLAGADFTPPIALPLDPRDPATIDAVFDWGAGENTGGIHAAVILPATSHEPAPCVIEVDDERVLNFLADEITGTIVIASRLARYWQSQRLTPGARARGPRVIFLSNGADQNGNVYGRIQSAAIGQLIRVWRHEAELDYQRASAAGDHVLPPVWANQIVRFANRSLEGLEFACAWTAQLLHSQRHINEITLNIPANISATTGARSASVGWAESLIGLHLGKVALITGGSAGIGGQIGRLLALSGARVMLAARDRHKLEQMQAMIQSELAEVGYTDVEDRVHIAPGCDVSSEAQLADLVERTLSAFGTVDYLINNAGIAGVEEMVIDMPVEGWRHTLFANLISNYSLMRKLAPLMKKQGSGYILNVSSYFGGEKDAAIPYPNRADYAVSKAGQRAMAEVFARFLGPEIQINAIAPGPVEGDRLRGTGERPGLFARRARLILENKRLNELHAALIAAARTDERSMHELVELLLPNDVAALEQNPAAPTALRELARRFRSEGDPAASSSSALLNRSIAAKLLARLHNGGYVLPADIFANLPNPPDPFFTRAQIDREARKVRDGIMGMLYLQRMPTEFDVAMATVYYLADRNVSGETFHPSGGLRYERTPTGGELFGLPSPERLAELVGSTVYLIGEHLTEHLNLLARAYLERYGARQVVMIVETETGAETMRRLLHDHVEAGRLMTIVAGDQIEAAIDQAITRYGRPGPVVCTPFRPLPTVPLVGRKDSDWSTVLSEAEFAELCEHQLTHHFRVARKIALSDGASLALVTPETTATSTTEQFALANFIKTTLHAFTATIGVESERTAQRILINQVDLTRRARAEEPRDPHERQQELERFIEAVLLVTAPLPPEADTRYAGRIHRGRAITV

SEQ ID NO: 25 - DNA, 747 bp, Escherichia coli
atgatcgttttagtaactggagcaacggcaggttttggtgaatgcattactcgtcgttttattcaacaagggcataaagttatcgccactggccgtcgccaggaacggttgcaggagttaaaagacgaactgggagataatctgtatatcgcccaactggacgttcgcaaccgcgccgctattgaagagatgctggcatcgcttcctgccgagtggtgcaatattgatatcctggtaaataatgccggcctggcgttgggcatggagcctgcgcataaagccagcgttgaagactgggaaacgatgattgataccaacaacaaaggcctggtatatatgacgcgcgccgtcttaccgggtatggttgaacgtaatcatggtcatattattaacattggctcaacggcaggtagctggccgtatgccggtggtaacgtttacggtgcgacgaaagcgtttgttcgtcagtttagcctgaatctgcgtacggatctgcatggtacggcggtgcgcgtcaccgacatcgaaccgggtctggtgggtggtaccgagttttccaatgtccgctttaaaggcgatgacggtaaagcagaaaaaacctatcaaaataccgttgcattgacgccagaagatgtcagcgaagccgtctggtgggtgtcaacgctgcctgctcacgtcaatatcaataccctggaaatgatgccggttacccaaagctatgccggactgaatgtccaccgtcagtaa

SEQ ID NO: 26 - protein, 248 aa, Escherichia coli
MIVLVTGATAGFGECITRRFIQQGHKVIATGRRQERLQELKDELGDNLYIAQLDVRNRAAIEEMLASLPAEWCNIDILVNNAGLALGMEPAHKASVEDWETMIDTNNKGLVYMTRAVLPGMVERNHGHIINIGSTAGSWPYAGGNVYGATKAFVRQFSLNLRTDLHGTAVRVTDIEPGLVGGTEFSNVRFKGDDGKAEKTYQNTVALTPEDVSEAVWWVSTLPAHVNINTLEMMPVTQSYAGLNVHRQ

(19) United States
(12) Patent Application Publication
(10) Pub. No.: US 2011/0125118 A1
(43) Pub. Date: May 26, 2011

(54) Production of an Organic Acid and/or Related Chemicals

(75) Inventor: Michael D. Lynch, Boulder, CO (US)

(73) Assignee: OPX Biotechnologies, Inc., Boulder, CO (US)

(21) Appl. No.: 12/952,149

(22) Filed: Nov. 22, 2010

Related U.S. Application Data
(60) Provisional application No. 61/292,092, filed on Jan. 4, 2010.
(60) Provisional application No. 61/291,740, filed on Dec. 31, 2009.
(60) Provisional application No. 61/263,249, filed on Nov. 20, 2009.

(51) Int. Cl. A61L 15/22; C12P 7/42; C07C 51/377; C12P 7/40; C12N 1/00; C12N 1/21; C12N 15/74; C07C 59/01; D04H 11/00

(52) U.S. Cl. 604/367; 435/146; 562/599; 435/136; 435/243; 435/252.3; 435/471; 562/579; 428/85

(57) ABSTRACT

The present invention relates to methods, systems and compositions, including genetically modified microorganisms, e.g., recombinant microorganisms, adapted to utilize one or more synthesis gas components in a microbial bio-production of one or more desired biomolecules and products of commercial interest.

### RELATED APPLICATIONS

This application claims priority from U.S. Provisional Application 61/263,249, filed Nov. 20, 2009, U.S. Provisional Application 61/291,740, filed Dec. 31, 2009, U.S. Provisional Application 61/292,092, filed Jan. 4, 2010. The entire contents of each application are hereby incorporated by reference in their entirety.

### STATEMENT REGARDING FEDERALLY SPONSORED DEVELOPMENT

This invention was made with partial United States Government support under DE-AR0000088 awarded by the United States Department of Energy. The United States Government may have certain rights in this invention.

### REFERENCE TO A SEQUENCE LISTING

The instant application contains a Sequence Listing which has been submitted in ASCII format via EFS-Web and is hereby incorporated by reference in its entirety. Said ASCII copy, created on Nov. 22, 2010, is named 112210Syngas-3HP_ST25.txt and is 74.6 kB in size.

### FIELD OF THE INVENTION

The present invention relates to methods, systems and compositions, including genetically modified microorganisms, e.g., recombinant microorganisms, adapted to utilize one or more synthesis gas components in a microbial bio-production of one or more desired biomolecules and products of commercial interest.

### BACKGROUND

Economic, environmental and political impacts of and longer-term concerns with the current petroleum-based economy have driven the development and commercialization of processes that convert renewable feed stocks to both fuels and chemicals that can replace those derived from petroleum feed stocks. Two important goals of these developing processes include cost competitiveness with petroleum processes and reduced or net zero carbon dioxide or green house gas emissions. One approach to achieving these goals is the development of biorefining processes that utilize microorganisms to convert renewable feedstock sources such as cellulosic biomass or waste mass into products that are traditionally derived from petroleum or that can replace petroleum derived products. The list of petroleum-derived products of commercial value is exhaustive but includes molecules that fit into both the fuels and the chemicals markets, the latter including various industrial chemicals.

Due to recent competition between biorefining and food consumption for grains such as corn, and for sugar, it is clear that the path to sustainable non-petroleum-based fuel and chemical bio-production will require use of a broad range of alternative renewable feedstocks. One approach that may employ a wide range of alternative renewable feedstocks involves the thermo-conversion under oxygen-limited conditions of various carbonaceous feedstocks into synthesis gas.

Synthesis gas, which is also known as “syngas,” as used herein is a mixture of gases comprising carbon monoxide (CO), carbon dioxide (CO₂), and hydrogen (H₂) (collectively or individually, “syngas components”). Generally, syngas may be produced from any biomass material by gasification, steam reforming, partial oxidation, and similar processes that introduce oxygen at less than the stoichiometric ratio for combustion of the biomass. In some processes, part of the biomass is combusted, releasing CO₂ and heat which drives syngas formation from the biomass. Biomass such as lignocellulosic feedstocks, agricultural wastes, forest products, and grasses may be converted to syngas. In general, any carbonaceous feedstock can be utilized, including coal, petroleum, and natural gas, but renewable carbonaceous feedstocks such as biomass are considered particularly suitable. Gas mixtures derived from hydrogen and carbon dioxide produced from routes other than gasification could also be considered equivalents to syngas. For example, carbon dioxide waste streams may be mixed with hydrogen produced via any source for example electrolysis, steam methane reforming or any other.

Syngas is a platform intermediate in the chemical and biorefining industries and has a vast number of uses. Syngas can be converted into alkanes, olefins, oxygenates, and alcohols. These chemicals can be blended into, or used directly as, diesel fuel, gasoline, and other liquid fuels. Processes have been developed to convert syngas into chemicals such as methanol and acetic acid, and into liquid fuels using Fischer-Tropsch chemistry.

Components of syngas may be utilized in various ways, including as feedstock for biorefining processes. Production of syngas can be desirable within the context of bioconversion using microorganisms, because renewable biomass or waste feedstocks—which can be difficult to directly convert using microorganism—can first be converted into basic electron-rich reductant molecules H₂ and CO which can be consumed by suitable microorganisms.

A review of biological conversions of syngas is provided by Robert C. Brown in Chapter 11, pp. 227-252, of “Biorefinery Systems—An Overview,” in Biorefineries—Industrial Processes and Products, B. Kamm et al., Wiley-VCH (2006). This chapter is incorporated by reference herein for this background and descriptions of basic gasification reactions and certain metabolic pathways. According to this reference, anaerobic microorganisms have been favored for utilization of syngas conversions; this is stated to be because anaerobic microorganisms employ very energy-efficient metabolic pathways.

For example, U.S. Pat. No. 6,340,581, issued Jan. 22, 2002 to James L. Gaddy, discloses a method and apparatus for converting waste gases in a bioreactor to various products including organic acids and alcohols. Anaerobic bacteria are utilized in the bioreactor. Numerous specific microorganism isolates are disclosed, such as in the Background section of US Patent Publication No. 2008/0057554, published Mar. 6, 2008 to R. L. Huhnke et al., and are stated to be used for production of biofuels and/or chemicals from syngas components (collectively, biomolecules of interest). An emphasis is placed on anaerobic microorganisms, particularly acetogens.

As to composition of the syngas components supplied to the microorganisms, the well-known water-gas shift reaction can be used to enrich for either the CO or the H2 component of syngas. The water-gas shift reaction converts CO and H₂O into H₂ and CO₂. The reverse reaction also occurs, and the equilibrium of the water-gas shift reaction will generally govern the species distribution unless kinetic limitations are present. The water-gas shift can be performed on clean (i.e., purified) syngas, raw syngas directly from a gasification or partial-oxidation process, or any other source of syngas.

There is a clear need for alternative routes to create both fuels and products currently derived from petroleum. Fossil fuels account for 95% of the world energy usage and consumption of these fossil fuels has increased significantly over the last several decades. Consistent with this increase, carbon dioxide emissions have also been on a steady rise. These emissions are the primary reason for global climate change [http://cnpublications.net/2009/04/24/biofuels-instead-of-gasoline/, Daniel Gorelick and guest blogger Chaitan Khosla and Harmit Vora, http://www.springerlink.com/content/t78151r4811p6n74/, Jaime Klapp, Jorge L Cervantes-Cota, Luis C Longoria-Gandara and Ruslan Gabbasov]. In addition to the environmental dilemma surrounding fossil fuels, there is also a federal interest in localizing energy production within the United States to reduce dependence on oil-producing foreign nations. Equally important, the localization of national energy production will lead to a growing American economy, thus creating more jobs. Microbial systems offer the potential for the biological production of numerous types of chemicals, including 3-hydroxyproprionic acid (3-HP, CAS No. 503-66-2). The organic acid 3-HP, in turn, may be converted, enzymatically or chemically, into a number of other products [Top Value Added Chemicals from Biomass, Volume 1—Results of Screening for Potential Candidates from Sugars and Synthesis Gas, T. Werpy and G. Petersen, Ed., PNNL, NREL, EERD, Office of Biomass Program (August 2004).].

By using syngas components to make 3-HP, 3-HP and its downstream products, such as those listed above, can be produced from domestic renewable resources, such as switchgrass, rapeseed, or waste oils. It would be particularly beneficial for microorganisms to consume syngas components to produce 3-HP to capture and contain the chemical energy released in the process. Among other benefits, lower-cost feedstocks can ultimately be utilized, thereby enhancing overall economics and flexibility.

Notwithstanding the above-noted and other advances in the field, there remains a need to provide specific and, in some cases, coordinated improvements in microorganisms and biorefinery systems in which they would be utilized in order to achieve robust and cost-effective bio-production of 3-HP and related biomolecules and products of interest from syngas components.

### SUMMARY

Some aspects of the invention relate to integrated thermochemical-biological processing facilities, in particular those that utilize genetically modified microorganisms. Other aspects relate to the methods utilized to construct such genetically modified microorganisms and their methods of use in the systems and facilities, including those focused on the use of syngas components to provide carbon and energy to genetically modified microorganisms. Other aspects teach the use of metabolic pathways described herein with one or more sugars as a carbon and energy source. Other aspects relate to methods of making 3-HP using any of the genetically modified microorganisms made according to the invention, and further making downstream products from the 3-HP so produced, either via enzymatic, chemical, thermal, or thermochemical processes, or any combination thereof.

In some embodiments, the invention relates to a method of making and/or using a genetically modified microorganism comprising providing to a selected microorganism at least one genetic modification to introduce or increase one or more enzymatic activities selected from the group consisting of phosphoglucose isomerase, inositol-1-phosphate synthase, inositol monophosphatase, myo-inositol dehydrogenase, myo-inosose-2-dehydratase, inositol 2-dehydrogenase, deoxy-D-gluconate isomerase, 5-dehydro-2-deoxygluconokinase, deoxyphophogluconate aldolase, and 3-hydroxy acid dehydrogenase. In some embodiments, the invention relates to a method of making and/or using a genetically modified microorganism comprising providing to a selected microorganism at least one genetic modification to introduce or increase one or more enzymatic activities selected from the group consisting of phosphoglucose isomerase, inositol-1-phosphate synthase, inositol monophosphatase, myo-inositol dehydrogenase, myo-inosose-2-dehydratase, inositol 2-dehydrogenase, deoxy-D-gluconate isomerase, 5-dehydro-2-deoxygluconokinase, deoxyphophogluconate aldolase, an aldehyde dehydrogenase, a malonyl-CoA synthase, and malonyl-CoA reductase. In some embodiments an introduced malonyl-CoA reductase may be bi-functional, and in other embodiments it may be mono-functional wherein the microorganism comprises, such as through providing, a 3-hydroxy acid dehydrogenase function. In various embodiments there may be two or more, three or more, four or more, five or more, six or more, seven or more, eight or more, nine or more, ten or more, and the like, up to all of the noted enzymatic activities, that are provided by the noted at least one genetic modification.

Further, in specific embodiments the genetic modifications, such as those used in the methods of the invention and in microorganism compositions of the invention, comprise adding one or more of the particular nucleic acid sequences provided in Table 1, incorporated herein, conservatively modified variants thereof, and/or functional variants thereof, so as to provide one or more desired enzymatic activity described in Table 1 and depicted as the numbered reactions in FIG. 1, also incorporated into this section. In various embodiments a functional variant may be obtained that demonstrates an indicated activity that is at least 10, 20, 30, 40, 50, 60 70, 80, 90, 100, or greater than 150 or 200 percent greater than the activity of the native, or starting, enzyme.

Also, the invention comprises a method of making a genetically modified microorganism comprising providing to a selected microorganism at least one genetic modification to introduce or increase one or more enzymatic activities provided by amino acid sequences having at least 50%, 60%, 70%, 80%, 85%, 90%, 92%, 95%, 96%, 97%, 98% or 99% sequence identity to one or more amino acid sequences selected from the group consisting of SEQ ID NO:002, SEQ ID NO:004, SEQ ID NO:006, SEQ ID NO:008, SEQ ID NO:010, SEQ ID NO:012, SEQ ID NO:014, SEQ ID NO:016, SEQ ID NO:018, SEQ ID NO:020, SEQ ID NO:022, SEQ ID NO:024, SEQ ID NO:026, and conservatively modified variants thereof.

Also, the invention comprises a method of making a genetically modified microorganism comprising providing to a selected microorganism at least one genetic modification comprising providing a polynucleotide comprising a nucleic acid sequence having at least 50%, 60%, 70%, 80%, 85%, 90%, 92%, 95%, 96%, 97%, 98% or 99% sequence identity to one or more nucleic acid sequences from the group consisting of SEQ ID NO:001, SEQ ID NO:003, SEQ ID NO:005, SEQ ID NO:007, SEQ ID NO:009, SEQ ID NO:011, SEQ ID NO:013, SEQ ID NO:015, SEQ ID NO:017, SEQ ID NO:019, SEQ ID NO:021, SEQ ID NO:023, SEQ ID NO:025, and conservatively modified variants thereof.

Also, for each of the respective nucleic acid and amino acid sequences provided herein, the invention comprises:

a. Any of the methods and compositions provided herein, having an amino acid sequence having at least 50%, 60%, 70%, 80%, 85%, 90%, 92%, 95%, 96%, 97%, 98% or 99% sequence identity to an amino acid sequence provided herein.

b. Any of the methods and compositions provided herein, having an amino acid sequence that is a functional variant of an amino acid sequence provided herein.

c. Any of the methods and compositions provided herein, having an amino acid sequence variant that stringently hybridizes to an amino acid sequence provided herein.

d. Any of the methods and compositions provided herein, having a polynucleotide (nucleic acid sequence) that encodes an amino acid sequence having at least 50%, 60%, 70%, 80%, 85%, 90%, 92%, 95%, 96%, 97%, 98% or 99% sequence identity to an amino acid sequence provided herein.

e. Any of the methods and compositions provided herein, having a polynucleotide (nucleic acid sequence) has at least 50%, 60%, 70%, 80%, 85%, 90%, 92%, 95%, 96%, 97%, 98% or 99% sequence identity to a polynucleotide sequence provided herein.

By amino acid and polynucleotide sequence (nucleic acid sequence) provided herein is meant one of the sequences of SEQ ID NO:001 to 035xx and the sequences of the enzymes shown in FIG. 1, discussed further herein.

The scope of the invention includes microorganisms made by the methods described herein, and culture systems employing these microorganisms to produce 3-HP which may then be converted enzymatically, catalytically (chemical conversion), and/or with thermal treatment to, for example, any of the following chemicals: polymerized-3-HP (poly-3-HP), acrylic acid (CAS No. 79-10-7), polyacrylic acid, methyl acrylate, acrylamide (CAS No. 79-06-01), acrylonitrile, propiolactone, ethyl 3-HP, malonic acid, and 1,3-propanediol. Any of these, which are referred to as downstream compounds, may therefore become a final product of interest in a method of the present invention. In various embodiments, the downstream compound of 3-HP is selected from the group consisting of acrylic acid, acrylamide, acrylonitrile, propiolactone, ethyl 3-HP, malonic acid, 1,3-propanediol, methyl acrylate, ethyl acrylate, n-butyl acrylate, hydroxypropyl acrylate, hydroxyethyl acrylate, isobutyl acrylate, and 2-ethylhexyl acrylate. For example, 3-HP may be converted to acrylic acid via a dehydration reaction.

In various embodiments a microorganism is selected from chemolithotrophic bacteria, and more particularly may be Oligotropha carboxidovorans (including strain OM5^{T}, which may alternatively be referred to as strain OM5), Cupriavidus necator, or strain H16 of Cupriavidus necator. Any of the known strains of these species may be utilized as a starting microorganism, as may any of the following species including respective strains thereof—Cupriavidus basilensis, Cupriavidus campinensis, Cupriavidus gilardi, Cupriavidus laharsis, Cupriavidus metallidurans, Cupriavidus oxalaticus, Cupriavidus pauculus, Cupriavidus pinatubonensis, Cupriavidus respiraculi, and Cupriavidus taiwanensis.

More generally, the invention also includes a genetically modified microorganism comprising at least one genetic modification to introduce or increase one or more enzymatic activities selected from the group consisting of phosphoglucose isomerase, inositol-1-phosphate synthase, inositol monophosphatase, myo-inositol dehydrogenase, myo-inosose-2-dehydratase, inositol 2-dehydrogenase, deoxy-D-gluconate isomerase, 5-dehydro-2-deoxygluconokinase, deoxyphophogluconate aldolase, and 3-hydroxy acid dehydrogenase. In various embodiments there may be two or more, three or more, four or more, five or more, and the like, up to all of the noted enzymatic activities, that are provided by the noted at least one genetic modification.

In particular embodiments the genetically modified microorganism may comprise a phosphoglucose isomerase encoded by the pgi gene of E. coli, a inositol-1-phosphate synthase encoded by the ino-1 gene of S. cerevisiae, an inositol monophosphatase encoded by the subB gene of E. coli, a myo-inositol dehydrogenase encoded by the iolG gene of B. subtilis, a myo-inosose-2-dehydratase encoded by the iolE gene of B. subtilis, an inositol 2-dehydrogenase encoded by the iolD gene of B. subtilis, a deoxy-D-gluconate isomerase encoded by the iolB gene of B. subtilis, a 5-dehydro-2-deoxygluconokinase encoded by the iolC gene of B. subtilis, a deoxyphophogluconate aldolase is encoded by the iolJ gene of B. subtilis, and a 3-hydroxy acid dehydrogenase encoded by the ydfG gene of E. coli.

A genetically modified microorganism of the present invention may comprise at least one genetic modification to introduce or increase one or more enzymatic activities provided by amino acid sequences having at least 50%, 60%, 70%, 80%, 85%, 90%, 92%, 95%, 96%, 97%, 98% or 99% sequence identity to one or more amino acid sequences selected from the group consisting of SEQ ID NO:002, SEQ ID NO:004, SEQ ID NO:006, SEQ ID NO:008, SEQ ID NO:010, SEQ ID NO:012, SEQ ID NO:014, SEQ ID NO:016, SEQ ID NO:018, SEQ ID NO:020, SEQ ID NO:022, SEQ ID NO:024, SEQ ID NO:026, and conservatively modified variants thereof. In various embodiments there may be two or more, three or more, four or more, five or more, and the like, up to all of the noted enzymatic activities, that are provided by the noted at least one genetic modification.

Also, a genetically modified microorganism of the invention may comprise at least one genetic modification provided by a polynucleotide comprising a nucleic acid sequence having at least 50%, 60%, 70%, 80%, 85%, 90%, 92%, 95%, 96%, 97%, 98% or 99% sequence identity to one or more nucleic acid sequences from the group consisting of SEQ ID NO:001, SEQ ID NO:003, SEQ ID NO:005, SEQ ID NO:007, SEQ ID NO:009, SEQ ID NO:011, SEQ ID NO:013, SEQ ID NO:015, SEQ ID NO:017, SEQ ID NO:019, SEQ ID NO:021, SEQ ID NO:023, SEQ ID NO:025, and conservatively modified variants thereof. In various embodiments there may be two or more, three or more, four or more, five or more, and the like, up to all of the noted enzymatic activities, that are provided by the noted at least one genetic modification.

A genetically modified microorganism, including any of the above-described genetically modified microorganisms, also may comprise at least one genetic modification to introduce or increase one or more enzymatic activities selected from the group consisting of acrylate:acyl-CoA CoA transferase (such as Hs-acuN of Halomona sp. HTNK1), and acryl-CoA hydratase (such as Hs-acuK of Halomona sp. HTNK1. These enzymatic activities are recognized to catalyze the conversions from acrylate to 3-HP, and are expected to catalyze the reverse, to form acrylate enzymatically from 3-HP produced as described herein, under appropriate conditions. These enzymes may also be used for such conversion to form acrylate in cell-free systems.

The invention also includes a method of converting one or more syngas components, such as carbon dioxide or carbon monoxide and hydrogen, into 3-HP, said method comprising feeding one or more syngas components to a solution comprising a genetically modified microorganism of the invention, as described herein, under suitable fermentation conditions which may be aerobic or anaerobic. In various embodiments of such method the volumetric productivity for 3-HP is at least 1 g/L/hr, or at least 2 g/L/hr. In some embodiments other feedstocks may be provided, including one or more sugars. In other various embodiments of such method the specific productivity for 3-HP is at least 0.005 g/gDCW-hr, 0.05 g/gDCW-hr, 1 g/gDCW-hr, or at least 2 g/gDCW-hr. In some embodiments other feedstocks may be provided, including one or more sugars. For example, the carbon source may have an amount of glucose, sucrose, fructose, dextrose, lactose, glycerol, and/or combinations thereof that is selected from the group consisting of less than about 50%, less than about 40%, less than about 30%, less than about 20%, less than about 10%, less than about 5%, and less than about 1% by weight. In various embodiments, the cell culture comprises an inhibitor of fatty acid synthesis or said microorganism is genetically modified for reduced enzymatic activity in one or more of the microorganism's fatty acid synthesis pathways. The inhibitor of fatty acid synthesis may be selected from the group consisting of thiolactomycin, triclosan, cerulenin, thienodiazaborine, isoniazid, and analogs thereof.

In various embodiments, the invention is directed to a method for producing 3-hydroxypropionic acid (3-HP) comprising: i) combining hydrogen, a carbon source selected from carbon monoxide and carbon dioxide, and a culture of microorganism cells, wherein a) said microorganism cells are genetically transformed to introduce or increase one or more enzymatic activities for conversion of the carbon source to malonate semialdehyde, wherein said enzymatic activities are selected from the group consisting of phosphoglucose isomerase, inositol-1-phosphate synthase, inositol deoxy-D-gluconate isomerase, 5-dehydro-2-deoxygluconokinase, and deoxyphophogluconate aldolase; and b1) said microorganism cells are genetically transformed to introduce or increase enzymatic activity for conversion of malonate semialdehyde to 3-HP, and/or b2) said microorganism cells are capable of producing 3-HP at a rate of at least 1 g/L/hr in the absence of genetic modification for conversion of malonate semialdehyde to 3-HP; and ii) maintaining the combined hydrogen, carbon source, and microorganism cells for a suitable time and under conditions sufficient to produce malonate semialdehyde and convert the malonate semialdehyde to 3-HP. In addition, the invention is directed to methods of producing acrylic acid comprising: i) producing 3-HP according to the methods described above; and ii) converting the 3-HP to acrylic acid. In various embodiments, the invention is directed to a method of producing an acrylic acid-based product comprising; i) producing acrylic acid according to the methods described above; and ii) converting said acrylic acid into an acrylic acid-based product. In various embodiments, the carbon source has a ratio of carbon-14 to carbon-12 of about 1.0×10⁻¹⁴ or greater. The carbon source may have a percentage of petroleum origin selected from less than about 50%, less than about 40%, less than about 30%, less than about 20%, less than about 10%, less than about 5%, less than about 1%, or essentially free of petroleum origin.

In various embodiments, the efficiency of conversion of carbon source to 3-HP is controlled. For example, in various embodiments, the percentage of carbon source converted to 3-HP is selected from greater than 25%, greater than 35%, greater than 45%, greater than 55%, greater than 65%, greater than 75%, greater than 85%, and greater than 95%. Separating and/or purifying 3-HP from cell culture may be achieved by any method, such as by extraction of 3-HP from the culture in the presence of a tertiary amine.

In various embodiments, 3-hydroxypropionic acid according to the invention is essentially free of chemical catalyst. For example, 3HP may be essentially free of chemical catalyst which is a molybdenum and/or vanadium based catalyst. In various embodiments, 3-hydroxypropionic acid has a ratio of carbon-14 to carbon-12 of about 1.0×10⁻¹⁴ or greater. In various embodiments, 3-hydroxypropionic acid according to the invention contains less than about 10% carbon derived from petroleum. In addition, the 3-hydroxypropionic acid may contain a residual amount of organic material related to its method of production. For example, the 3-hydroxypropionic acid may contain a residual amount of organic material in an amount between 1 and 1,000 parts per million of said 3-hydroxypropionic acid.

Further, the invention also includes a method of converting syngas and or one or more sugars to 3-HP using one or more pathways provided herein, such as provided in FIG. 1. For example, the pathway may involve steps 10-12 or step 13 of FIG. 1. In various embodiments of such method the volumetric productivity for 3-HP is at least 1 g/L/hr, or at least 2 g/L/hr.

In addition, the methods of the present invention can also be used to produce downstream compounds derived from 3HP made as provided herein, such as but not limited to polymerized-3-HP (poly-3-HP), acrylic acid, polyacrylic acid (polymerized acrylic acid, in various forms), acrylamide, acrylonitrile, propiolactone, ethyl 3-HP, malonic acid, and 1,3-propanediol. Also, among esters that are formed are methyl acrylate, ethyl acrylate, n-butyl acrylate, hydroxypropyl acrylate, hydroxyethyl acrylate, isobutyl acrylate, and 2-ethylhexyl acrylate. These and/or other acrylic acid and/or other acrylate esters may be combined, including with other compounds, to form various known acrylic acid-based polymers. Numerous approaches may be employed for such downstream conversions, generally falling into enzymatic, catalytic (chemical conversion process using a catalyst), thermal, and combinations thereof (including some wherein a desired pressure is applied to accelerate a reaction). For example, without being limiting, acrylic acid may be made from 3-HP via a dehydration reaction, methyl acrylate may be made from 3-HP via dehydration and esterification, the latter to add a methyl group (such as using methanol), acrylamide may be made from 3-HP via dehydration and amidation reactions, acrylonitrile may be made via a dehydration reaction and forming a nitrile moiety, propriolactone may be made from 3-HP via a ring-forming internal esterification reaction, ethyl-3-HP may be made from 3-HP via esterification with ethanol, malonic acid may be made from 3-HP via an oxidation reaction, and 1,3-propanediol may be made from 3-HP via a reduction reaction. Additionally, it is appreciated that various derivatives of the various derivatives of 3-HP and acrylic acid may be made, such as the various known polymers of acrylic acid and its derivatives. Production of such polymers is considered within the scope of the present invention.

Downstream compounds may in turn be converted to consumer products such as diapers, carpet, paint, and adhesives.

As noted, some of these conversions may be made enzymatically. For example, 3-HP may be converted to 3-HP-CoA, which then may be converted into polymerized 3-HP with an enzyme having polyhydroxyacid synthase activity (EC 2.3.1.-). Also, 1,3-propanediol can be made using polypeptides having oxidoreductase activity or reductase activity (e.g., enzymes in the EC 1.1.1.—class of enzymes). Alternatively, when creating 1,3-propanediol from 3HP, a combination of (1) a polypeptide having aldehyde dehydrogenase activity (e.g., an enzyme from the 1.1.1.34 class) and (2) a polypeptide having alcohol dehydrogenase activity (e.g., an enzyme from the 1.1.1.32 class) can be used. Polypeptides having lipase activity may be used to form esters. Enzymatic reactions such as these may be conducted in vitro, such as using cell-free extracts, or in vivo.

Osmotic shock, sonication, and/or a repeated freeze-thaw cycle followed by filtration and/or centrifugation, among other methods, may be used to produce a cell-free extract from intact cells.

Further as to general processing of a fermentation broth comprising 3-HP, various methods may be practiced to remove biomass and/or 3-HP from the broth. These include centrifugation. Other approaches, such as extraction, distillation, and ion-exchange, in various forms, may be used to separate and/or concentrate the 3-HP. Cell lysis may be conducted, such as described above, as needed to release 3-HP from the cell mass.

The invention also provides a culture system comprising (a) a population of a genetically modified microorganism as described herein and (b) a media comprising nutrients for said population.

The invention also provides a method of making a 3-HP molecule comprising: a. providing one or more genetic modifications to a selected microorganism host cell to obtain all enzymatic conversion steps depicted in FIG. 1 in said host cell; b. providing a supply of carbon dioxide and/or carbon monoxide, and hydrogen to said host cell; and c. culturing the cell under conditions suitable for production of 3-HP from the carbon dioxide and hydrogen.

In various embodiments, the invention comprises methods of making 3-HP using genetically modified microorganism(s) of the invention (such as by the methods described herein), and also methods of making downstream products of 3-HP, including but not limited to polymerized-3-HP (poly-3-HP), acrylic acid, polyacrylic acid, methyl acrylate, acrylamide, acrylonitrile, propiolactone, ethyl 3-HP, malonic acid, and 1,3-propanediol.

The invention provides a method of making 3-HP molecules comprising: a. providing one or more genetic modifications to a selected microorganism host cell to obtain, in a resultant genetically modified microorganism, all numbered enzymatic conversion steps depicted in FIG. 1 (which are also described in Table 1) in said host cell; b. providing a supply of carbon dioxide, and/or carbon monoxide, and hydrogen to said host cell; and c. culturing the cell under conditions suitable for production of 3-HP molecules from the carbon dioxide and hydrogen.

The invention also provides a method of making 3-HP molecules comprising: a. providing one or more genetic modifications to a selected microorganism host cell to obtain, in a resultant genetically modified microorganism, all numbered enzymatic conversion steps depicted in FIG. 1 (which are also described in Table 1) in said genetically modified microorganism; b. providing a supply of a sugar to said genetically modified microorganism; and c. culturing the cell under conditions suitable for production of 3-HP molecules from the sugar.

In various embodiments, the genetically transformed microorganism is further modified to decrease activity in an enzyme selected from the group consisting of lactate dehydrogenase, phosphate acetyltransferase, pyruvate oxidase, pyruvate-formate lyase, and combinations thereof.

The invention also provides a method of making 3-HP molecules in culture vessels such as under industrial bio-production scale and in such systems. Such systems may include the microorganisms according to the invention.

In various embodiments, the invention is directed to a method for producing malonate semialdehyde comprising: a) combining hydrogen, a carbon source selected from carbon monoxide and carbon dioxide, and a culture of microorganism cells, wherein said microorganism cells comprise at least two genetic modifications to introduce or increase one or more enzymatic activities selected from the group consisting of phosphoglucose isomerase, inositol-1-phosphate synthase, inositol monophosphatase, myo-inositol dehydrogenase, myo-inosose-2-dehydratase, inositol 2-dehydrogenase, deoxy-D-gluconate isomerase, 5-dehydro-2-deoxygluconokinase, and deoxyphophogluconate aldolase; and b) maintaining the combined hydrogen, carbon source, and microorganism cells for a suitable time and under conditions sufficient to convert the carbon source to malonate semialdehyde. The invention may also be directed to a method for producing 3-HP comprising: i) producing malonate semialdehyde according to the methods described herein; and ii) maintaining the microorganism cells for a suitable time and under conditions sufficient to convert the malonate semialdehyde to 3-HP; wherein the microorganism cells further comprise a protein that converts malonate semialdehyde to 3-HP. Such proteins may include proteins having 3-hydroxy acid dehydrogenase activity, such as E. coli YdfG, and also, or alternatively, an aldehyde dehydrogenase, a malonyl-CoA synthetase, and a malonyl-CoA reductase (which may be bifunctional or monofunctional, in the latter instance the microorganism also comprising a 3-hydroxy acid dehydrogenase). Acrylic acid may be produced by producing 3-HP as indicated above, and dehydrating the 3-HP to produce acrylic acid, which may in turn be processed into an acrylic-acid based product.

### BRIEF DESCRIPTION OF THE DRAWINGS

FIG. 1 depicts exemplary genetically modified pathways for producing 3-HP from syngas components, according to various embodiments of the invention.

FIG. 2 depicts a method for genetic modification of chromosomal DNA.

The information in any table provided also comprises part of the invention.

### DETAILED DESCRIPTION OF THE INVENTION AND EMBODIMENTS THEREOF

Unless otherwise indicated, all numbers expressing reaction conditions, stoichiometries, sequence similarities, and so forth used in the specification and claims are to be understood as being modified in all instances by the term “about.” Accordingly, unless indicated to the contrary, the numerical parameters set forth in the following specification and attached claims are approximations that may vary depending at least upon the specific analytical technique. Any numerical value inherently contains certain errors necessarily resulting from the standard deviation found in its respective testing measurements.

As used in this specification and the appended claims, the singular forms “a,” “an,” and “the” include plural referents unless the context clearly indicates otherwise. Thus, for example, reference to an “expression vector” includes a single expression vector as well as a plurality of expression vectors, either the same (e.g., the same operon) or different; reference to “microorganism” includes a single microorganism as well as a plurality of microorganisms; and the like.

Unless defined otherwise, all technical and scientific terms used herein have the same meaning as is commonly understood by one of ordinary skill in the art to which this invention belongs. If a definition set forth in this section is contrary to or otherwise inconsistent with a definition set forth in patents, published patent applications, and other publications that are herein incorporated by reference, the definition set forth in this specification prevails over the definition that is incorporated herein by reference.

Certain particular embodiments of the present invention will be described in more detail, including reference to the accompanying figure(s) and table(s). The figure(s) is/are understood to provide representative illustration of the invention and are not limiting in their content or scale. It will be understood by one of ordinary skill in the art that the scope of the invention extends beyond the specific embodiments depicted. This invention also incorporates routine experimentation and optimization of the methods, apparatus, and systems described herein.

There are several groups of bacteria able to utilize the primary components of synthesis gas, mainly H₂ (hydrogen) and CO (carbon monoxide), as sole sources of carbon and energy. One such group is known as chemolithotrophic bacteria, which are able to aerobically utilize carbon dioxide as a carbon source while oxidizing other inorganic sources of energy. This diverse group of bacteria includes ammonia oxidizers, nitrite oxidizers, sulfur oxidizers, iron oxidizers, hydrogen oxidizers, and carbon monoxide oxidizers. Two important aerobic chemolithotrophs include Cupriavidus necator (formerly known as Ralstonia eutropha) and Oligotropha carboxidovorans (formerly known as Pseudomonas carboxidovorans). Cupriavidus necator is able to oxidize hydrogen, while Oligotropha carboxidovorans is able to oxidize carbon monoxide, both in an aerobic environment. Any of the known strains of these species may be utilized as a starting microorganism, as may any of the following species including respective strains thereof—Cupriavidus basilensis, Cupriavidus campinensis, Cupriavidus gilardi, Cupriavidus laharsis, Cupriavidus metallidurans, Cupriavidus oxalaticus, Cupriavidus pauculus, Cupriavidus pinatubonensis, Cupriavidus respiraculi, and Cupriavidus taiwanensis. Another group of syngas utilizers is anaerobic bacteria or archea that are able to fix carbon monoxide through the reductive acetyl-coA pathway.

In some variations, this invention describes and provides metabolic pathways for the production of 3-HP and related products in aerobic chemolithotropes, such as Cupriavidus necator. This group of bacteria can fix carbon dioxide through the Calvin Benson Cycle (CBC), which is the same carbon-fixation cycle used by photosynthetic organisms. In Cupriavidus, this central pathway uses electrons and energy obtained from the oxidation of hydrogen which generates the NADPH and ATP needed for biosynthesis. C. necator is able to obtain reductants and energy needs solely from hydrogen by using two oxygen-tolerant hydrogenases: a soluble hydrogenase and a membrane-bound hydrogenase.

Cupriavidus necator has been characterized to have very high growth rates when grown chemolithotrophically on mixtures of hydrogen and carbon dioxide gases in an aerobic environment (Repaske and Mayer R, “Dense autotrophic cultures of Alcaligenes eutrophus AEM, 32(4), 592-597, 1976). In this species, it is believed (without the present invention being limited to any particular theory) that carbon fixation occurs exclusively through the Calvin Benson Cycle and all cell mass is generated from flux through this pathway. Numerous studies in the literature have shown that productivity through the Calvin Benson Cycle can achieve at least 20 g/L of biomass in 18 hours, or a specific volumetric productivity of approximately 1.34 g/L/hr, under non-optimized conditions and in standard stirred tanks.

The Calvin Benson Cycle is utilized by several chemolithotropic microbes including Oligotropha carboxidovorans and Cupriavidus necator, which can obtain electrons directly from syngas constituents. The megaplasmid pHCG3 of O. carboxidovorans is reported to comprise genes for utilization of CO, CO₂, and/or H₂. Strain H16 of Cupriavidus necator, previously called Ralstonia eutropha, is reported to comprise nucleic acid sequences encoding two hydrogenases and the enzymes of the Calvin Benson Cycle on the megaplasmid pHG1. C. necator has been used commercially to produce polyhydroxyalkanoates (a natural product from this organism) or natural polyester plastics (see, for example, U.S. Pat. Nos. 6,316,262, 6,689,589, 7,081,357, and 7,229,804, incorporated by reference herein for their teachings of microorganism compositions, methods and genes). The genomic sequence of Cupriavidus necator is known and the genomic DNA sequence of Oligotropha carboxidovorans has recently been published (Genome Announcement Genome Sequence of Chemolithotrophic Bacterium Oligotropha carboxidovorans OM5^{T}”, Debarati Paul et al., J. of Bacteriol. 2008:190(5):5531-5532).

The reductive acetyl-CoA cycle is used by many anaerobic microorganisms including methanogens and acetogens. In this cycle, electrons and carbon from CO are used to produce larger molecules. Organisms utilizing this pathway tend to be strict anaerobes and many of the enzymes involved in the cycle itself are very sensitive to the presence of oxygen which inactivates them. This cycle produces acetyl-coA that may then be biologically converted to other products of interest.

The reductive tricarboxylic acid cycle (“TCA”) cycle is used primarily by anaerobic photosynthetic microorganisms. In this cycle CO₂ is fixed into acetyl-CoA by a reverse of the tricarboxylic acid cycle. Many organisms using this fixation cycle are strictly anaerobic and the enzymes that are involved in the cycle are not oxygen tolerant. However, several oxygen-tolerant enzymes involved in this cycle have been characterized.

Thus, several CO₂ fixation pathways such as the above have been characterized. These metabolic pathways use NADH or NADPH as electron carriers for the reduction and fixation of CO₂. In many aerobic photosynthetic organisms such as plants, these carriers are reduced with electrons from water obtained by light-driven reactions. CO and H₂ can be used to reduce these carriers as well. In particular, hydrogenases and CO dehydrogenases are enzymes that can catalyze the transfer of electrons from H₂ and CO, respectively, to NAD⁺ and NADP⁺. Oxygen-tolerant hydrogenases and CO dehydrogenases have been characterized that can carry out these reactions in the presence of oxygen (Bleijlevens et al., “The Auxiliary Protein HypX Provides Oxygen Tolerance to the Soluble [NiFe]-Hydrogenase of Ralstonia eutropha H16 by Way of a Cyanide Ligand to Nickel,” J. Biol. Chem. (2004)279:45, 46686-46691).

Many known bioprocesses utilizing syngas components require anaerobic environments due to the sensitivity of the microorganisms and their enzymes to oxygen. This requirement presents several hurdles and limitations in the bioconversion process. Fixation of CO₂ in these organisms is intimately tied to oxidation of CO or H₂ or to anaerobic cellular respiration.

In an aerobic environment, the reductants NADH and FADH₂ can be used by microorganisms to reduce oxygen to water via aerobic respiration. This allows for the production of energy and ATP via aerobic respiration, independently from CO₂ fixation. An aerobic bioconversion can allow for the microorganism to generate energy for processes other than cellular respiration, such as growth or tolerance to product or feedstock. In addition, the independent production of ATP from CO₂ fixation can allow for the production of higher-energy products from syngas components. In particular, metabolic pathways that utilize ATP to drive the formation of higher-energy products can be achieved.

The variations provided herein related to aerobic processes are consonant with increased microorganism productivities, flexibility of products, product and feedstock tolerance and aerobic respiration, all of which are important issues to be addressed for the successful commercialization of new biofuels and/or bioprocessed chemicals produced from syngas.

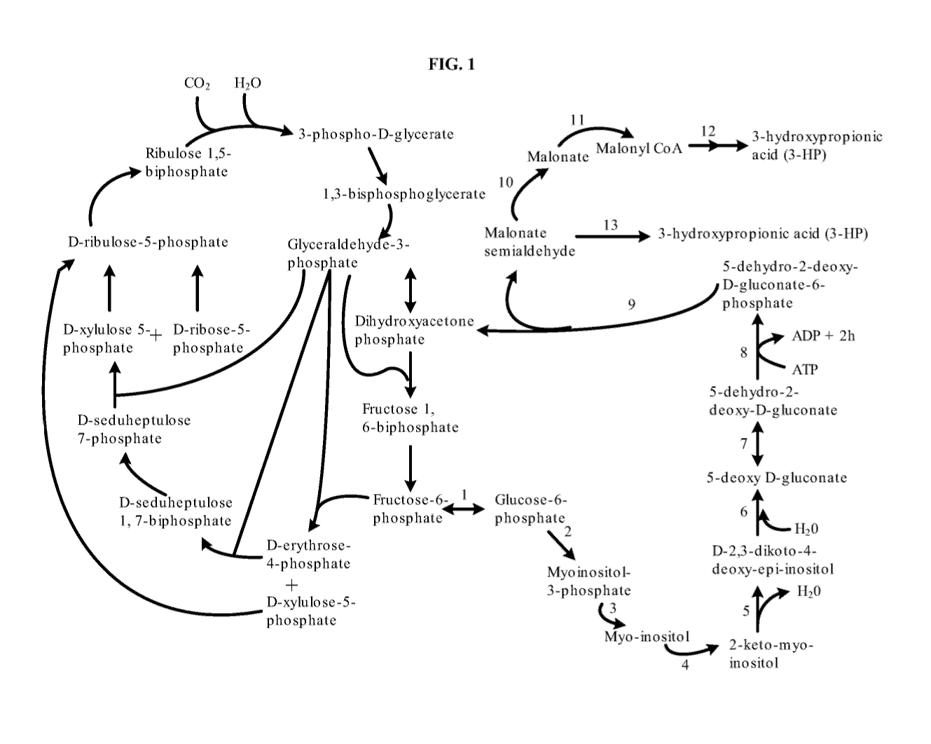

FIG. 1 depicts a metabolic pathway for producing 3-HP from syngas through malonate semialdehyde which may be provided or completed in a microorganism by genetic modification. The malonate semialdehyde is generated from intermediates of the Calvin Benson Cycle, which is depicted on the left side of FIG. 1. It is noted that the FIG. 1 is a summary of the biological reactions that occur. That is, single arrows do not necessarily mean a single enzymatic step, and all of the reactants and products of each step are not necessarily shown. The numbers near arrows in FIG. 1 refer to step numbers as further described in Table 1 herein.

In microorganism genetically modified host cells, and methods and systems comprising such cells, the metabolic reactions depicted in FIG. 1 transpire to yield 3-HP via malonate semialdehyde, which may be derived from carbon dioxide and hydrogen (which in various embodiments are syngas constituents). The latter two compounds enter the Calvin Benson Cycle as shown in FIG. 1, and a later product of the Calvin Benson Cycle, fructose-6-phosphate, is converted to glucose-6-phosphate by a phosphoglucose isomerase. This reaction step begins a side route from the Calvin Benson Cycle that results in the production of dihydroxyacetone phosphate, which may return to and replenish the Calvin Benson Cycle, and malonate semialdehyde, which as depicted in FIG. 1 then is converted to 3-HP such as via the E. coli 3-hydroxy acid dehydrogenase identified as ydfG. Thus, during this series of enzymatic reactions, 3-HP is produced, via several steps, from degradation of myo-inositol which is generated from the glucose-6-phosphate. The net result of this pathway is the generation of 3-HP synthesis and dihydroxyacetone phosphate which can be returned to the Calvin Benson Cycle. Whether the feedstock is syngas or sugars, there can be several entry points for feed components within the metabolic pathway of FIG. 1. As shown in FIG. 1, an alternative route from malonate semialdehyde to 3-HP may proceed through steps 10, 11 and 12. In various embodiments a microorganism may comprise one or both routes to 3-HP, utilizing step 13 and/or steps 10-12.

Table 1 summarizes information regarding the enzymes that catalyze the numbered steps in FIG. 1, including enzyme names, representative genes of species that encode for the specific enzymes, and relevant SEQ ID NOs. for the representative genes and their amino acid products. In various embodiments, this invention provides a method of making a genetically modified microorganism comprising providing to a selected microorganism at least one genetic modification to introduce or increase one or more enzymatic activities selected from the group consisting of phosphoglucose isomerase, inositol-1-phosphate synthase, inositol monophosphatase, myo-inositol dehydrogenase, myo-inosose-2-dehydratase, inositol 2-dehydrogenase, deoxy-D-gluconate isomerase, 5-dehydro-2-deoxygluconokinase, deoxyphosphogluconate aldolase, and 3-hydroxy acid dehydrogenase. In various embodiments, genetic modifications to supply polypeptides providing the enzymatic reactions of steps 10, 11, and/or 12 also are made.

Thus, by providing polypeptides that catalyze enzymatic conversion steps of the myo-inositol pathway (and other steps as depicted in FIG. 1) in a microorganism host cell that comprises Calvin Benson Cycle capability, carbon dioxide and hydrogen, such as from a syngas process, are converted into 3-HP.

In various embodiments, the 3-HP so obtained by microbial bio-synthesis is converted, enzymatically or by other conversion processes, into ‘downstream’ chemicals, including but not limited to polymerized-3-HP (poly-3-HP), acrylic acid, polyacrylic acid, methyl acrylate, acrylamide, acrylonitrile, propiolactone, ethyl 3-HP, malonic acid, and 1,3-propanediol. However, it is noted that in various alternative embodiments malonate semialdehyde is produced and is not converted to 3-HP, but rather is converted to another chemical compound. Thus, there is additional utility to pathways that include steps numbered 1-9 and yield malonate semialdehyde, where further conversion steps yield compounds other than 3-HP from malonate semialdehyde.

However, as to 3-HP downstream product conversions, either enzymatically or by thermal or thermal/catalytic processes, for one example 3-HP is converted via dehydration into acrylic acid. Alternatively by any such processes as are appropriate, polyacrylic acid is formed. Alternatively, by any such processes as are appropriate, 3-HP is converted to 1,3-propanediol, such as by selective direct reduction of the carboxylic acid group. Formation of methyl acrylate from 3-HP may proceed by dehydration and methylation by methods known in the art. Likewise, formation of the other compounds listed above, acrylamide, acrylonitrile, propiolactone, ethyl 3-HP, and malonic acid, may proceed using enzymatic, catalytic, and/or thermal processes in suitable methods, including standard chemical conversion processes.

Standard methodologies (known in the art and further described herein) can be used to generate needed gene expression (or gene disruptions, as described elsewhere herein). In some embodiments, the following enzymatic activities are expressed in C. necator: phosphoglucose isomerase, inositol-1-phosphate synthase, inositol monophosphatase, myo-inositol dehydrogenase, myo-inosose-2-dehydratase, inositol 2-dehydrogenase, deoxy-D-gluconate isomerase, 5-dehydro-2-deoxygluconokinase, deoxyphosphogluconate aldolase, and 3-hydroxy acid dehydrogenase. One or more of these expressed enzymatic activities may be expressed from heterologous (including exogenous) nucleic acid sequences. In various embodiments, the following genes can be employed to encode suitable enzymes to achieve desired levels of expression: E. coli pgi, suhB, and ydfG, S. cerevisiae ino-1, and B. subtilis iolG, iolE, iolD, iolB, and iolC. In various other embodiments, any combination of these genes, and those described in the following paragraphs, and/or functional variants of these, may be provided or employed in a microorganism cell or culture, so as to have the enzymatic activities numbered in FIG. 1 and described in Table 1.

For example, Table 2 shows homologues of most of the proteins of Table 1 in the species C necator and O. carboxidovorans. These homolog sequences are candidates for use and/or further modification so as to obtain a desired enzymatic conversion indicated in FIG. 1 and Table 1 for the indicated steps. Modifications to achieve a suitable activity and a suitable specificity may be made such as by approaches described herein.

Also, as noted a pathway utilizing a malonyl-CoA reductase may be provided. The following section describes alternative approaches to this.

Production Pathway from Malonyl-CoA to 3-HP

In various embodiments the compositions, methods and systems of the present invention involve inclusion of a metabolic production pathway that converts malonyl-CoA to a chemical product of interest. Further as to specific sequences for 3-HP production pathway, malonyl-CoA reductase (mcr) from C. aurantiacus was gene synthesized and codon optimized by the services of DNA 2.0 (See WO 2010/011874, published Jan. 28, 2010, and incorporated by reference for this teaching). The FASTA sequence is shown in (gi|42561982|gb|AAS20429.1| alonyl-CoA reductase (Chloroflexus aurantiacus)).

Mcr has very few sequence homologs in the NCBI data base. Blast searches finds 8 different sequences when searching over the entire protein. Hence development of a pile-up sequences comparison is expected to yield limited information. However, embodiments of the present invention nonetheless may comprise any of these eight sequences, shown herein, which are expected to be but are not yet confirmed to be bi-functional as to this enzymatic activity. Other embodiments may comprise mutated and other variant forms, as well as polynucleotides (including variant forms with conservative and other substitutions), such as those introduced into a selected microorganism to provide or increase 3-HP production therein.

The portion of a CLUSTAL 2.0.11 multiple sequence alignment identifies these eight sequences, as shown in the following table.

TABLE 3

| Reference Nos. | Genus Species |
|---|---|
| gi\|42561982\|gb\|AAS20429.1 | Chloroflexus aurantiacus |
| gi\|163848165\|ref\|YP_001636209 | Chloroflexus aurantiacus J-10-fl |
| gi\|219848167\|ref\|YP_002462600 | Chloroflexus aggregans DSM 9485 |
| gi\|156742880\|ref\|YP_001433009 | Roseiflexus castenholzii DSM 13941 |
| gi\|148657307\|ref\|YP_001277512 | Roseiflexus sp. RS-1 |
| gi\|85708113\|ref\|ZP_01039179.1 | Erythrobacter sp. NAP1 |
| gi\|254282228\|ref\|ZP_04957196.1 | gamma proteobacterium NOR51-B |
| gi\|254513883\|ref\|ZP_05125944.1 | gamma proteobacterium NOR5-3 |
| gi\|119504313\|ref\|ZP_01626393.1 | 3marine gamma proteobacterium |
| | HTCC208 |

Malonyl-CoA may be converted to 3-HP in a microorganism that comprises one or more of the following:

A bi-functional malonyl-CoA reductase, such as may be obtained from Chloroflexus aurantiacus and other microorganism species. By bi-functional in this regard is meant that the malonyl-CoA reductase catalyzes both the conversion of malonyl-CoA to malonate semialdehyde, and of malonate semialdehyde to 3-HP.

A mono-functional malonyl-CoA reductase in combination with a 3-HP dehydrogenase. By mono-functional is meant that the malonyl-CoA reductase catalyzes the conversion of malonyl-CoA to malonate semialdehyde. Particularly, in E. coli Applicants have demonstrated mono-functional malonyl-CoA reductase activity from truncated portions of malonyl-CoA reductase from C. aurantiacus. Thes were constructed by use of PCR primers adjacent, respectively, to nucleotide bases encoding amino acid residues 366 and 1220, and 496 and 1220, of the codon-optimized malonyl-CoA reductase from pTRC-ptrc-mcr-amp. Similar approaches may be provided for other species, such as those described herein.

Any of the above polypeptides may be NADH- or NADPH-dependent, and methods known in the art may be used to convert a particular enzyme to be either form. More particularly, as noted in WO 2002/042418, “any method can be used to convert a polypeptide that uses NADPH as a cofactor into a polypeptide that uses NADH as a cofactor such as those described by others (Eppink et al., J. Mol. Biol., 292 (1): 87-96 (1999), Hall and Tomsett, Microbiology, 146 (Pt 6): 1399-406 (2000), and Dohr et al., Proc. Natl. Acad. Sci., 98 (1): 81-86 (2001)).”

Without being limiting, a bi-functional malonyl-CoA reductase may be selected from the malonyl-CoA reductase of Chloroflexus aurantiacus (such as from ATCC 29365) and other sequences. Also without being limiting, a mono-functional malonyl-CoA reductase may be selected from the malonyl-CoA reductase of Sulfolobus tokodaii (SEQ ID NO:826). As to the malonyl-CoA reductase of C. aurantiacus, that sequence and other species' sequences may also be bi-functional as to this enzymatic activity.

When a mono-functional malonyl-CoA reductase is provided in a microorganism cell, 3-HP dehydrogenase enzymatic activity also may be provided to convert malonate semialdehyde to 3-HP. As shown in the examples, a mono-functional malonyl-CoA reductase may be obtained by truncation of a bi-functional mono-functional malonyl-CoA, and combined in a strain with an enzyme that converts malonate semialdehyde to 3-HP.

Also, it is noted that another malonyl-CoA reductase is known in Metallosphaera sedula (Msed_{—}709, identified as malonyl-CoA reductase/succinyl-CoA reductase).

By providing nucleic acid sequences that encode polypeptides having the above enzymatic activities, a genetically modified microorganism may comprise an effective 3-HP pathway to convert malonyl-CoA to 3-HP in accordance with the embodiments of the present invention.

Other 3-HP pathways, such as those comprising an aminotransferase (see, e.g., WO 2010/011874, published Jan. 28, 2010, and incorporated by reference for this teaching), may also be provided in embodiments of a genetically modified microorganism of the present invention.

The present invention provides for elevated specific and volumetric productivity metrics as to production of a selected chemical product, such as 3-hydroxypropionic acid (3-HP). In various embodiments, production of a chemical product, such as 3-HP, is not linked to growth.

In various embodiments, production of 3-HP, or alternatively one of its downstream products such as described herein, may reach at least 1, at least 2, at least 5, at least 10, at least 20, at least 30, at least 40, and at least 50 g/liter titer, such as by using one of the methods disclosed herein.

As may be realized by appreciation of the advances disclosed herein as they relate to commercial fermentations of selected chemical products, embodiments of the present invention may be combined with other genetic modifications and/or method or system modulations so as to obtain a microorganism (and corresponding method) effective to produce at least 10, at least 20, at least 30, at least 40, at least 45, at least 50, at least 80, at least 100, or at least 120 grams of a chemical product, such as 3-HP, per liter of final (e.g., spent) fermentation broth while achieving this with specific and/or volumetric productivity rates as disclosed herein.

In some embodiments a microbial chemical production event (i.e., a fermentation event using a cultured population of a microorganism) proceeds using a genetically modified microorganism as described herein, wherein the specific productivity is between 0.01 and 0.60 grams of 3-HP produced per gram of microorganism cell on a dry weight basis per hour (g 3-HP/g DCW-hr). In various embodiments the specific productivity is greater than 0.01, greater than 0.05, greater than 0.10, greater than 0.15, greater than 0.20, greater than 0.25, greater than 0.30, greater than 0.35, greater than 0.40, greater than 0.45, or greater than 0.50 g 3-HP/g DCW-hr. Specific productivity may be assessed over a 2, 4, 6, 8, 12 or 24 hour period in a particular microbial chemical production event. More particularly, the specific productivity for 3-HP is between 0.05 and 0.10, 0.10 and 0.15, 0.15 and 0.20, 0.20 and 0.25, 0.25 and 0.30, 0.30 and 0.35, 0.35 and 0.40, 0.40 and 0.45, or 0.45 and 0.50 g 3-HP/g DCW-hr., 0.50 and 0.55, or 0.55 and 0.60 g 3-HP/g DCW-hr. Various embodiments comprise culture systems demonstrating such productivity.

Also, in various embodiments of the present invention the volumetric productivity achieved may be 0.25 g 3-HP per liter per hour (g (chemical product)/L-hr), may be greater than 0.25 g 3-HP/L-hr, may be greater than 0.50 g 3-HP/L-hr, may be greater than 1.0 g 3-HP/L-hr, may be greater than 1.50 g 3-HP/L-hr, may be greater than 2.0 g 3-HP/L-hr, may be greater than 2.50 g 3-HP/L-hr, may be greater than 3.0 g 3-HP/L-hr, may be greater than 3.50 g 3-HP/L-hr, may be greater than 4.0 g 3-HP/L-hr, may be greater than 4.50 g 3-HP/L-hr, may be greater than 5.0 g 3-HP/L-hr, may be greater than 5.50 g 3-HP/L-hr, may be greater than 6.0 g 3-HP/L-hr, may be greater than 6.50 g 3-HP/L-hr, may be greater than 7.0 g 3-HP/L-hr, may be greater than 7.50 g 3-HP/L-hr, may be greater than 8.0 g 3-HP/L-hr, may be greater than 8.50 g 3-HP/L-hr, may be greater than 9.0 g 3-HP/L-hr, may be greater than 9.50 g 3-HP/L-hr, or may be greater than 10.0 g 3-HP/L-hr.

In some embodiments, specific productivity as measured over a 24-hour fermentation (culture) period may be greater than 0.01, 0.05, 0.10, 0.20, 0.5, 1.0, 2.0, 3.0, 4.0, 5.0, 6.0, 7.0, 8.0, 9.0, 10.0, 11.0 or 12.0 grams of chemical product per gram DCW of microorganisms (based on the final DCW at the end of the 24-hour period).

In various aspects and embodiments of the present invention, there is a resulting substantial increase in microorganism specific productivity that advances the fermentation art and commercial economic feasibility of microbial chemical production, such as of 3-HP (but not limited thereto).

Stated in another manner, in various embodiments the specific productivity exceeds (is at least) 0.01 g 3-HP/g DCW-hr, exceeds (is at least) 0.05 g 3-HP/g DCW-hr, exceeds (is at least) 0.10 g 3-HP/g DCW-hr, exceeds (is at least) 0.15 g 3-HP/g DCW-hr, exceeds (is at least) 0.20 g 3-HP/g DCW-hr, exceeds (is at least) 0.25 g 3-HP/g DCW-hr, exceeds (is at least) 0.30 g 3-HP/g DCW-hr, exceeds (is at least) 0.35 g 3-HP/g DCW-hr, exceeds (is at least) 0.40 g 3-HP/g DCW-hr, exceeds (is at least) 0.45 g 3-HP/g DCW-hr, exceeds (is at least) 0.50 g 3-HP/g DCW-hr, exceeds (is at least) 0.60 g 3-HP/g DCW-hr.

More generally, based on various combinations of the genetic modifications described herein, optionally in combination with supplementations and/or other culture conditions described herein, specific productivity values for 3-HP may exceed 0.01 g 3-HP/g DCW-hr, may exceed 0.05 g 3-HP/g DCW-hr, may exceed 0.10 g 3-HP/g DCW-hr, may exceed 0.15 g 3-HP/g DCW-hr, may exceed 0.20 g 3-HP/g DCW-hr, may exceed 0.25 g 3-HP/g DCW-hr, may exceed 0.30 g 3-HP/g DCW-hr, may exceed 0.35 g 3-HP/g DCW-hr, may exceed 0.40 g 3-HP/g DCW-hr, may exceed 0.45 g 3-HP/g DCW-hr, and may exceed 0.50 g or 0.60 3-HP/g DCW-hr. Such specific productivity may be assessed over a 2, 4, 6, 8, 12 or 24 hour period in a particular microbial chemical production event.

The improvements achieved by embodiments of the present invention may be determined by percentage increase in specific productivity, or by percentage increase in volumetric productivity, compared with an appropriate control microorganism lacking the particular genetic modification combinations taught herein (with or without the supplements taught herein, added to a vessel comprising the microorganism population). For particular embodiments and groups thereof, such specific productivity and/or volumetric productivity improvements is/are at least 10, at least 20, at least 30, at least 40, at least 50, at least 100, at least 200, at least 300, at least 400, and at least 500 percent over the respective specific productivity and/or volumetric productivity of such appropriate control microorganism.

The specific methods and teachings of the specification, and/or cited references that are incorporated by reference, may be incorporated into the examples. Also, production of 3-HP, or one of its downstream products such as described herein, may reach at least 1, at least 2, at least 5, at least 10, at least 20, at least 30, at least 40, and at least 50 g/liter titer in various embodiments.

The metrics may be applicable to any of the compositions, e.g., genetically modified microorganisms, methods, e.g., of producing 3-HP or other chemical products, and systems, e.g., fermentation systems utilizing the genetically modified microorganisms and/or methods disclosed herein.

It is appreciated that iterative improvements using the strategies and methods provided herein, and based on the discoveries of the interrelationships of the pathways and pathway portions, may lead to even greater 3-HP production and tolerance and more elevated 3-HP titers at the conclusion of a 3-HP bio-production event.

Any number of strategies may lead to development of a suitable modified enzyme suitable for use in a 3-HP production pathway. With regard to malonyl-CoA-reductase, one may utilize or modify an enzyme such as encoded by the sequences in the table immediately above, to achieve a suitable level of 3-HP production capability in a microorganism strain.

As noted, the use of a malonyl-CoA reductase, such as step 12 in FIG. 1, may be in combination with a 3-hydroxy acid dehydrogenase, stap 13 of FIG. 1, in a selected embodiment.

In some embodiments various other genetic modifications and/or culture system modifications may be made to a selected microorganism to reach an suitable production rate, titer and yield for 3-HP production, such as are enumerated above. The following sections and paragraphs describe many such modifications.

Restricting Fatty Acid Synthesis: Instead of conversion to 3-HP, a possible alternative pathway for malonate semialdehyde involves conversion to malonate, which is converted to malonyl-CoA, which may in certain microorganism species then enter a fatty acid biosynthesis cycle (i.e., elongation by addition of malonyl-CoA). In such circumstances, embodiments may include approaches to decrease any such conversion and ultimately the utilization of malonyl-CoA in fatty acid synthesis. For example, in many microorganism cells the fatty acid synthase system comprises polypeptides that have the following enzymatic activities: malonyl-CoA-acyl carrier protein (ACP) transacylase; β-ketoacyl-ACPsynthase; β-ketoacyl-ACP reductase; β-hydroxyacyl-ACP dehydratase; 3-hydroxyacyl-(acp) dehydratase; and enoyl-acyl carrier protein reductase (enoyl-ACP reductase). In various embodiments nucleic acid sequences that encode temperature-sensitive forms of these polypeptides may be introduced in place of the native enzymes, so that when such genetically modified microorganisms are cultured at elevated temperatures (at which these thermolabile polypeptides become inactivated, partially or completely, due to alterations in protein structure or complete denaturation), so that there may be a decreased conversion of malonate semialdehyde to a fatty acid and a related increase in 3-HP production. In other embodiments other types of genetic modifications may be made to otherwise modulate, such as lower, enzymatic activities of one or more of these polypeptides.

It also is noted that for some embodiments genetic modifications to reduce a microorganism's metabolic conversions of malonate semialdehyde to malonate, and/or of malonate to malonyl-CoA, may similarly provide for suitable increase in 3-HP production from malonate semialdehyde (i.e., via step 13). In various embodiments a result of any such genetic modifications is to shift malonate semialdehyde utilization so that there is a reduced conversion of malonate semialdehyde to fatty acids, overall biomass, and proportionally greater conversion of carbon source to a chemical product such as 3-HP.

As used herein, by the terms “fatty acid synthase,” fatty acid synthase system, and the like, are meant the set of proteins in a microorganism cell that perform the following conversion: condensing a malonyl-CoA or a malonyl-[ACP] with a fatty acyl-CoA or a fatty acyl-[ACP]; reducing the elongated B-ketoacyl[ACP] or B-ketoacyl-CoA; dehydrating the so-formed hydroxyacyl molecule to an enoyl-acyl[ACP] or enoyl-acyl-CoA, and then reducing this to a so-elongated fatty acyl-[ACP] or fatty acyl-CoA. This can then go through further elongations until a sufficient length for further reactions described herein. This reaction generally starts with a C4 or greater alkyl molecule.

One enzyme, enoyl(acyl carrier protein) reductase (EC No. 1.3.1.9, also referred to as enoyl-ACP reductase) is a key enzyme for fatty acid biosynthesis from malonyl-CoA. In Escherichia coli this enzyme, FabI, is encoded by the gene fabI (See “Enoyl-Acyl Carrier Protein (fabI) Plays a Determinant Role in Completing Cycles of Fatty Acid Elongation in Escherichia coli,” Richard J. Heath and Charles O. Rock, J. Biol. Chem. 270:44, pp. 26538-26543 (1995), incorporated by reference for its discussion of fabI and the fatty acid synthase system).

The present invention may utilize a microorganism that is provided with a nucleic acid sequence (polynucleotide) that encodes a polypeptide having enoyl-ACP reductase enzymatic activity that may be modulated during a fermentation event. For example, a nucleic acid sequence encoding a temperature-sensitive enoyl-ACP reductase may be provided in place of the native enoyl-ACP reductase, so that an elevated culture temperature results in reduced enzymatic activity, which then results in a shifting utilization of malonate semialdehyde to 3-HP. At such elevated temperature the enzyme is considered non-permissive, as is the temperature. One such sequence is a mutant temperature-sensitive fabI (fabI^{TS}) of E. coli, which has a mutation of C to T at position 722 (See Bergler, H., Hogenauer, G., and Turnowsky, F., J. Gen. Microbiol. 138:2093-2100 (1992). This mutant demonstrates relatively normal activity at reduced temperature, such as 30 C, and becomes non-permissive, likely through denaturation and inactivation, at elevated temperature, such that when cultured at 37 to 42 C a microorganism only comprising this temperature-sensitive mutant as its enoyl-ACP reductase will produce substantially less fatty acids and phospholipids. This leads to decreased or no growth, and provide for increased utilization of malonyl-CoA for 3-HP production when a suitable protein, such as a malonyl-CoA enzyme is provided. The same or a similar mutation may be made in the corresponding enoyl-ACP reductase of a species disclosed herein, evaluated and evolved as needed using known methodologies, so as to obtain a suitable temperature-sensitive protein that may be used in various embodiments of the invention.

It is appreciated that nucleic acid and amino acid sequences for enoyl-ACP reductase in species other than E. coli are readily obtained by conducting homology searches in known genomics databases, such as BLASTN and BLASTP. Approaches to obtaining homologues in other species and functional equivalent sequences are described herein. Accordingly, it is appreciated that the present invention may be practiced by one skilled in the art for many microorganism species of commercial interest.

Other approaches than a temperature-sensitive enoyl-ACP reductase may be employed as known to those skilled in the art, such as, but not limited to, replacing a native enoyl-ACP or enoyl-coA reductase with a nucleic acid sequence that includes an inducible promoter for this enzyme, so that an initial induction may be followed by no induction, thereby decreasing enoyl-ACP or enoyl-coA reductase enzymatic activity after a selected cell density is attained. For example, a promoter may be induced (such as with isopropyl-μ-D-thiogalactopyranoiside (IPTG)) during a first phase of a method herein, and after the IPTG is exhausted, removed or diluted out the second step, of reducing enoyl-ACP reductase enzymatic activity, may begin. Other approaches may be applied to control enzyme expression and activity such as are described herein and/or known to those skilled in the art.

While enoyl-CoA reductase is considered an important enzyme of the fatty acid synthase system, genetic modifications may be made to any combination of the polynucleotides (nucleic acid sequences) encoding the polypeptides exhibiting the enzymatic activities of this system, such as are listed herein. For example, FabB, β-ketoacyl-acyl carrier protein synthase I, is an enzyme in E. coli that is essential for growth and the biosynthesis of both saturated and unsaturated fatty acids. Inactivation of FabB results in the inhibition of fatty acid elongation and diminished cell growth as well as eliminating a futile cycle that recycles the malonate moiety of malonyl-ACP back to acetyl-CoA. FabF, β-ketoacyl-acyl carrier protein synthase II, is required for the synthesis of saturated fatty acids and the control membrane fluidity in cells. Both enzymes are inhibited by cerulenin.

It is reported that overexpression of FabF results in diminished fatty acid biosynthesis. It is proposed that FabF outcompetes FabB for association with FabD, malonyl-CoA:ACP transacylase. The association of FabB with FabD is required for the condensation reaction that initiates fatty acid elongation. (See Microbiological Reviews, September 1993, p. 522-542 Vol. 57, No. 3; K. Magnuson et al., “Regulation of Fatty Acid Biosynthesis in Escherichia coli,” American Society for Microbiology; W. Zha et al., “Improving cellular malonyl-CoA level in Escherichia coli via metabolic engineering,” Metabolic Engineering 11 (2009) 192-198). An alternative to genetic modification to reduce such fatty acid synthase enzymes is to provide into a culture system a suitable inhibitor of one or more such enzymes. This approach may be practiced independently or in combination with the genetic modification approach. Inhibitors, such as cerulenin, thiolactomycin, thienodiazaborine, isoniazid, triclosan, and analogs thereof (this list not limiting) or genetic modifications directed to reduce activity of enzymes encoded by one or more of the fatty acid synthetase system genes may be employed, singly or in combination.

In certain compositions, methods and systems of the present invention the reduction of enzymatic activity of enoyl-ACP reductase (or, more generally, of the fatty acid synthase system) is made to occur after a sufficient cell density of a genetically modified microorganism is attained. This bi-phasic culture approach balances a desired quantity of catalyst, in the cell biomass which supports a particular production rate, with yield, which may be partly attributed to having less carbon be directed to cell mass after the enoyl-ACP reductase activity (and/or activity of other enzymes of the fatty acid synthase system) is/are reduced. This results in a shifting net utilization of malonate semialdehyde, thus providing for greater carbon flux to a desired chemical product, namely 3-HP.

In various embodiments of the present invention the specific productivity is elevated and this results in overall rapid and efficient microbial fermentation methods and systems. In various embodiments the volumetric productivity also is substantially elevated.

In various embodiments a genetically modified microorganism comprises a metabolic pathway that includes conversion of carbon dioxide and/or carbon monoxide to malonate semialdehyde and then to a desired chemical product, 3-hydroxypropionic acid (3-HP). This is viewed as quite advantageous for commercial 3-HP production economics and is viewed as an advance having clear economic benefit.

By “means for modulating” the conversion of malonate semialdehyde to fatty acyl-ACP or fatty acyl-coA molecules, and to fatty acid molecules, is meant any one of the following: 1) providing in a microorganism cell at least one polynucleotide that encodes at least one polypeptide having activity of one of the fatty acid synthase system enzymes (such as recited herein), wherein the polypeptide so encoded has (such as by mutation and/or promoter substitution, etc., to lower enzymatic activity), or may be modulated to have (such as by temperature sensitivity, inducible promoter, etc.) a reduced enzymatic activity; 2) providing to a vessel comprising a microorganism cell or population an inhibitor that inhibits enzymatic activity of one or more of the fatty acid synthase system enzymes (such as recited herein), at a dosage effective to reduce enzymatic activity of one or more of these enzymes. These means may be provided in combination with one another. When a means for modulating involves a conversion, during a fermentation event, from a higher to a lower activity of the fatty acid synthetase system, such as by increasing temperature of a culture vessel comprising a population of genetically modified microorganism comprising a temperature-sensitive fatty acid synthetase system polypeptide (e.g., enoyl-ACP reductase), or by adding an inhibitor, there are conceived two modes—one during which there is higher activity, and a second during which there is lower activity, of such fatty acid synthetase system. During the lower activity mode, a shift to greater utilization of malonate semialdehyde to a selected chemical product may proceed.

Once the modulation is in effect to decrease the noted enzymatic activity(ies), each respective enzymatic activity so modulated may be reduced by at least 10, at least 20, at least 30, at least 40, at least 50, at least 60, at least 70, at least 80, or at least 90 percent compared with the activity of the native, non-modulated enzymatic activity (such as in a cell or isolated). Similarly, the conversion of malonate semialdehyde to fatty acyl-ACP or fatty acyl-coA molecules may be reduced by at least 10, at least 20, at least 30, at least 40, at least 50, at least 60, at least 70, at least 80, or at least 90 percent compared with such conversion in a non-modulated cell or other system. Likewise, the conversion of malonate semialdehyde to fatty acid molecules may be reduced by at least 10, at least 20, at least 30, at least 40, at least 50, at least 60, at least 70, at least 80, or at least 90 percent compared with such conversion in a non-modulated cell or other system.

In some aspects, compositions, methods and systems of the present invention shift utilization of malonate semialdehyde in a genetic modified microorganism, which comprises at least one enzyme of the fatty acid synthase system, such as enoyl-acyl carrier protein reductase (enoyl-ACP reductase) or enoyl-coenzyme A reductase (enoyl-coA reductase), β-ketoacyl-ACP synthase or β-ketoacyl-coAsynthase malonyl-CoA-ACP, and may further comprise at least one genetic modification of nucleic acid sequence encoding carbonic anhydrase to increase bicarbonate levels in the microorganism cell and/or a supplementation of its culture medium with bicarbonate and/or carbonate, and may further comprise one or more genetic modifications to increase enzymatic activity of one or more of acetyl-CoA carboxylase and NADPH-dependent transhydrogenase. More generally, addition of carbonate and/or bicarbonate may be used to increase bicarbonate levels in a fermentation broth.

Additional Genetic Modifications for Improved 3-HP Production: Further to the last paragraph, a number of additional genetic medications may be made to increase 3-HP production in a selected microorganism. These may be made in any combination, including with the above-described modifications. As may be appropriate, various nucleic acid sequences are codon-optimized for the selected microorganism.

Some embodiments of the invention additionally may comprise a genetic modification to increase the availability of the cofactor NADPH, which can increase the NADPH/NADP+ ratio as may be desired. Non-limiting examples for such genetic modification are pgi (E.C. 5,3.1.9, in a mutated form), pntAB (E.C. 1.6.1.2), overexpressed, gapA (E.C. 1.2.1.12):gapN (E.C. 1.2.1.9, from Streptococcus mutans) substitution/replacement, and disrupting or modifying a soluble transhydrogenase such as sthA (E.C. 1.6.1.2), and/or genetic modifications of one or more of zwf (E.C. 1.1.1.49), gnd (E.C. 1.1.1.44), and edd (E.C. 4.2.1.12). Sequences of these genes are available at www.metacyc.org. Also, the sequences for the genes and encoded proteins for the E. coli gene names shown in Tables 6A, 6B, and 7 are provided in U.S. Provisional Patent Application No. 61/246,141, incorporated herein in its entirety and for such sequences, and also are available at www.ncbi.gov as well as www.metacyc.org or www.ecocyc.org.

In some embodiments, genetic modifications may be provided that specifically increase tolerance to 3-HP. In this regard, PCT publication WO/2010/011874, published Jan. 28, 2010 (application PCT/US2009/051607) is incorporated by reference herein for its teachings of various genetic modifications and culture system supplements that may be provided to increase microorganism tolerance to 3-HP. The teachings so incorporated include those directed to the species C. necator.

The invention also comprises a method of making a genetically modified microorganism comprising providing to a selected microorganism at least one genetic modification to decrease one or more enzymatic activities selected from the group consisting of fatty acid synthase, polyhydroxybutyrate polymerase, acetoacetyl-coA reductase, acetyl-coA acetyltransferase, NADH dependant 3-hydroxypropionate dehydrogenase, 3-hydroxypropionate synthetase, malonate semialdehyde dehydrogenase, acetylating malonate semialdehyde dehydrogenase, methylmalonate semialdehyde dehydrogenase, acetylating methylmalonate semialdehyde dehydrogenase and 3-hydroxypropionaldehyde dehydrogenase. In various embodiments there may be two or more, three or more, four or more, or all of the noted enzymatic activities, that are provided by the noted at least one genetic modification. A genetically modified microorganism, including any of the above-described genetically modified microorganisms, also may comprise at least one genetic modification to introduce or increase one or more enzymatic activities selected from the group consisting of NADPH dependant 3-hydroxypropionate dehydrogenase, malonyl-coA reductase, malonate semialdehyde dehydrogenase and malonyl-coA synthetase. In various embodiments there may be two or more, three or more, four or more, or all of the noted enzymatic activities, that are provided by the noted at least one genetic modification.

Also, for all nucleic acid and amino acid sequences provided herein, it is appreciated that conservatively modified variants of these sequences are included, and are within the scope of the invention in its various embodiments. Functionally equivalent nucleic acid and amino acid sequences (functional variants), which may include conservatively modified variants as well as more extensively varied sequences, which are well within the skill of the person of ordinary skill in the art, and microorganisms comprising these, also are within the scope of various embodiments of the invention, as are methods and systems comprising such sequences and/or microorganisms. Also, as used herein, the language “sufficiently homologous” refers to proteins or portions thereof that have amino acid sequences that include a minimum number of identical or equivalent amino acid residues when compared to an amino acid sequence of the amino acid sequences listed in Table 1 such that the protein or portion thereof is able to participate in the respective reaction shown in FIG. 1 and described in Table 1. To determine whether a particular protein or portion thereof is sufficiently homologous may be determined by an assay of enzymatic activity, such as those commonly known in the art. In various embodiments, nucleic acid sequences encoding sufficiently homologous proteins or portions thereof are within the scope of the invention. More generally, nucleic acids sequences that encode a particular amino acid sequence employed in the invention may vary due to the degeneracy of the genetic code, and nonetheless fall within the scope of the invention. Table 4 provides a summary of similarities among amino acids, upon which conservative and less conservative substitutions may be based, and also various codon redundancies that reflect this degeneracy.

More generally, the invention encompasses various genetic modifications and evaluations to certain microorganisms. The scope of the invention is not meant to be limited to such microorganism species, but to be generally applicable to a wide range of suitable microorganisms. As the genomes of various species become known, features of the present invention easily may be applied to an ever-increasing range of suitable microorganisms. Further, given the relatively low cost of genetic sequencing, the genetic sequence of a species of interest may readily be determined to make application of aspects of the present invention more readily obtainable (based on the ease of application of genetic modifications to an organism having a known genomic sequence). More generally, a microorganism used for the present invention may be selected from bacteria, cyanobacteria, filamentous fungi, and yeasts.

More particularly, based on the various criteria described herein, suitable microbial hosts for the bio-production of 3-HP provided herein generally may include, but are not limited to, any gram negative organisms such as E. coli, Oligotropha carboxidovorans, or Pseudomononas sp.; any gram positive microorganism, for example Bacillus subtilis, Lactobaccilus sp. or Lactococcus sp.; any yeast, for example Saccharomyces cerevisiae, Pichia pastoris or Pichia stipitis; and other groups of microbial species. Species and other phylogenic identifications herein are according to the classification known to a person skilled in the art of microbiology.

More particularly, suitable microbial hosts for the bio-production of 3-HP generally include, but are not limited to, members of the genera Clostridium, Zymomonas, Escherichia, Salmonella, Rhodococcus, Pseudomonas, Bacillus, Lactobacillus, Enterococcus, Alcaligenes, Klebsiella, Paenibacillus, Arthrobacter, Corynebacterium, Brevibacterium, Pichia, Candida, Hansenula and Saccharomyces.

Hosts that may be particularly of interest include: Oligotropha carboxidovorans (such as strain OM5^{T}), Escherichia coli, Cupriavidus necator, formerly Alcaligenes eutrophus, Ralstonia eutropha) (such as strain DSM542), Bacillus licheniformis, Paenibacillus macerans, Rhodococcus erythropolis, Pseudomonas putida, Lactobacillus plantarum, Enterococcus faecium, Enterococcus gallinarium, Enterococcus faecalis, Bacillus subtilis and Saccharomyces cerevisiae. Also, any of the known strains of these species may be utilized as a starting microorganism, as may any of the following species including respective strains thereof—Cupriavidus basilensis, Cupriavidus campinensis, Cupriavidus gilardi, Cupriavidus laharsis, Cupriavidus metallidurans, Cupriavidus oxalaticus, Cupriavidus pauculus, Cupriavidus pinatubonensis, Cupriavidus respiraculi, and Cupriavidus taiwanensis.

In some embodiments, the recombinant microorganism is a gram-negative bacterium. In some embodiments, the recombinant microorganism is selected from the genera Zymomonas, Escherichia, Pseudomonas, Alcaligenes, and Klebsiella. In some embodiments, the recombinant microorganism is selected from the species Escherichia coli, Cupriavidus necator, Oligotropha carboxidovorans, and Pseudomonas putida. In some embodiments, the recombinant microorganism is an E. coli strain.

In some embodiments, the recombinant microorganism is a gram-positive bacterium. In some embodiments, the recombinant microorganism is selected from the genera Clostridium, Salmonella, Rhodococcus, Bacillus, Lactobacillus, Enterococcus, Paenibacillus, Arthrobacter, Corynebacterium, and Brevibacterium. In some embodiments, the recombinant microorganism is selected from the species Bacillus licheniformis, Paenibacillus macerans, Rhodococcus erythropolis, Lactobacillus plantarum, Enterococcus faecium, Enterococcus gallinarium, Enterococcus faecalis, and Bacillus subtilis. In particular embodiments, the recombinant microorganism is a B. subtilis strain.

In some embodiments, the recombinant microorganism is a yeast. In some embodiments, the recombinant microorganism is selected from the genera Pichia, Candida, Hansenula and Saccharomyces. In particular embodiments, the recombinant microorganism is Saccharomyces cerevisiae.

Also, in some embodiments the microorganism comprises an endogenous 3-HP production pathway (which may, in some such embodiments, be enhanced), whereas in other embodiments the microorganism does not comprise a 3-HP production pathway, but is provided with one or more nucleic acid sequences encoding polypeptides having enzymatic activity or activities to complete a pathway, described herein, resulting in production of 3-HP. In some embodiments, the particular sequences disclosed herein, or conservatively modified variants thereof, are provided to a selected microorganism, such as selected from one or more of the species and groups of species or other taxonomic groups listed above.

Notwithstanding the discussion on the use of such chemolithotrophs and syngas components for carbon and energy sources, pathways and polynucleotides encoding polypeptides exhibiting enzymatic activity of such pathways described herein also may be used (introduced) in species, methods and systems that use sugars or other suitable substrates as the carbon and energy source.

Suitable substrates include glucose, fructose, and sucrose, as well as mixtures of any of these sugars. Sucrose may be obtained from feedstocks such as sugar cane, sugar beets, cassaya, and sweet sorghum. Glucose and dextrose may be obtained through saccharification of starch-based feedstocks including grains such as corn, wheat, rye, barley, and oats.

Suitable substrates may generally include, but are not limited to, monosaccharides such as glucose and fructose, oligosaccharides such as lactose or sucrose, polysaccharides such as starch or cellulose or mixtures thereof and unpurified mixtures from renewable feedstocks such as cheese whey permeate, cornsteep liquor, sugar beet molasses, and barley malt. In addition, methylotrophic organisms are known to utilize a number of other carbon containing compounds such as methylamine, glucosamine and a variety of amino acids for metabolic activity. For example, methylotrophic yeast are known to utilize the carbon from methylamine to form trehalose or glycerol (Bellion et al., Microb. Growth Cl Compd. [Int. Symp.], 7th (1993), 415-32. Editor(s): Murrell, J. Collin; Kelly, Don P. Publisher: Intercept, Andover, UK). Similarly, various species of Candida will metabolize alanine or oleic acid (Sulter et al., Arch. Microbiol. 153:485-489 (1990)). Hence it is contemplated that the source of carbon utilized in embodiments of the present invention may encompass a wide variety of carbon-containing substrates.

In addition, fermentable sugars may be obtained from cellulosic and lignocellulosic biomass through processes of pretreatment and saccharification, as described, for example, in U.S. Patent App. Pub. No. US20070031918A1, which is incorporated by reference herein for its teachings. Biomass refers to any cellulosic or lignocellulosic material and includes materials comprising cellulose, and optionally further comprising hemicellulose, lignin, starch, oligosaccharides and/or monosaccharides. Biomass may also comprise additional components, such as proteins and/or lipids. Biomass may be derived from a single source, or biomass can comprise a mixture derived from more than one source; for example, biomass could comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste. Examples of biomass include, but are not limited to, corn grain, corn cobs, crop residues such as corn husks, corn stover, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers and animal manure. Any such biomass may be used in a bio-production method or system to provide a carbon source.

The ability to genetically modify a host cell is essential for the production of any genetically modified (recombinant) microorganism. The mode of gene transfer technology may be by electroporation, conjugation, transduction, or natural transformation. A broad range of host conjugative plasmids and drug resistance markers are available. The cloning vectors are tailored to the host organisms based on the nature of antibiotic resistance markers that can function in that host.

For various embodiments of the invention the genetic manipulations may be described to include various genetic manipulations, including those directed to change regulation of, and therefore ultimate activity of, an enzyme or enzymatic activity of an enzyme identified in any of the respective pathways. Such genetic modifications may be directed to transcriptional, translational, and post-translational modifications that result in a change of enzyme activity and/or selectivity under selected and/or identified culture conditions and/or to provision of additional nucleic acid sequences such as to increase copy number and/or mutants of an enzyme related to 3-HP production. Specific methodologies and approaches to achieve such genetic modification are well known to one skilled in the art, and include, but are not limited to: increasing expression of an endogenous genetic element; decreasing functionality of a repressor gene; introducing a heterologous genetic element; increasing copy number of a nucleic acid sequence encoding a polypeptide catalyzing an enzymatic conversion step to produce 3-HP; mutating a genetic element to provide a mutated protein to increase specific enzymatic activity; over-expressing; under-expressing; over-expressing a chaperone; knocking out a protease; altering or modifying feedback inhibition; providing an enzyme variant comprising one or more of an impaired binding site for a repressor and/or competitive inhibitor; knocking out a repressor gene; evolution, selection and/or other approaches to improve mRNA stability as well as use of plasmids having an effective copy number and promoters to achieve an effective level of improvement. Random mutagenesis may be practiced to provide genetic modifications that may fall into any of these or other stated approaches. The genetic modifications further broadly fall into additions (including insertions), deletions (such as by a mutation) and substitutions of one or more nucleic acids in a nucleic acid of interest. In various embodiments a genetic modification results in improved enzymatic specific activity and/or turnover number of an enzyme. Without being limited, changes may be measured by one or more of the following: K_{M}; K_{cat}; and K_{avidity}.

In various embodiments, to function more efficiently, a microorganism may comprise one or more gene deletions. For example, in E. coli, the genes encoding the pyruvate kinase (pfkA and pfkB), lactate dehydrogenase (ldhA), phosphate acetyltransferase (pta), pyruvate oxidase (poxB), and pyruvate-formate lyase (pflB) may be disrupted, including deleted. Such gene disruptions, including deletions, are not meant to be limiting, and may be implemented in various combinations in various embodiments. Gene deletions may be accomplished by mutational gene deletion approaches, and/or starting with a mutant strain having reduced or no expression of one or more of these enzymes, and/or other methods known to those skilled in the art. Gene deletions may be effectuated by any of a number of known specific methodologies, including but not limited to the RED/ET methods using kits and other reagents sold by Gene Bridges (Gene Bridges GmbH, Dresden, Germany, www.genebridges.com). The homologous recombination method using Red/ET recombination, is known to those of ordinary skill in the art and described in U.S. Pat. Nos. 6,355,412 and 6,509,156, issued to Stewart et al. and incorporated by reference herein for its teachings of this method. Material and kits for such method are available from Gene Bridges (Gene Bridges GmbH, Heidelberg (formerly Dresden), Germany, <<www.genebridges.com>>), and the method proceeded by following the manufacturer's instructions. The method replaces the target gene by a selectable marker via homologous recombination performed by the recombinase from λ-phage. The host organism expressing k-red recombinase is transformed with a linear DNA product coding for a selectable marker flanked by the terminal regions (generally ˜50 bp, and alternatively up to about ˜300 bp) homologous with the target gene or promoter sequence.

Further, for 3-HP production, such genetic modifications may be chosen and/or selected for to achieve a higher flux rate through certain enzymatic conversion steps within the respective 3-HP production pathway and so may affect general cellular metabolism in fundamental and/or major ways. Another method enabling genetic modification of chromosomal DNA including gene deletion in C. necator involves integration of counterselectable markers, such as Bacillus sacB markers which confer sensitivity to sucrose, via suicide plasmids. These methods are well known in the art.

As used herein, the term “gene disruption,” or grammatical equivalents thereof (and including “to disrupt enzymatic function,” “disruption of enzymatic function,” and the like), is intended to mean a genetic modification to a microorganism that renders the encoded gene product as having a reduced polypeptide activity compared with polypeptide activity in or from a microorganism cell not so modified. The genetic modification can be, for example, deletion of the entire gene, deletion or other modification of a regulatory sequence required for transcription or translation, deletion of a portion of the gene which results in a truncated gene product (e.g., enzyme) or by any of various mutation strategies that reduces activity (including to no detectable activity level) the encoded gene product. A disruption may broadly include a deletion of all or part of the nucleic acid sequence encoding the enzyme, and also includes, but is not limited to other types of genetic modifications, e.g., introduction of stop codons, frame shift mutations, introduction or removal of portions of the gene, and introduction of a degradation signal, those genetic modifications affecting mRNA transcription levels and/or stability, and altering the promoter or repressor upstream of the gene encoding the enzyme.

In some embodiments, a gene disruption is taken to mean any genetic modification to the DNA, mRNA encoded from the DNA, and the amino acid sequence resulting there from that results in reduced polypeptide activity. Many different methods can be used to make a cell having reduced polypeptide activity. For example, a cell can be engineered to have a disrupted regulatory sequence or polypeptide-encoding sequence using common mutagenesis or knock-out technology. See, e.g., Methods in Yeast Genetics (1997 edition), Adams et al., Cold Spring Harbor Press (1998). One particularly useful method of gene disruption is complete gene deletion because it reduces or eliminates the occurrence of genetic reversions in the genetically modified microorganisms of the invention. Accordingly, a disruption of a gene whose product is an enzyme thereby disrupts enzymatic function. Alternatively, antisense technology can be used to reduce the activity of a particular polypeptide. For example, a cell can be engineered to contain a cDNA that encodes an antisense molecule that prevents a polypeptide from being translated. The term “antisense molecule” as used herein encompasses any nucleic acid molecule or nucleic acid analog (e.g., peptide nucleic acids) that contains a sequence that corresponds to the coding strand of an endogenous polypeptide. An antisense molecule also can have flanking sequences (e.g., regulatory sequences). Thus, antisense molecules can be ribozymes or antisense oligonucleotides. A ribozyme can have any general structure including, without limitation, hairpin, hammerhead, or axhead structures, provided the molecule cleaves RNA. Further, gene silencing can be used to reduce the activity of a particular polypeptide.

The term “reduction” or “to reduce” when used in such phrase and its grammatical equivalents are intended to encompass a complete elimination of such conversion(s). The term “heterologous DNA,” “heterologous nucleic acid sequence,” and the like as used herein refers to a nucleic acid sequence wherein at least one of the following is true: (a) the sequence of nucleic acids is foreign to (i.e., not naturally found in) a given host microorganism; (b) the sequence may be naturally found in a given host microorganism, but in an unnatural (e.g., greater than expected) amount; or (c) the sequence of nucleic acids comprises two or more subsequences that are not found in the same relationship to each other in nature. For example, regarding instance (c), a heterologous nucleic acid sequence that is recombinantly produced will have two or more sequences from unrelated genes arranged to make a new functional nucleic acid. Embodiments of the present invention may result from introduction of an expression vector into a host microorganism, wherein the expression vector contains a nucleic acid sequence coding for an enzyme that is, or is not, normally found in a host microorganism. With reference to the host microorganism's genome prior to the introduction of the heterologous nucleic acid sequence, then, the nucleic acid sequence that codes for the enzyme is heterologous (whether or not the heterologous nucleic acid sequence is introduced into that genome). Also, when the genetic modification of a gene product, i.e., an enzyme, is referred to herein, including the claims, it is understood that the genetic modification is of a nucleic acid sequence, such as or including the gene, that normally encodes the stated gene product, i.e., the enzyme. The term “heterologous” is intended to include the term “exogenous” as the latter term is generally used in the art.

Bio-production media, which is used in embodiments of the present invention with genetically modified microorganisms, must contain suitable carbon substrates for the intended metabolic pathways. As described hereinbefore, suitable carbon substrates include carbon monoxide, carbon dioxide, and various monomeric and oligomeric sugars.

In some variations, one or more carbon sources should be minimized or excluded from the bio-production media. In the case of auxotrophic fermentations of C. necator, minimal medias may be employed, as supplementation of certain carbon sources, particularly amino acids, can cause metabolism of these compounds rather than hydrogen and carbon dioxide. Also, it is known in the art that syngas streams may contain toxic components such as heavy metals and aromatic tars. In some embodiments, metals and tars are minimized in the bio-production media.

In some embodiments, genetic elements that provide increased tolerance to, or detoxify, tars and similar components are identified and thereafter incorporated into a microorganism of interest for 3-HP production. One technique that may precisely and rapidly identify such genomic elements is the SCALES technique, described in U.S. Patent Publication US2006/0084098, published Apr. 20, 2006, and incorporated by reference herein for the teachings of the technique of that application. Inter alia, this technique may be applied to identify genetic elements that provide increased tolerance to toxic components associated with a particular syngas from a particular source, or may be applied more broadly.

Typically cells are grown at a temperature in the range of about 25° C. to about 40° C. in an appropriate medium, as well as up to 70° C. for thermophilic microorganisms. Suitable growth media for embodiments of the present invention are common commercially prepared media such as Luria Bertani (LB) broth, M9 minimal media, Sabouraud Dextrose (SD) broth, Yeast medium (YM) broth (Ymin) yeast synthetic minimal media and minimal media as described herein, such as M9 minimal media. Other defined or synthetic growth media may also be used, and the appropriate medium for growth of the particular microorganism will be known by one skilled in the art of microbiology or bio-production science. In various embodiments a minimal media may be developed and used that does not comprise, or that has a low level of addition (e.g., less than 0.2, or less than one, or less than 0.05 percent) of one or more of yeast extract and/or a complex derivative of a yeast extract, e.g., peptone, tryptone, etc.

Suitable pH ranges for the bio-production are between pH 3.0 to pH 10.0, where pH 6.0 to pH 8.0 is a typical pH range for the initial condition. However, the actual culture conditions for a particular embodiment are not meant to be limited by these pH ranges.

Bio-productions may be performed under aerobic, microaerobic, or anaerobic conditions, with or without agitation. The operation of cultures and populations of microorganisms to achieve aerobic, microaerobic and anaerobic conditions are known in the art, and dissolved oxygen levels of a liquid culture comprising a nutrient media and such microorganism populations may be monitored to maintain or confirm a desired aerobic, microaerobic or anaerobic condition. When syngas is used as a feedstock, aerobic conditions may be utilized (although not required to practice this invention). When sugars are used, anaerobic, aerobic or microaerobic conditions can be implemented in various embodiments.

The amount of 3-HP produced in a bio-production media generally can be determined using a number of methods known in the art, for example, high performance liquid chromatography (HPLC), gas chromatography (GC), or GC/Mass Spectroscopy (MS).

Any of the recombinant microorganisms as described and/or referred to above may be introduced into an industrial bio-production system where the microorganisms convert a carbon source into 3-HP, and optionally in various embodiments also to one or more downstream compounds of 3-HP in a commercially viable operation. The bio-production system includes the introduction of such a recombinant microorganism into a bioreactor vessel, with a carbon source substrate and bio-production media suitable for growing the recombinant microorganism, and maintaining the bio-production system within a suitable temperature range (and dissolved oxygen concentration range if the reaction is aerobic or microaerobic) for a suitable time to obtain a desired conversion of a portion of the substrate molecules to 3-HP. Industrial bio-production systems and their operation are well-known to those skilled in the arts of chemical engineering and bioprocess engineering. The following paragraphs provide an overview of the methods and aspects of industrial systems that may be used for the bio-production of 3-HP.

In various embodiments, syngas components or sugars are provided to a microorganism, such as in an industrial system comprising a reactor vessel in which a defined media (such as a minimal salts media including but not limited to M9 minimal media, potassium sulfate minimal media, yeast synthetic minimal media and many others or variations of these), an inoculum of a microorganism providing an embodiment of the biosynthetic pathway(s) taught herein, and the carbon source may be combined. The carbon source enters the cell and is catabolized by well-known and common metabolic pathways to yield common metabolic intermediates, including phosphoenolpyruvate (PEP). (See Molecular Biology of the Cell, 3^{rd} Ed., B. Alberts et al. Garland Publishing, New York, 1994, pp. 42-45, 66-74, incorporated by reference for the teachings of basic metabolic catabolic pathways for sugars; Principles of Biochemistry, 3^{rd} Ed., D. L. Nelson & M. M. Cox, Worth Publishers, New York, 2000, pp. 527-658, incorporated by reference for the teachings of major metabolic pathways; and Biochemistry, 4^{th} Ed., L. Stryer, W. H. Freeman and Co., New York, 1995, pp. 463-650, also incorporated by reference for the teachings of major metabolic pathways.).

Further to types of industrial bio-production, various embodiments of the present invention may employ a batch type of industrial bioreactor. A classical batch bioreactor system is considered “closed” meaning that the composition of the medium is established at the beginning of a respective bio-production event and not subject to artificial alterations and additions during the time period ending substantially with the end of the bio-production event. Thus, at the beginning of the bio-production event the medium is inoculated with the desired organism or organisms, and bio-production is permitted to occur without adding anything to the system. Typically, however, a “batch” type of bio-production event is batch with respect to the addition of carbon source and attempts are often made at controlling factors such as pH and oxygen concentration. In batch systems the metabolite and biomass compositions of the system change constantly up to the time the bio-production event is stopped. Within batch cultures cells moderate through a static lag phase to a high growth log phase and finally to a stationary phase where growth rate is diminished or halted. If untreated, cells in the stationary phase will eventually die. Cells in log phase generally are responsible for the bulk of production of a desired end product or intermediate.

A variation on the standard batch system is the fed-batch system. Fed-batch bio-production processes are also suitable when practicing embodiments of the present invention and comprise a typical batch system with the exception that the nutrients, including the substrate, are added in increments as the bio-production progresses. Fed-batch systems are useful when catabolite repression is apt to inhibit the metabolism of the cells and where it is desirable to have limited amounts of substrate in the media. Measurement of the actual nutrient concentration in fed-batch systems may be measured directly, such as by sample analysis at different times, or estimated on the basis of the changes of measurable factors such as pH, dissolved oxygen and the partial pressure of waste gases such as CO₂. Batch and fed-batch approaches are common and well known in the art and examples may be found in Thomas D. Brock in Biotechnology: A Textbook of Industrial Microbiology, Second Edition (1989) Sinauer Associates, Inc., Sunderland, Mass., Deshpande, Mukund V., Appl. Biochem. Biotechnol., 36:227, (1992), and Biochemical Engineering Fundamentals, 2^{nd} Ed. J. E. Bailey and D. F. 011 is, McGraw Hill, New York, 1986, herein incorporated by reference for general instruction on bio-production, which as used herein may be aerobic, microaerobic, or anaerobic.

Although embodiments of the present invention may be performed in batch mode, or in fed-batch mode, it is contemplated that the invention would be adaptable to continuous bio-production methods. Continuous bio-production is considered an “open” system where a defined bio-production medium is added continuously to a bioreactor and an equal amount of conditioned media is removed simultaneously for processing. Continuous bio-production generally maintains the cultures within a controlled density range where cells are primarily in log phase growth. Two types of continuous bioreactor operation include a chemostat, wherein fresh media is fed to the vessel while simultaneously removing an equal rate of the vessel contents. The limitation of this approach is that cells are lost and high cell density generally is not achievable. In fact, typically one can obtain much higher cell density with a fed-batch process. Another continuous bioreactor utilizes perfusion culture, which is similar to the chemostat approach except that the stream that is removed from the vessel is subjected to a separation technique which recycles viable cells back to the vessel. This type of continuous bioreactor operation has been shown to yield significantly higher cell densities than fed-batch and can be operated continuously. Continuous bio-production is particularly advantageous for industrial operations because it has less down time associated with draining, cleaning and preparing the equipment for the next bio-production event. Furthermore, it is typically more economical to continuously operate downstream unit operations, such as distillation, than to run them in batch mode.

Continuous bio-production allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by media turbidity, is kept constant. Methods of modulating nutrients and growth factors for continuous bio-production processes as well as techniques for maximizing the rate of product formation are well known in the art of industrial microbiology and a variety of methods are detailed by Brock, supra.

It is contemplated that cells may be immobilized on an inert scaffold as whole cell catalysts and subjected to suitable bio-production conditions for 3-HP production, or be cultured in liquid media in a vessel, such as a culture vessel. Thus, embodiments used in such processes, and in bio-production systems using these processes, include a population of genetically modified microorganisms of the present invention, a culture system comprising such population in a media comprising nutrients for the population, and methods of making 3-HP and thereafter, a downstream product of 3-HP.

Embodiments of the invention include methods of making 3-HP in a bio-production system, some of which methods may include obtaining 3-HP after such bio-production event. For example, a method of making 3-HP may comprise: providing to a culture vessel a media comprising suitable nutrients; providing to the culture vessel an inoculum of a genetically modified microorganism comprising genetic modifications described herein such that the microorganism produces 3-HP from syngas and/or a sugar molecule; and maintaining the culture vessel under suitable conditions for the genetically modified microorganism to produce 3-HP. In various embodimentsof the invention the volume of the aqueous medium (also referred to as culture medium) is selected from greater than 5 mL, greater than 100 mL, greater than 0.5 L, greater than 1 L, greater than 2 L, greater than 10 L, greater than 250 L, greater than 1000 L, greater than 10,000 L, greater than 50,000 L, greater than 100,000 L or greater than 200,000 L, uch as when the volume of the aqueous medium is greater than 250 L and contained within a steel vessel.

Separation and Purification of the Chemical Product 3-HP

When 3-HP is the chemical product, the 3-HP may be separated and purified by the approaches described in the following paragraphs, taking into account that many methods of separation and purification are known in the art and the following disclosure is not meant to be limiting. Osmotic shock, sonication, homogenization, and/or a repeated freeze-thaw cycle followed by filtration and/or centrifugation, among other methods, such as pH adjustment and heat treatment, may be used to produce a cell-free extract from intact cells. Any one or more of these methods also may be employed to release 3-HP from cells as an extraction step.

Further as to general processing of a bio-production broth comprising 3-HP, various methods may be practiced to remove biomass and/or separate 3-HP from the culture broth and its components. Methods to separate and/or concentrate the 3-HP include centrifugation, filtration, extraction, chemical conversion such as esterification, distillation (which may result in chemical conversion, such as dehydration to acrylic acid, under some reactive-distillation conditions), crystallization, chromatography, and ion-exchange, in various forms. Additionally, cell rupture may be conducted as needed to release 3-HP from the cell mass, such as by sonication, homogenization, pH adjustment or heating. 3-HP may be further separated and/or purified by methods known in the art, including any combination of one or more of centrifugation, liquid-liquid separations, including extractions such as solvent extraction, reactive extraction, two-phase aqueous extraction and two-phase solvent extraction, membrane separation technologies, distillation, evaporation, ion-exchange chromatography, adsorption chromatography, reverse phase chromatography and crystallization. Any of the above methods may be applied to a portion of a bio-production broth (i.e., a fermentation broth, whether made under aerobic, anaerobic, or microaerobic conditions), such as may be removed from a bio-production event gradually or periodically, or to the broth at termination of a bio-production event. Conversion of 3-HP to downstream products, such as described herein, may proceed after separation and purification, or, such as with distillation, thin-film evaporation, or wiped-film evaporation optionally also in part as a separation means.

For various of these approaches, one may apply a counter-current strategy, or a sequential or iterative strategy, such as multi-pass extractions. For example, a given aqueous solution comprising 3-HP may be repeatedly extracted with a non-polar phase comprising an amine to achieve multiple reactive extractions.

When a culture event (fermentation event) is at a point of completion, the spent broth may transferred to a separate tank, or remain in the culture vessel, and in either case the temperature may be elevated to at least 60° C. for a minimum of one hour in order to kill the microorganisms. (Alternatively, other approaches to killing the microorganisms may be practiced.) By spent broth is meant the final liquid volume comprising the initial nutrient media, cells grown from the microorganism inoculum (and possibly including some original cells of the inoculum), 3-HP, and optionally liquid additions made after providing the initial nutrient media, such as periodic additions to provide additional carbon source, etc. It is noted that the spent broth may comprise organic acids other than 3-HP, such as for example acetic acid and/or lactic acid.

A centrifugation step may then be practiced to filter out the biomass solids (e.g., microorganism cells). This may be achieved in a continuous or batch centrifuge, and solids removal may be at least about 80%, 85%, 90%, or 95% in a single pass, or cumulatively after two or more serial centrifugations.

An optional step is to polish the centrifuged liquid through a filter, such as microfiltration or ultrafiltration, or may comprise a filter press or other filter device to which is added a filter aid such as diatomaceous earth. Alternative or supplemental approaches to this and the centrifugation may include removal of cells by a flocculent, where the cells floc and are allowed to settle, and the liquid is drawn off or otherwise removed. A flocculent may be added to a fermentation broth after which settling of material is allowed for a time, and then separations may be applied, including but not limited to centrifugation.

After such steps, a spent broth comprising 3-HP and substantially free of solids is obtained for further processing. By “substantially free of solids” is meant that greater than 98%, 99%, or 99.5% of the solids have been removed.

In various embodiments this spent broth comprises various ions of salts, such as Na, Cl, SO₄, and PO₄. In some embodiments these ions may be removed by passing this spent broth through ion exchange columns, or otherwise contacting the spent broth with appropriate ion exchange material. Here and elsewhere in this document, “contacting” is taken to mean a contacting for the stated purpose by any way known to persons skilled in the art, such as, for example, in a column, under appropriate conditions that are well within the ability of persons of ordinary skill in the relevant art to determine. As but one example, these may comprise sequential contacting with anion and cation exchange materials (in any order), or with a mixed anion/cation material. This demineralization step should remove most such inorganic ions without removing the 3-HP. This may be achieved, for example, by lowering the pH sufficiently to protonate 3-HP and similar organic acids so that these acids are not bound to the anion exchange material, whereas anions, such as Cl and SO₄, that remain charged at such pH are removed from the solution by binding to the resin. Likewise, positively charged ions are removed by contacting with cation exchange material. Such removal of ions may be assessed by a decrease in conductivity of the solution. Such ion exchange materials may be regenerated by methods known to those skilled in the art.

In some embodiments, the spent broth (such as but not necessarily after the previous demineralization step) is subjected to a pH elevation, after which it is passed through an ion exchange column, or otherwise contacted with an ion exchange resin, that comprises anionic groups, such as amines, to which organic acids, ionic at this pH, associate. Other organics that do not so associate with amines at this pH (which may be over 6.5, over 7.5, over 8.5, over 9.5, over 10.5, or higher pH) may be separated from the organic acids at this stage, such as by flushing with an elevated pH rinse. Thereafter elution with a lower pH and/or elevated salt content rinse may remove the organic acids. Eluting with a gradient of decreasing pH and/or increasing salt content rinses may allow more distinct separation of 3-HP from other organic acids, thereafter simplifying further processing.

This latter step of anion-exchange resin retention of organic acids may be practiced before or after the demineralization step. However, the following two approaches are alternatives to the anion-exchange resin step.

A first alternative approach comprises reactive extraction (a form of liquid-liquid extraction) as exemplified in this and the following paragraphs. The spent broth, which may be at a stage before or after the demineralization step above, is combined with a quantity of a tertiary amine such as Alamine-336® (Cognis Corp., Cincinnati, Ohio USA) at low pH. Co-solvents for the Alamine-336 or other tertiary amine may be added and include, but are not limited to benzene, carbon tetrachloride, chloroform, cyclohexane, disobutyl ketone, ethanol, #2 fuel oil, isopropanol, kerosene, n-butanol, isobutanol, octanol, and n-decanol that increase the partition coefficient when combined with the amine. After appropriate mixing a period of time for phase separation transpires, after which the non-polar phase, which comprises 3-HP associated with the Alamine-336 or other tertiary amine, is separated from the aqueous phase.

When a co-solvent is used that has a lower boiling point than the 3-HP/tertiary amine, a distilling step may be used to remove the co-solvent, thereby leaving the 3-HP-tertiary amine complex in the non-polar phase.

Whether or not there is such a distillation step, a stripping or recovery step may be used to separate the 3-HP from the tertiary amine. An inorganic salt, such as ammonium sulfate, sodium chloride, or sodium carbonate, or a base such as sodium hydroxide or ammonium hydroxide, is added to the 3-HP/tertiary amine to reverse the amine protonation reaction, and a second phase is provided by addition of an aqueous solution (which may be the vehicle for provision of the inorganic salt). After suitable mixing, two phases result and this allows for tertiary amine regeneration and re-use, and provides the 3-HP in an aqueous solution. Alternatively, hot water may also be used without a salt or base to recover the 3HP from the amine.

In the above approach the phase separation and extraction of 3-HP to the aqueous phase can serve to concentrate the 3-HP. It is noted that chromatographic separation of respective organic acids also can serve to concentrate such acids, such as 3-HP. In similar approaches other suitable, non-polar amines, which may include primary, secondary and quaternary amines, may be used instead of and/or in combination with a tertiary amine.

A second alternative approach is crystallization. For example, the spent broth (such as free of biomass solids) may be contacted with a strong base such as ammonium hydroxide, which results in formation of an ammonium salt of 3-HP. This may be concentrated, and then ammonium-3-HP crystals are formed and may be separated, such as by filtration, from the aqueous phase. Once collected, ammonium-3-HP crystals may be treated with an acid, such as sulfuric acid, so that ammonium sulfate is regenerated, so that 3-HP and ammonium sulfate result.

Also, various aqueous two-phase extraction methods may be utilized to separate and/or concentrate a desired chemical product from a fermentation broth or later-obtained solution. It is known that the addition of polymers, such as dextran and glycol polymers, such as polyethylene glycol (PEG) and polypropylene glycol (PPG) to an aqueous solution may result in formation of two aqueous phases. In such systems a desired chemical product may segregate to one phase while cells and other chemicals partition to the other phase, thus providing for a separation without use of organic solvents. This approach has been demonstrated for some chemical products, but challenges associated with chemical product recovery from a polymer solution and low selectivities are recognized (See “Extractive Recovery of Products from Fermentation Broths,” Joong Kyun Kim et al., Biotechnol. Bioprocess Eng., 1999(4)1-11, incorporated by reference for all of its teachings of extractive recovery methods).

Various substitutions and combinations of the above steps and processes may be made to obtain a relatively purified 3-HP solution. Also, methods of separation and purification disclosed in U.S. Pat. No. 6,534,679, issued Mar. 18, 2003, and incorporated by reference herein for such methods disclosures, may be considered based on a particular processing scheme. Also, in some culture events periodic removal of a portion of the liquid volume may be made, and processing of such portion(s) may be made to recover the 3-HP, including by any combination of the approaches disclosed above.

As noted, solvent extraction is another alternative. This may use any of a number of and/or combinations of solvents, including alcohols, esters, ketones, and various organic solvents. Without being limiting, after phase separation a distillation step or a secondary extraction may be employed to separate 3-HP from the organic phase.

The following published resources are incorporated by reference herein for their respective teachings to indicate the level of skill in these relevant arts, and as needed to support a disclosure that teaches how to make and use methods of industrial bio-production of 3-HP, and also industrial systems that may be used to achieve such conversion with any of the recombinant microorganisms of the present invention (Biochemical Engineering Fundamentals, 2^{nd} Ed. J. E. Bailey and D. F. Ollis, McGraw Hill, New York, 1986, entire book for purposes indicated and Chapter 9, pp. 533-657 in particular for biological reactor design; Unit Operations of Chemical Engineering, 5^{th} Ed., W. L. McCabe et al., McGraw Hill, New York 1993, entire book for purposes indicated, and particularly for process and separation technologies analyses; Equilibrium Staged Separations, P. C. Wankat, Prentice Hall, Englewood Cliffs, N.J. USA, 1988, entire book for separation technologies teachings)

The methods of the present invention can also be used to produce “downstream” compounds derived from 3HP made as provided herein, such as polymerized-3-HP (poly-3-HP), acrylic acid, polyacrylic acid (polymerized acrylic acid, in various forms), methyl acrylate, acrylamide, acrylonitrile, propiolactone, ethyl 3-HP, malonic acid, and 1,3-propanediol. Numerous approaches may be employed for such downstream conversions, generally falling into enzymatic, catalytic (chemical conversion process using a catalyst), thermal, and combinations thereof (including some wherein a desired pressure is applied to accelerate a reaction). For example, without being limiting, acrylic acid may be made from 3-HP via a dehydration reaction, which may be achieved by a number of commercial methodologies including via a distillation process, which may be part of the separation regime and which may include an acid and/or a metal ion as catalyst. More broadly, incorporated herein for its teachings of conversion of 3-HP, and other β-hydroxy carbonyl compounds, to acrylic acid and other related downstream compounds, is U.S. Patent Publication No. 20070219390 A1, published Sep. 20, 2007. This publication lists numerous catalysts and provides examples of conversions, which are specifically incorporated herein. The following section provides more details of various processing methods for a number of these downstream products, including consumer products.

Conversion of 3-HP to Acrylic Acid and Downstream Products

As discussed herein, various embodiments described herein are related to production of a particular chemical product, 3-hydroxypropionic acid (3-HP). This organic acid, 3-HP, may be converted to various other products having industrial uses, such as but not limited to acrylic acid, esters of acrylic acid, and other chemicals obtained from 3-HP, referred to as “downstream products.” Under some approaches the 3-HP may be converted to acrylic acid, acrylamide, and/or other downstream chemical products, in some instances the conversion being associated with the separation and/or purification steps. Many conversions to such downstream products are described herein. The methods of the invention include steps to produce downstream products of 3-HP. As a C₃ building block, 3-HP offers much potential in a variety of chemical conversions to commercially important intermediates, industrial end products, and consumer products. For example, 3-HP may be converted to acrylic acid, acrylates (e.g., acrylic acid salts and esters), 1,3-propanediol, malonic acid, ethyl-3-hydroxypropionate, ethyl ethoxy propionate, propiolactone, acrylamide, or acrylonitrile.

For example, methyl acrylate may be made from 3-HP via dehydration and esterification, the latter to add a methyl group (such as using methanol); acrylamide may be made from 3-HP via dehydration and amidation reactions; acrylonitrile may be made via a dehydration reaction and forming a nitrile moiety; propriolactone may be made from 3-HP via a ring-forming internal esterification reaction (eliminating a water molecule); ethyl-3-HP may be made from 3-HP via esterification with ethanol; malonic acid may be made from 3-HP via an oxidation reaction; and 1,3-propanediol may be made from 3-HP via a reduction reaction. Also, acrylic acid, first converted from 3-HP by dehydration, may be esterified with appropriate compounds to form a number of commercially important acrylate-based esters, including but not limited to methyl acrylate, ethyl acrylate, methyl acrylate, 2-ethylhexyl acrylate, butyl acrylate, and lauryl acrylate. Alternatively, 3HP may be esterified to form an ester of 3HP and then dehydrated to form the acrylate ester.

Additionally, 3-HP may be oligomerized or polymerized to form poly(3-hydroxypropionate) homopolymers, or co-polymerized with one or more other monomers to form various co-polymers. Because 3-HP has only a single stereoisomer, polymerization of 3-HP is not complicated by the stereo-specificity of monomers during chain growth. This is in contrast to (S)-2-Hydroxypropanoic acid (also known as lactic acid), which has two (D, L) stereoisomers that must be considered during its polymerizations.

As will be further described, 3-HP can be converted into derivatives starting (i) substantially as the protonated form of 3-hydroxypropionic acid; (ii) substantially as the deprotonated form, 3-hydroxypropionate; or (iii) as mixtures of the protonated and deprotonated forms. Generally, the fraction of 3-HP present as the acid versus the salt will depend on the pH, the presence of other ionic species in solution, temperature (which changes the equilibrium constant relating the acid and salt forms), and to some extent pressure. Many chemical conversions may be carried out from either of the 3-HP forms, and overall process economics will typically dictate the form of 3-HP for downstream conversion.

Also, as an example of a conversion during separation, 3-HP in an amine salt form, such as in the extraction step herein disclosed using Alamine 336 as the amine, may be converted to acrylic acid by contacting a solution comprising the 3-HP amine salt with a dehydration catalyst, such as aluminum oxide, at an elevated temperature, such as 170 to 180 C, or 180 to 190 C, or 190 to 200 C, and passing the collected vapor phase over a low temperature condenser. Operating conditions, including 3-HP concentration, organic amine, co-solvent (if any), temperature, flow rates, dehydration catalyst, and condenser temperature, are evaluated and improved for commercial purposes. Conversion of 3-HP to acrylic acid is expected to exceed at least 80 percent, or at least 90 percent, in a single conversion event. The amine may be re-used, optionally after clean-up. Other dehydration catalysts, as provided herein, may be evaluated. It is noted that U.S. Pat. No. 7,186,856 discloses data regarding this conversion approach, albeit as part of an extractive salt-splitting conversion that differs from the teachings herein. However, U.S. Pat. No. 7,186,856 is incorporated by reference for its methods, including extractive salt-splitting, the latter to further indicate the various ways 3-HP may be extracted from a microbial fermentation broth.

Further as to embodiments in which the chemical product being synthesized by the microorganism host cell is 3-HP, made as provided herein and optionally purified to a selected purity prior to conversion, the methods of the present invention can also be used to produce “downstream” compounds derived from 3-HP, such as polymerized-3-HP (poly-3-HP), acrylic acid, polyacrylic acid (polymerized acrylic acid, in various forms), methyl acrylate, acrylamide, acrylonitrile, propiolactone, ethyl 3-HP, malonic acid, and 1,3-propanediol. Numerous approaches may be employed for such downstream conversions, generally falling into enzymatic, catalytic (chemical conversion process using a catalyst), thermal, and combinations thereof (including some wherein a desired pressure is applied to accelerate a reaction).

As noted, an important industrial chemical product that may be produced from 3-HP is acrylic acid. Chemically, one of the carbon-carbon single bonds in 3-HP must undergo a dehydration reaction, converting to a carbon-carbon double bond and rejecting a water molecule. Dehydration of 3-HP in principle can be carried out in the liquid phase or in the gas phase. In some embodiments, the dehydration takes place in the presence of a suitable homogeneous or heterogeneous catalyst. Suitable dehydration catalysts are both acid and alkaline catalysts. Following dehydration, an acrylic acid-containing phase is obtained and can be purified where appropriate by further purification steps, such as by distillation methods, extraction methods, or crystallization methods, or combinations thereof.

Making acrylic acid from 3-HP via a dehydration reaction may be achieved by a number of commercial methodologies including via a distillation process, which may be part of the separation regime and which may include an acid and/or a metal ion as catalyst. More broadly, incorporated herein for its teachings of conversion of 3-HP, and other β-hydroxy carbonyl compounds, to acrylic acid and other related downstream compounds, is U.S. Patent Publication No. 2007/0219390 A1, published Sep. 20, 2007, now abandoned. This publication lists numerous catalysts and provides examples of conversions, which are specifically incorporated herein. Also among the various specific methods to dehydrate 3-HP to produce acrylic acid is an older method, described in U.S. Pat. No. 2,469,701 (Redmon). This reference teaches a method for the preparation of acrylic acid by heating 3-HP to a temperature between 130 and 190° C., in the presence of a dehydration catalyst, such as sulfuric acid or phosphoric acid, under reduced pressure. U.S. Patent Publication No. 2005/0222458 A1 (Craciun et al.) also provides a process for the preparation of acrylic acid by heating 3-HP or its derivatives. Vapor-phase dehydration of 3-HP occurs in the presence of dehydration catalysts, such as packed beds of silica, alumina, or titania. These patent publications are incorporated by reference for their methods relating to converting 3-HP to acrylic acid.

The dehydration catalyst may comprise one or more metal oxides, such as Al₂O₃, SiO₂, or TiO₂. In some embodiments, the dehydration catalyst is a high surface area Al₂O₃ or a high surface area silica wherein the silica is substantially SiO₂. High surface area for the purposes of the invention means a surface area of at least about 50, 75, 100 m²/g, or more. In some embodiments, the dehydration catalyst may comprise an aluminosilicate, such as a zeolite.

For example, including as exemplified from such incorporated references, 3-HP may be dehydrated to acrylic acid via various specific methods, each often involving one or more dehydration catalysts. One catalyst of particular apparent value is titanium, such as in the form of titanium oxide, TiO(2). A titanium dioxide catalyst may be provided in a dehydration system that distills an aqueous solution comprising 3-HP, wherein the 3-HP dehydrates, such as upon volatilization, converting to acrylic acid, and the acrylic acid is collected by condensation from the vapor phase.

As but one specific method, an aqueous solution of 3-HP is passed through a reactor column packed with a titanium oxide catalyst maintained at a temperature between 170 and 190 C and at ambient atmospheric pressure. Vapors leaving the reactor column are passed over a low temperature condenser, where acrylic acid is collected. The low temperature condenser may be cooled to 30 C or less, 2 C or less, or at any suitable temperature for efficient condensation based on the flow rate and design of the system. Also, the reactor column temperatures may be lower, for instance when operating at a pressure lower than ambient atmospheric pressure. It is noted that Example 1 of U.S. Patent Publication No. 2007/0219390, published Sep. 20, 2007, now abandoned, provides specific parameters that employs the approach of this method. As noted, this publication is incorporated by reference for this teaching and also for its listing of catalysts that may be used in a 3-HP to acrylic acid dehydration reaction.

Further as to dehydration catalysts, the following table summarizes a number of catalysts (including chemical classes) that may be used in a dehydration reaction from 3-HP (or its esters) to acrylic acid (or acrylate esters). Such catalysts, some of which may be used in any of solid, liquid or gaseous forms, may be used individually or in any combination. This listing of catalysts in Table 5, below is not intended to be limiting, and many specific catalysts not listed may be used for specific dehydration reactions. Further without being limiting, catalyst selection may depend on the solution pH and/or the form of 3-HP in a particular conversion, so that an acidic catalyst may be used when 3-HP is in acidic form, and a basic catalyst may be used when the ammonium salt of 3-HP is being converted to acrylic acid. Also, some catalysts may be in the form of ion exchange resins.

TABLE 5
Dehydration Catalysts

| Catalyst by | |
|---|---|
| Chemical Class | Non-limiting Examples |
| Acids (including | H₂SO₄,HCl, titanic acids, metal oxide hydrates, metal sulfates |
| weak and strong) | (MSO₄,. where M = Zn, Sn, Ca, Ba, Ni, Co, or other transition metals), |
| | metal oxide sulfates, metal phosphates (e.g., M₃,(PO₄)₂, where M = Ca, |
| | Ba), metal phosphates, metal oxide phosphates, carbon (e.g., transition |
| | metals on a carbon support), mineral acids, carboxylic acids, salts thereof, |
| | acidic resins, acidic zeolites, clays, SiO₂/H₃PO₄, fluorinated Al₂O₃, |
| | Nb₂O₃/PO₅⁻³, Nb₂O₃/SO₄⁻², Nb₂O₅H₂O, phosphotungstic acids, |
| | phosphomolybdic acids, silicomolybdic acids, silicotungstic acids, carbon |
| | dioxide |
| Bases (including | NaOH, ammonia, polyvinylpyridine, metal hydroxides, Zr(OH)₄, and |
| weak and strong) | substituted amines |
| Oxides (generally | TiO₂, ZrO₂, Al₂O₃, SiO₂, ZnO₂, SnO₂, WO₃, MnO₂, Fe₂O₃, V₂O₅ |
| metal oxides) | |

As to another specific method using one of these catalysts, concentrated sulfuric acid and an aqueous solution comprising 3-HP are separately flowed into a reactor maintained at 150 to 165° C. at a reduced pressure of 100 mm Hg. Flowing from the reactor is a solution comprising acrylic acid. A specific embodiment of this method, disclosed in Example 1 of US2009/0076297, incorporated by reference herein, indicates a yield of acrylic acid exceeding 95 percent.

Based on the wide range of possible catalysts and knowledge in the art of dehydration reactions of this type, numerous other specific dehydration methods may be evaluated and implemented for commercial production.

The dehydration of 3-HP may also take place in the absence of a dehydration catalyst. For example, the reaction may be run in the vapor phase in the presence of a nominally inert packing such as glass, ceramic, a resin, porcelain, plastic, metallic or brick dust packing and still form acrylic acid in reasonable yields and purity. The catalyst particles can be sized and configured such that the chemistry is, in some embodiments, mass-transfer-limited or kinetically limited. The catalyst can take the form of powder, pellets, granules, beads, extrudates, and so on. When a catalyst support is optionally employed, the support may assume any physical form such as pellets, spheres, monolithic channels, etc. The supports may be co-precipitated with active metal species; or the support may be treated with the catalytic metal species and then used as is or formed into the aforementioned shapes; or the support may be formed into the aforementioned shapes and then treated with the catalytic species.

A reactor for dehydration of 3-HP may be engineered and operated in a wide variety of ways. The reactor operation can be continuous, semi-continuous, or batch. It is perceived that an operation that is substantially continuous and at steady state is advantageous from operations and economics perspectives. The flow pattern can be substantially plug flow, substantially well-mixed, or a flow pattern between these extremes. A “reactor” can actually be a series or network of several reactors in various arrangements.

For example, without being limiting, acrylic acid may be made from 3-HP via a dehydration reaction, which may be achieved by a number of commercial methodologies including via a distillation process, which may be part of the separation regime and which may include an acid and/or a metal ion as catalyst. More broadly, incorporated herein for its teachings of conversion of 3-HP, and other β-hydroxy carbonyl compounds, to acrylic acid and other related downstream compounds, is U.S. Patent Publication No. 2007/0219390 A1, published Sep. 20, 2007, now abandoned. This publication lists numerous catalysts and provides examples of conversions, which are specifically incorporated herein.

For example, including as exemplified from such incorporated references, 3-HP may be dehydrated to acrylic acid via various specific methods, each often involving one or more dehydration catalysts. One catalyst of particular apparent value is titanium, such as in the form of titanium oxide, TiO₂. A titanium dioxide catalyst may be provided in a dehydration system that distills an aqueous solution comprising 3-HP, wherein the 3-HP dehydrates, such as upon volatilization, converting to acrylic acid, and the acrylic acid is collected by condensation from the vapor phase.

As but one specific method, an aqueous solution of 3-HP is passed through a reactor column packed with a titanium oxide catalyst maintained at a temperature between 170 and 190° C. and at ambient atmospheric pressure. Vapors leaving the reactor column are passed over a low temperature condenser, where acrylic acid is collected. The low temperature condenser may be cooled to 30° C. or less, 20° C. or less, 2° C. or less, or at any suitable temperature for efficient condensation based on the flow rate and design of the system. Also, the reactor column temperatures may be lower, for instance when operating at a pressure lower than ambient atmospheric pressure. It is noted that Example 1 of U.S. Patent Publication No. 2007/0219390, published Sep. 20, 2007, now abandoned, provides specific parameters that employs the approach of this method. As noted, this publication is incorporated by reference for this teaching and also for its listing of catalysts that may be used in a 3-HP to acrylic acid dehydration reaction.

Crystallization of the acrylic acid obtained by dehydration of 3-HP may be used as one of the final separation/purification steps. Various approaches to crystallization are known in the art, including crystallization of esters.

As noted above, in some embodiments, a salt of 3-HP is converted to acrylic acid or an ester or salt thereof. For example, U.S. Pat. No. 7,186,856 (Meng et al.) teaches a process for producing acrylic acid from the ammonium salt of 3-HP, which involves a first step of heating the ammonium salt of 3-HP in the presence of an organic amine or solvent that is immiscible with water, to form a two-phase solution and split the 3-HP salt into its respective ionic constituents under conditions which transfer 3-HP from the aqueous phase to the organic phase of the solution, leaving ammonia and ammonium cations in the aqueous phase. The organic phase is then back-extracted to separate the 3-HP, followed by a second step of heating the 3-HP-containing solution in the presence of a dehydration catalyst to produce acrylic acid. U.S. Pat. No. 7,186,856 is incorporated by reference for its methods for producing acrylic acid from salts of 3-HP. Various alternatives to the particular approach disclosed in this patent may be developed for suitable extraction and conversion processes.

Methyl acrylate may be made from 3-HP via dehydration and esterification, the latter to add a methyl group (such as using methanol), acrylamide may be made from 3-HP via dehydration and amidation reactions, acrylonitrile may be made via a dehydration reaction and forming a nitrile moiety, propriolactone may be made from 3-HP via a ring-forming internal esterification reaction (eliminating a water molecule), ethyl-3-HP may be made from 3-HP via esterification with ethanol, malonic acid may be made from 3-HP via an oxidation reaction, and 1,3-propanediol may be made from 3-HP via a reduction reaction.

Malonic acid may be produced from oxidation of 3-HP as produced herein. U.S. Pat. No. 5,817,870 (Haas et al.) discloses catalytic oxidation of 3-HP by a precious metal selected from Ru, Rh, Pd, Os, Ir or Pt. These can be pure metal catalysts or supported catalysts. The catalytic oxidation can be carried out using a suspension catalyst in a suspension reactor or using a fixed-bed catalyst in a fixed-bed reactor. If the catalyst, preferably a supported catalyst, is disposed in a fixed-bed reactor, the latter can be operated in a trickle-bed procedure as well as also in a liquid-phase procedure. In the trickle-bed procedure the aqueous phase comprising the 3-HP starting material, as well as the oxidation products of the same and means for the adjustment of pH, and oxygen or an oxygen-containing gas can be conducted in parallel flow or counter-flow. In the liquid-phase procedure the liquid phase and the gas phase are conveniently conducted in parallel flow.

In order to achieve a sufficiently short reaction time, the conversion is carried out at a pH equal or greater than 6, preferably at least 7, and in particular between 7.5 and 9. According to a preferred embodiment, during the oxidation reaction the pH is kept constant, preferably at a pH in the range between 7.5 and 9, by adding a base, such as an alkaline or alkaline earth hydroxide solution. The oxidation is usefully carried out at a temperature of at least 10° C. and maximally 70° C. The flow of oxygen is not limited. In the suspension method it is important that the liquid and the gaseous phase are brought into contact by stirring vigorously. Malonic acid can be obtained in nearly quantitative yields. U.S. Pat. No. 5,817,870 is incorporated by reference herein for its methods to oxidize 3-HP to malonic acid.

1,3-Propanediol may be produced from hydrogenation of 3-HP as produced herein. U.S. Patent Publication No. 2005/0283029 (Meng et al.) is incorporated by reference herein for its methods to hydrogenation of 3-HP, or esters of the acid or mixtures, in the presence of a specific catalyst, in a liquid phase, to prepare 1,3-propanediol. Possible catalysts include ruthenium metal, or compounds of ruthenium, supported or unsupported, alone or in combination with at least one or more additional metal(s) selected from molybdenum, tungsten, titanium, zirconium, niobium, vanadium or chromium. The ruthenium metal or compound thereof, and/or the additional metal(s), or compound thereof, may be utilized in supported or unsupported form. If utilized in supported form, the method of preparing the supported catalyst is not critical and can be any technique such as impregnation of the support or deposition on the support. Any suitable support may be utilized. Supports that may be used include, but are not limited to, alumina, titania, silica, zirconia, carbons, carbon blacks, graphites, silicates, zeolites, aluminosilicate zeolites, aluminosilicate clays, and the like.

The hydrogenation process may be carried out in liquid phase. The liquid phase includes water, organic solvents that are not hydrogenatable, such as any aliphatic or aromatic hydrocarbon, alcohols, ethers, toluene, decalin, dioxane, diglyme, n-heptane, hexane, xylene, benzene, tetrahydrofuran, cyclohexane, methylcyclohexane, and the like, and mixtures of water and organic solvent(s). The hydrogenation process may be carried out batch wise, semi-continuously, or continuously. The hydrogenation process may be carried out in any suitable apparatus. Exemplary of such apparatus are stirred tank reactors, trickle-bed reactors, high pressure hydrogenation reactors, and the like.

The hydrogenation process is generally carried out at a temperature ranging from about 20 to about 250° C., more particularly from about 100 to about 200° C. Further, the hydrogenation process is generally carried out in a pressure range of from about 20 psi to about 4000 psi. The hydrogen containing gas utilized in the hydrogenation process is, optionally, commercially pure hydrogen. The hydrogen containing gas is usable if nitrogen, gaseous hydrocarbons, or oxides of carbon, and similar materials, are present in the hydrogen containing gas. For example, hydrogen from synthesis gas (hydrogen and carbon monoxide) may be employed, such synthesis gas potentially further including carbon dioxide, water, and various impurities.

As is known in the art, it is also possible to convert 3-HP to 1,3-propanediol using biological methods. For example, 1,3-propanediol can be created from either 3-HP-CoA or 3-HP via the use of polypeptides having enzymatic activity. These polypeptides can be used either in vitro or in vivo. When converting 3-HP-CoA to 1,3-propanediol, polypeptides having oxidoreductase activity or reductase activity (e.g., enzymes from the 1.1.1.-class of enzymes) can be used. Alternatively, when creating 1,3-propanediol from 3-HP, a combination of a polypeptide having aldyhyde dehydrogenase activity (e.g., an enzyme from the 1.1.1.34 class) and a polypeptide having alcohol dehydrogenase activity (e.g., an enzyme from the 1.1.1.32 class) can be used.

Another downstream production of 3-HP, acrylonitrile, may be converted from acrylic acid by various organic syntheses, including by not limited to the Sohio acrylonitrile process, a single-step method of production known in the chemical manufacturing industry

Also, addition reactions may yield acrylic acid or acrylate derivatives having alkyl or aryl groups at the carbonyl hydroxyl group. Such additions may be catalyzed chemically, such as by hydrogen, hydrogen halides, hydrogen cyanide, or Michael additions under alkaline conditions optionally in the presence of basic catalysts. Alcohols, phenols, hydrogen sulfide, and thiols are known to add under basic conditions. Aromatic amines or amides, and aromatic hydrocarbons, may be added under acidic conditions. These and other reactions are described in Ulmann's Encyclopedia of Industrial Chemistry, Acrylic Acid and Derivatives, WileyVCH Verlag GmbH, Wienham (2005), incorporated by reference for its teachings of conversion reactions for acrylic acid and its derivatives.

Acrylic acid obtained from 3-HP made by the present invention may be further converted to various chemicals, including polymers, which are also considered downstream products in some embodiments. Acrylic acid esters may be formed from acrylic acid (or directly from 3-HP) such as by condensation esterification reactions with an alcohol, releasing water. This chemistry described in Monomeric Acrylic Esters, E. H. Riddle, Reinhold, N.Y. (1954), incorporated by reference for its esterification teachings. Among esters that are formed are methyl acrylate, ethyl acrylate, n-butyl acrylate, hydroxypropyl acrylate, hydroxyethyl acrylate, isobutyl acrylate, and 2-ethylhexyl acrylate, and these and/or other acrylic acid and/or other acrylate esters may be combined, including with other compounds, to form various known acrylic acid-based polymers. Although acrylamide is produced in chemical syntheses by hydration of acrylonitrile, herein a conversion may convert acrylic acid to acrylamide by amidation.

Acrylic acid obtained from 3-HP made by the present invention may be further converted to various chemicals, including polymers, which are also considered downstream products in some embodiments. Acrylic acid esters may be formed from acrylic acid (or directly from 3-HP) such as by condensation esterification reactions with an alcohol, releasing water. This chemistry is described in Monomeric Acrylic Esters, E. H. Riddle, Reinhold, N.Y. (1954), incorporated by reference for its esterification teachings. Among esters that are formed are methyl acrylate, ethyl acrylate, n-butyl acrylate, hydroxypropyl acrylate, hydroxyethyl acrylate, isobutyl acrylate, and 2-ethylhexyl acrylate, and these and/or other acrylic acid and/or other acrylate esters may be combined, including with other compounds, to form various known acrylic acid-based polymers. Although acrylamide is produced in chemical syntheses by hydration of acrylonitrile, herein a conversion may convert acrylic acid to acrylamide by amidation.

Direct esterification of acrylic acid can take place by esterification methods known to the person skilled in the art, by contacting the acrylic acid obtained from 3-HP dehydration with one or more alcohols, such as methanol, ethanol, 1-propanol, 2-propanol, n-butanol, tert-butanol or isobutanol, and heating to a temperature of at least 50, 75, 100, 125, or 150° C. The water formed during esterification may be removed from the reaction mixture, such as by azeotropic distillation through the addition of suitable separation aids, or by another means of separation. Conversions up to 95%, or more, may be realized, as is known in the art.

Several suitable esterification catalysts are commercially available, such as from Dow Chemical (Midland, Mich. US). For example, Amberlyst™ 131Wet Monodisperse gel catalyst confers enhanced hydraulic and reactivity properties and is suitable for fixed bed reactors. Amberlyst™ 39Wet is a macroreticular catalyst suitable particularly for stirred and slurry loop reactors. Amberlyst™ 46 is a macroporous catalyst producing less ether byproducts than conventional catalyst (as described in U.S. Pat. No. 5,426,199 to Rohm and Haas, which patent is incorporated by reference for its teachings of esterification catalyst compositions and selection considerations).

Acrylic acid, and any of its esters, may be further converted into various polymers. Polymerization may proceed by any of heat, light, other radiation of sufficient energy, and free radical generating compounds, such as azo compounds or peroxides, to produce a desired polymer of acrylic acid or acrylic acid esters. As one example, an aqueous acrylic acid solution's temperature raised to a temperature known to start polymerization (in part based on the initial acrylic acid concentration), and the reaction proceeds, the process frequently involving heat removal given the high exothermicity of the reaction. Many other methods of polymerization are known in the art. Some are described in Ulmann's Encyclopedia of Industrial Chemistry, Polyacrylamides and Poly(Acrylic Acids), WileyVCH Verlag GmbH, Wienham (2005), incorporated by reference for its teachings of polymerization reactions.

For example, the free-radical polymerization of acrylic acid takes place by polymerization methods known to the skilled worker and can be carried out either in an emulsion or suspension in aqueous solution or another solvent. Initiators, such as but not limited to organic peroxides, often are added to aid in the polymerization. Among the classes of organic peroxides that may be used as initiators are diacyls, peroxydicarbonates, monoperoxycarbonates, peroxyketals, peroxyesters, dialkyls, and hydroperoxides. Another class of initiators is azo initiators, which may be used for acrylate polyermization as well as co-polymerization with other monomers. U.S. Pat. Nos. 5,470,928; 5,510,307; 6,709,919; and 7,678,869 teach various approaches to polymerization using a number of initiators, including organic peroxides, azo compounds, and other chemical types, and are incorporated by reference for such teachings as applicable to the polymers described herein.

Accordingly, it is further possible for co-monomers, such as crosslinkers, to be present during the polymerization. The free-radical polymerization of the acrylic acid obtained from dehydration of 3-HP, as produced herein, in at least partly neutralized form and in the presence of crosslinkers is practiced in certain embodiments. This polymerization may result in hydrogels which can then be comminuted, ground and, where appropriate, surface-modified, by known techniques.

An important commercial use of polyacrylic acid is for superabsorbent polymers. This specification hereby incorporates by reference Modern Superabsorbent Polymer Technology, Buchholz and Graham (Editors), Wiley-VCH, 1997, in its entirety for its teachings regarding superabsorbent polymers components, manufacture, properties and uses. Superabsorbent polymers are primarily used as absorbents for water and aqueous solutions for diapers, adult incontinence products, feminine hygiene products, and similar consumer products. In such consumer products, superabsorbent materials can replace traditional absorbent materials such as cloth, cotton, paper wadding, and cellulose fiber. Superabsorbent polymers absorb, and retain under a slight mechanical pressure, up to 25 times or their weight in liquid. The swollen gel holds the liquid in a solid, rubbery state and prevents the liquid from leaking. Superabsorbent polymer particles can be surface-modified to produce a shell structure with the shell being more highly crosslinked. This technique improves the balance of absorption, absorption under load, and resistance to gel-blocking. It is recognized that superabsorbent polymers have uses in fields other than consumer products, including agriculture, horticulture, and medicine.

Superabsorbent polymers are prepared from acrylic acid (such as acrylic acid derived from 3-HP provided herein) and a crosslinker, by solution or suspension polymerization. Exemplary methods include U.S. Pat. Nos. 5,145,906; 5,350,799; 5,342,899; 4,857,610; 4,985,518; 4,708,997; 5,180,798; 4,666,983; 4,734,478; and 5,331,059, each incorporated by reference for their teachings relating to superabsorbent polymers.

Among consumer products, a diaper, a feminine hygiene product, and an adult incontinence product are made with superabsorbent polymer that itself is made substantially from acrylic acid converted from 3-HP made in accordance with the present invention.

Diapers and other personal hygiene products may be produced that incorporate superabsorbent polymer made from acrylic acid made from 3-HP which is bio-produced by the teachings of the present application. The following provides general guidance for making a diaper that incorporates such superabsorbent polymer. The superabsorbent polymer first is prepared into an absorbent pad that may be vacuum formed, and in which other materials, such as a fibrous material (e.g., wood pulp) are added. The absorbent pad then is assembled with sheet(s) of fabric, generally a nonwoven fabric (e.g., made from one or more of nylon, polyester, polyethylene, and polypropylene plastics) to form diapers.

More particularly, in one non-limiting process, above a conveyer belt multiple pressurized nozzles spray superabsorbent polymer particles (such as about 400 micron size or larger), fibrous material, and/or a combination of these onto the conveyer belt at designated spaces/intervals. The conveyor belt is perforated and under vacuum from below, so that the sprayed on materials are pulled toward the belt surface to form a flat pad. In various embodiments, fibrous material is applied first on the belt, followed by a mixture of fibrous material and the superabsorbent polymer particles, followed by fibrous material, so that the superabsorbent polymer is concentrated in the middle of the pad. A leveling roller may be used toward the end of the belt path to yield pads of uniform thickness. Each pad thereafter may be further processed, such as to cut it to a proper shape for the diaper, or the pad may be in the form of a long roll sufficient for multiple diapers. Thereafter, the pad is sandwiched between a top sheet and a bottom sheet of fabric (one generally being liquid pervious, the other liquid impervious), such as on a conveyor belt, and these are attached together such as by gluing, heating or ultrasonic welding, and cut into diaper-sized units (if not previously so cut). Additional features may be provided, such as elastic components, strips of tape, etc., for fit and ease of wearing by a person.

The ratio of the fibrous material to polymer particles is known to effect performance characteristics. In some embodiments, this ratio is between 75:25 and 90:10 (see U.S. Pat. No. 4,685,915, incorporated by reference for its teachings of diaper manufacture). Other disposable absorbent articles may be constructed in a similar fashion, such as for adult incontinence, feminine hygiene (sanitary napkins), tampons, etc. (see, for example, U.S. Pat. Nos. 5,009,653, 5,558,656, and 5,827,255 incorporated by reference for their teachings of sanitary napkin manufacture).

Low molecular-weight polyacrylic acid has uses for water treatment, flocculants, and thickeners for various applications including cosmetics and pharmaceutical preparations. For these applications, the polymer may be uncrosslinked or lightly crosslinked, depending on the specific application. The molecular weights are typically from about 200 to about 1,000,000 g/mol. Preparation of these low molecular-weight polyacrylic acid polymers is described in U.S. Pat. Nos. 3,904,685; 4,301,266; 2,798,053; and 5,093,472, each of which is incorporated by reference for its teachings relating to methods to produce these polymers.

Acrylic acid may be co-polymerized with one or more other monomers selected from acrylamide, 2-acrylamido-2-methylpropanesulfonic acid, N,N-dimethylacrylamide, N-isopropylacrylamide, methacrylic acid, and methacrylamide, to name a few. The relative reactivities of the monomers affect the microstructure and thus the physical properties of the polymer. Co-monomers may be derived from 3-HP, or otherwise provided, to produce co-polymers. Ulmann's Encyclopedia of Industrial Chemistry, Polyacrylamides and Poly(Acrylic Acids), WileyVCH Verlag GmbH, Wienham (2005), is incorporated by reference herein for its teachings of polymer and co-polymer processing.

Acrylic acid can in principle be copolymerized with almost any free-radically polymerizable monomers including styrene, butadiene, acrylonitrile, acrylic esters, maleic acid, maleic anhydride, vinyl chloride, acrylamide, itaconic acid, and so on. End-use applications typically dictate the co-polymer composition, which influences properties. Acrylic acid also may have a number of optional substitutions on it, and after such substitutions be used as a monomer for polymerization, or co-polymerization reactions. As a general rule, acrylic acid (or one of its co-polymerization monomers) may be substituted by any substituent that does not interfere with the polymerization process, such as alkyl, alkoxy, aryl, heteroaryl, benzyl, vinyl, allyl, hydroxy, epoxy, amide, ethers, esters, ketones, maleimides, succinimides, sulfoxides, glycidyl and silyl (see U.S. Pat. No. 7,678,869, incorporated by reference above, for further discussion). The following paragraphs provide a few non-limiting examples of copolymerization applications.

Paints that comprise polymers and copolymers of acrylic acid and its esters are in wide use as industrial and consumer products. Aspects of the technology for making such paints can be found in U.S. Pat. Nos. 3,687,885 and 3,891,591, incorporated by reference for its teachings of such paint manufacture. Generally, acrylic acid and its esters may form homopolymers or copolymers among themselves or with other monomers, such as amides, methacrylates, acrylonitrile, vinyl, styrene and butadiene. A desired mixture of homopolymers and/or copolymers, referred to in the paint industry as ‘vehicle’ (or ‘binder’) are added to an aqueous solution and agitated sufficiently to form an aqueous dispersion that includes sub-micrometer sized polymer particles. The paint cures by coalescence of these ‘vehicle’ particles as the water and any other solvent evaporate. Other additives to the aqueous dispersion may include pigment, filler (e.g., calcium carbonate, aluminum silicate), solvent (e.g., acetone, benzol, alcohols, etc., although these are not found in certain no VOC paints), thickener, and additional additives depending on the conditions, applications, intended surfaces, etc. In many paints, the weight percent of the vehicle portion may range from about nine to about 26 percent, but for other paints the weight percent may vary beyond this range.

Acrylic-based polymers are used for many coatings in addition to paints. For example, for paper coating latexes, acrylic acid is used from 0.1-5.0%, along with styrene and butadiene, to enhance binding to the paper and modify rheology, freeze-thaw stability and shear stability. In this context, U.S. Pat. Nos. 3,875,101 and 3,872,037 are incorporated by reference for their teachings regarding such latexes. Acrylate-based polymers also are used in many inks, particularly UV curable printing inks. For water treatment, acrylamide and/or hydroxy ethyl acrylate are commonly co-polymerized with acrylic acid to produce low molecular-weight linear polymers. In this context, U.S. Pat. Nos. 4,431,547 and 4,029,577 are incorporated by reference for their teachings of such polymers. Co-polymers of acrylic acid with maleic acid or itaconic acid are also produced for water-treatment applications, as described in U.S. Pat. No. 5,135,677, incorporated by reference for that teaching. Sodium acrylate (the sodium salt of glacial acrylic acid) can be co-polymerized with acrylamide (which may be derived from acrylic acid via amidation chemistry) to make an anionic co-polymer that is used as a flocculant in water treatment.

For thickening agents, a variety of co-monomers can be used, such as described in U.S. Pat. Nos. 4,268,641 and 3,915,921, incorporated by reference for description of these co-monomers. U.S. Pat. No. 5,135,677 describes a number of co-monomers that can be used with acrylic acid to produce water-soluble polymers, and is incorporated by reference for such description.

Also as noted, some conversions to downstream products may be made enzymatically. For example, 3-HP may be converted to 3-HP-CoA, which then may be converted into polymerized 3-HP with an enzyme having polyhydroxyacid synthase activity (EC 2.3.1.-). Also, 1,3-propanediol can be made using polypeptides having oxidoreductase activity or reductase activity (e.g., enzymes in the EC 1.1.1.-class of enzymes). Alternatively, when creating 1,3-propanediol from 3HP, a combination of (1) a polypeptide having aldehyde dehydrogenase activity (e.g., an enzyme from the 1.1.1.34 class) and (2) a polypeptide having alcohol dehydrogenase activity (e.g., an enzyme from the 1.1.1.32 class) can be used. Polypeptides having lipase activity may be used to form esters. Enzymatic reactions such as these may be conducted in vitro, such as using cell-free extracts, or in vivo.

Thus, various embodiments of the present invention, such as methods of making a chemical, include conversion steps to any such noted downstream products of microbially produced 3-HP, including but not limited to those chemicals described herein and in the incorporated references (the latter for jurisdictions allowing this). For example, one embodiment is making 3-HP molecules by the teachings herein and further converting the 3-HP molecules to polymerized-3-HP (poly-3-HP) or acrylic acid, and such as from acrylic acid then producing from the 3-HP molecules any one of polyacrylic acid (polymerized acrylic acid, in various forms), methyl acrylate, acrylamide, acrylonitrile, propiolactone, ethyl 3-HP, malonic acid, 1,3-propanediol, ethyl acrylate, n-butyl acrylate, hydroxypropyl acrylate, hydroxyethyl acrylate, isobutyl acrylate, 2-ethylhexyl acrylate, and acrylic acid or an acrylic acid ester to which an alkyl or aryl addition is made, and/or to which halogens, aromatic amines or amides, and aromatic hydrocarbons are added.

Also as noted, some conversions to downstream products may be made enzymatically. For example, 3-HP may be converted to 3-HP-CoA, which then may be converted into polymerized 3-HP with an enzyme having polyhydroxyacid synthase activity (EC 2.3.1.-). Also, 1,3-propanediol can be made using polypeptides having oxidoreductase activity or reductase activity (e.g., enzymes in the EC 1.1.1.-class of enzymes). Alternatively, when creating 1,3-propanediol from 3HP, a combination of (1) a polypeptide having aldehyde dehydrogenase activity (e.g., an enzyme from the 1.1.1.34 class) and (2) a polypeptide having alcohol dehydrogenase activity (e.g., an enzyme from the 1.1.1.32 class) can be used. Polypeptides having lipase activity may be used to form esters. Enzymatic reactions such as these may be conducted in vitro, such as using cell-free extracts, or in vivo.

Thus, various embodiments of the present invention, such as methods of making a chemical, include conversion steps to any such noted downstream products of microbially produced 3-HP, including but not limited to those chemicals described herein and in the incorporated references (the latter for jurisdictions allowing this). For example, one embodiment is making 3-HP molecules by the teachings herein and further converting the 3-HP molecules to polymerized-3-HP (poly-3-HP) or acrylic acid, and such as from acrylic acid then producing from the 3-HP molecules any one of polyacrylic acid (polymerized acrylic acid, in various forms), methyl acrylate, acrylamide, acrylonitrile, propiolactone, ethyl 3-HP, malonic acid, 1,3-propanediol, ethyl acrylate, n-butyl acrylate, hydroxypropyl acrylate, hydroxyethyl acrylate, isobutyl acrylate, 2-ethylhexyl acrylate, and acrylic acid or an acrylic acid ester to which an alkyl or aryl addition is made, and/or to which halogens, aromatic amines or amides, and aromatic hydrocarbons are added.

Reactions that form downstream compounds such as acrylates or acrylamides can be conducted in conjunction with use of suitable stabilizing agents or inhibiting agents reducing likelihood of polymer formation. See, for example, U.S. Patent Publication No. 2007/0219390 A1. Stabilizing agents and/or inhibiting agents include, but are not limited to, e.g., phenolic compounds (e.g., dimethoxyphenol (DMP) or alkylated phenolic compounds such as di-tert-butyl phenol), quinones (e.g., t-butyl hydroquinone or the monomethyl ether of hydroquinone (MEHQ)), and/or metallic copper or copper salts (e.g., copper sulfate, copper chloride, or copper acetate). Inhibitors and/or stabilizers can be used individually or in combinations as will be known by those of skill in the art. Also, in various embodiments, the one or more downstream compounds is/are recovered at a molar yield of up to about 100 percent, or a molar yield in the range from about 70 percent to about 90 percent, or a molar yield in the range from about 80 percent to about 100 percent, or a molar yield in the range from about 90 percent to about 100 percent. Such yields may be the result of single-pass (batch or continuous) or iterative separation and purification steps in a particular process.

Acrylic acid and other downstream products are useful as commodities in manufacturing, such as in the manufacture of consumer goods, including diapers, textiles, carpets, paints, and adhesives, as well as lesser known consumer goods, such as cross-linked polyacrylamide-containing products marketed for soil moisture retention for household plants and gardens.

Nucleic acid and amino acid sequences are provided herein and their use in compositions, methods and systems as described herein are within the scope of the present invention. Also, where certain gene or enzyme names, or EC numbers for respective reactions are provided herein, it is understood that the respective nucleic acid and amino acid sequences are within the scope of the present invention. When a gene or enzyme name or other identifier is provided, one skilled in the art can readily obtain a corresponding sequence, such as from various public databases, including but not limited to those available at http://www.ncbi.nlm nih.gov/sites/entrez/?db=gene, http://www.ncbi.nlm.nih.gov/sites/entrez?db=Protein&itool=toolbar, www.ecocyc.org, and www.metacyc.org. Also, sequences are not provided for pathways existing in a native host cell, although improvements thereto may be made in various embodiments and/or in conjunction with embodiments of the present invention.

Also, the scope of the present invention is not meant to be limited to the exact sequences provided herein. It is appreciated that a range of modifications to nucleic acid and to amino acid sequences may be made and still provide a desired functionality, such as a desired enzymatic activity and specificity. The following discussion is provided describe ranges of variation that may be practiced and still remain within the scope of the present invention.

It has long been recognized in the art that some amino acids in amino acid sequences can be varied without significant effect on the structure or function of proteins. Variants included can constitute deletions, insertions, inversions, repeats, and type substitutions so long as the indicated enzyme activity is not significantly adversely affected.

Examples of properties that provide the bases for conservative and other amino acid substitutions are exemplified in Table 4. Accordingly, one skilled in the art may make numerous substitutions to obtain an amino acid sequence variant that exhibits a desired functionality. BLASTP, CLUSTALP, and other alignment and comparison tools may be used to assess highly conserved regions, to which fewer substitutions may be made (unless directed to alter activity to a selected level, which may require multiple substitutions). More substitutions may be made in regions recognized or believed to not be involved with an active site or other binding or structural motif. In accordance with Table 3, for example, substitutions may be made of one polar uncharged (PU) amino acid for a polar uncharged amino acid of a listed sequence, optionally considering size/molecular weight (i.e., substituting a serine for a threonine). Guidance concerning which amino acid changes are likely to be phenotypically silent can be found, inter alia, in Bowie, J. U., et Al., “Deciphering the Message in Protein Sequences: Tolerance to Amino Acid Substitutions,” Science 247:1306-1310 (1990). This reference is incorporated by reference for such teachings, which are, however, also generally known to those skilled in the art. Recognized conservative amino acid substitutions comprise (substitutable amino acids following each colon of a set): ala:ser; arg:lys; asn:gln or his; asp:glu; cys:ser; gln:asn; glu:asp; gly:pro; his:asn or gln; ile:leu or val; leu:ile or val; lys: arg or gln or glu; met:leu or ile; phe:met or leu or tyr; ser:thr; thr:ser; trp:tyr; tyr:trp or phe; val:ile or leu.

It is noted that codon preferences and codon usage tables for a particular species can be used to engineer isolated nucleic acid molecules that take advantage of the codon usage preferences of that particular species. For example, the isolated nucleic acid provided herein can be designed to have codons that are preferentially used by a particular organism of interest. Numerous software and sequencing services are available for such codon-optimizing of sequences.

The invention provides polypeptides that contain the entire amino acid sequence of an amino acid sequence listed or otherwise disclosed herein. In addition, the invention provides polypeptides that contain a portion of an amino acid sequence listed or otherwise disclosed herein. For example, the invention provides polypeptides that contain a 15 amino acid sequence identical to any 15 amino acid sequence of an amino acid sequence listed or otherwise disclosed herein including, without limitation, the sequence starting at amino acid residue number 1 and ending at amino acid residue number 15, the sequence starting at amino acid residue number 2 and ending at amino acid residue number 16, the sequence starting at amino acid residue number 3 and ending at amino acid residue number 17, and so forth. It will be appreciated that the invention also provides polypeptides that contain an amino acid sequence that is greater than 15 amino acid residues (e.g., 16, 17, 18, 19, 20, 21, 22, 23, 24, 25, 26, 27, 28, 29, 30 or more amino acid residues) in length and identical to any portion of an amino acid sequence listed or otherwise disclosed herein. For example, the invention provides polypeptides that contain a 25 amino acid sequence identical to any 25 amino acid sequence of an amino acid sequence listed or otherwise disclosed herein including, without limitation, the sequence starting at amino acid residue number 1 and ending at amino acid residue number 25, the sequence starting at amino acid residue number 2 and ending at amino acid residue number 26, the sequence starting at amino acid residue number 3 and ending at amino acid residue number 27, and so forth. Additional examples include, without limitation, polypeptides that contain an amino acid sequence that is 50 or more amino acid residues (e.g., 100, 150, 200, 250, 300 or more amino acid residues) in length and identical to any portion of an amino acid sequence listed or otherwise disclosed herein. Further, it is appreciated that, per above, a 15 nucleotide sequence will provide a 5 amino acid sequence, so that the latter, and higher-length amino acid sequences, may be defined by the above-described nucleotide sequence lengths having identity with a sequence provided herein.

In various embodiments polypeptides obtained by the expression of the polynucleotide molecules of the present invention may have at least approximately 50%, 60%, 70%, 80%, 90%, 95%, 96%, 97%, 98%, 99% or 100% identity to one or more amino acid sequences encoded by the genes and/or nucleic acid sequences described herein for the biosynthesis reactions and pathways. A truncated respective polypeptide has at least about 90% of the full length of a polypeptide encoded by a nucleic acid sequence encoding the respective native enzyme, and more particularly at least 95% of the full length of a polypeptide encoded by a nucleic acid sequence encoding the respective native enzyme. By a polypeptide having an amino acid sequence at least, for example, 95% “identical” to a reference amino acid sequence of a polypeptide is intended that the amino acid sequence of the claimed polypeptide is identical to the reference sequence except that the claimed polypeptide sequence can include up to five amino acid alterations per each 100 amino acids of the reference amino acid of the polypeptide. In other words, to obtain a polypeptide having an amino acid sequence at least 95% identical to a reference amino acid sequence, up to 5% of the amino acid residues in the reference sequence can be deleted or substituted with another amino acid, or a number of amino acids up to 5% of the total amino acid residues in the reference sequence can be inserted into the reference sequence. These alterations of the reference sequence can occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 50%, 60%, 70%, 80%, 85%, 90%, 92%, 95%, 96%, 97%, 98% or 99% identical to any reference amino acid sequence of any polypeptide described herein (which may correspond with a particular nucleic acid sequence described herein), such particular polypeptide sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set such that the percentage of identity is calculated over the full length of the reference amino acid sequence and that gaps in identity of up to 5% of the total number of amino acid residues in the reference sequence are allowed.

For example, in a specific embodiment the identity between a reference sequence (query sequence, i.e., a sequence of the present invention) and a subject sequence, also referred to as a global sequence alignment, may be determined using the FASTDB computer program based on the algorithm of Brutlag et al. (Comp. App. Biosci. 6:237-245 (1990)). Particular parameters for a particular embodiment in which identity is narrowly construed, used in a FASTDB amino acid alignment, are: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. According to this embodiment, if the subject sequence is shorter than the query sequence due to N- or C-terminal deletions, not because of internal deletions, a manual correction is made to the results to take into consideration the fact that the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are lateral to the N- and C-terminal of the subject sequence, which are not matched (i.e., aligned) with a corresponding subject residue, as a percent of the total bases of the query sequence. A determination of whether a residue is matched (i.e., aligned) is determined by results of the FASTDB sequence alignment. This percentage is then subtracted from the percent identity, calculated by the above FASTDB program using the specified parameters, to arrive at a final percent identity score. This final percent identity score is what is used for the purposes of this embodiment. Only residues to the N- and C-termini of the subject sequence, which are not matched (i.e., aligned) with the query sequence, are considered for the purposes of manually adjusting the percent identity score. That is, only query residue positions outside the farthest N- and C-terminal residues of the subject sequence are considered for this manual correction. For example, a 90 amino acid residue subject sequence is aligned with a 100 residue query sequence to determine percent identity. The deletion occurs at the N-terminus of the subject sequence and therefore, the FASTDB alignment does not show a matching (i.e., alignment) of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched (i.e., aligned) with the query. In this case the percent identity calculated by FASTDB is not manually corrected. Once again, only residue positions outside the N- and C-terminal ends of the subject sequence, as displayed in the FASTDB alignment, which are not matched (i.e., aligned) with the query sequence are manually corrected for.

Also as used herein, the term “homology” refers to the optimal alignment of sequences (either nucleotides or amino acids), which may be conducted by computerized implementations of algorithms. “Homology”, with regard to polynucleotides, for example, may be determined by analysis with BLASTN version 2.0 using the default parameters. “Homology” with respect to polypeptides (i.e., amino acids), may be determined using a program, such as BLASTP version 2.2.2 with the default parameters, which aligns the polypeptides or fragments being compared and determines the extent of amino acid identity or similarity between them. It will be appreciated that amino acid homologues includes conservative substitutions, i.e. those that substitute a given amino acid in a polypeptide by another amino acid of similar characteristics. Typically seen as conservative substitutions are the following replacements: replacements of an aliphatic amino acid such as Ala, Val, Leu and Ile with another aliphatic amino acid; replacement of a Ser with a Thr or vice versa; replacement of an acidic residue such as Asp or Glu with another acidic residue; replacement of a residue bearing an amide group, such as Asn or Gln, with another residue bearing an amide group; exchange of a basic residue such as Lys or Arg with another basic residue; and replacement of an aromatic residue such as Phe or Tyr with another aromatic residue. A polypeptide sequence (i.e., amino acid sequence) or a polynucleotide sequence comprising at least 50% homology to another amino acid sequence or another nucleotide sequence respectively has a homology of 50% or greater than 50%, e.g., 60%, 70%, 80%, 90% or 100%.

The above descriptions and methods for sequence identity and homology are intended to be exemplary and it is recognized that these concepts are well-understood in the art.

Further, it is appreciated that nucleic acid sequences may be varied and still encode an enzyme or other polypeptide exhibiting a desired functionality, and such variations are within the scope of the present invention, as are those and other sequences when directed to production of intermediate products (en route to 3-HP) and other products of commercial value other than 3-HP, all of which may be collectively referred to as “products.” Nucleic acid sequences that encode polypeptides that provide the indicated functions for increased 3-HP production are considered within the scope of the present invention. These may be further defined by the stringency of hybridization, described below, but this is not meant to be limiting when a function of an encoded polypeptide matches a specified biosynthesis pathway enzyme activity.

Further to nucleic acid sequences, “hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid” or “duplex.” “Hybridization conditions” will typically include salt concentrations of less than about 1M, more usually less than about 500 mM and less than about 200 mM. Hybridization temperatures can be as low as 5° C., but are typically greater than 22° C., more typically greater than about 30° C., and often are in excess of about 37° C. Hybridizations are usually performed under stringent conditions, i.e. conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the Tm for the specific sequence at a defined ionic strength and pH. Exemplary stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see for example, Sambrook and Russell and Anderson “Nucleic Acid Hybridization” 1st Ed., BIOS Scientific Publishers Limited (1999), which is hereby incorporated by reference for hybridization protocols. “Hybridizing specifically to” or “specifically hybridizing to” or like expressions refer to the binding, duplexing, or hybridizing of a molecule substantially to or only to a particular nucleotide sequence or sequences under stringent conditions when that sequence is present in a complex mixture (e.g., total cellular) DNA or RNA.

Accordingly, In yet other embodiments, an isolated nucleic acid molecule of the invention, or a microorganism of the invention, comprises a nucleic acid molecule which is a complement of one of the nucleotide sequences shown herein, such as in Table 1, or a portion thereof. As used herein, the term “complementary” refers to a nucleotide sequence that can hybridize to one of the nucleotide sequences listed in Table 1, the sequences provided in the sequence listing herein, thereby forming a stable duplex.

In one aspect of the invention the identity values in the preceding paragraphs are determined using the parameter set described above for the FASTDB software program. It is recognized that identity may be determined alternatively with other recognized parameter sets, and that different software programs (e.g., Bestfit vs. BLASTp). Thus, identity can be determined in various ways. Further, for all specifically recited sequences herein it is understood that conservatively modified variants thereof are intended to be included within the invention.

Thus, polynucleotide (nucleic acid) sequences and polypeptide (e.g., enzyme) sequences of the present invention may be grouped, or characterized, with reference to percent identity, percent homology, and/or degree of hybridization with, a specified sequence. Further, those skilled in the art will understand that the genetic modifications described herein, with reference to E. coli genes and their respective enzymatic activities, and for certain genes of other species, are not meant to be limiting. Given the complete genome sequencing of a large and increasing number of microorganism species, and the level of skill in the art, one skilled in the art will be able to apply the present teachings and disclosures to numerous other microorganisms of interest for increased production of 3-HP and other products.

Further to the determination of homologous genes in a selected microorganism species, this may be determined as follows. Using as a starting point a gene disclosed herein, one may conduct a homology search and analysis to obtain a listing of potentially homologous sequences for the selected microorganism species. For this homology approach a local blast (www.ncbi.nlm.nih.gov/Tools/) (blastp) comparison using the E. coli protein encoded by the selected gene is performed using different thresholds and comparing to one or more selected species (www.ncbi.nlm.nih.gov/genomes/lproks.cgi). A suitable E-value is chosen at least in part based on the number of results and the desired ‘tightness’ of the homology, considering the number of later evaluations to identify useful genes. Genes so identified may be evaluated in accordance with the teachings of the present invention. Such gene may encode an enzyme wherein that enzyme's amino acid sequence is within a 50, 60, 70, 80, 90, or 95 percent homology of the selected gene. It is noted that such identified and evaluated nucleic acid and amino acid sequences may also be selected, at least in part, by correspondence with the size of the selected gene.

Thus, using such approaches based on identifying sequences that have a specified homology to sequences disclosed herein (“reference sequences”), nucleic acid and amino acid sequences are identified, and may be evaluated and used in embodiments of the invention, wherein the latter nucleic acid and amino acid sequences fall within a specified percentage of sequence identity.

Also, variants or sequences having substantial identity or homology with the polynucleotides encoding enzymes described herein, and their functional equivalents in other species, may be assessed, and assuming a suitable specific functionality is determined (such as by evaluation of enzymatic activity), utilized in the practice and various embodiments of the present invention. Such sequences can be referred to as variants or modified sequences. That is, a polynucleotide sequence may be modified yet still retain the ability to encode a polypeptide exhibiting a desired enzymatic activity. Such variants or modified sequences are thus equivalents. Generally, the variant or modified sequence may comprise at least about 40 to 60 percent, or about 60 to 80 percent, or about 80 to 90 percent, or about 90 to 95 percent, or over 95 percent, sequence identity with the reference sequence (that sequence used to start the analysis).

Similarly, it is appreciated that the encoded amino acid sequence of the polypeptide exhibiting the enzymatic activity may vary and still retain the desired functionality. This may also be quantified by sequence identity, a term known to and applied by those skilled in the art.

In some embodiments, the invention contemplates a genetically modified (e.g., recombinant) microorganism comprising a heterologous nucleic acid sequence that encodes a polypeptide that is an identified enzymatic functional variant of any of the enzymes of the production pathway(s) disclosed herein, wherein the polypeptide has enzymatic activity and specificity effective to perform the enzymatic reaction of the respective production pathway enzyme, so that the recombinant microorganism exhibits greater 3-HP production than an appropriate control microorganism lacking such nucleic acid sequence. This also applies to other products described herein. Relevant methods of the invention also are intended to be directed to identifying variants that exhibit a desired enzymatic functionality, and the nucleic acid sequences that encode them.

In accordance with the teachings herein, including the examples, microorganisms are modified to provide increased production of desired organic chemical molecules, such as 3-HP, from the carbon sources carbon dioxide and/or carbon monoxide (which in some embodiments may also comprise more complex carbon sources, such as sugars). In making such modified microorganisms, iterative modifications may be made and evaluated, leading to cells having improved characteristics for such production. The modifications may include additions as well as deletions of genetic material.

Also, in various embodiments an oxygen-tolerant CO dehydrogenase complex may be provided for conversion of carbon monoxide to hydrogen in accordance with the water shift reaction (CO+H₂O->CO₂+H₂). Specific oxygen-tolerant genes that may be employed are known, e.g., see “The structural genes encoding CO dehydrogenase subunits (cox L, M and S) in Pseudomonas carboxydovorans OM5 reside on plasmid pHCG3 and are, with the exception of Streptomyces thermoautotrophicus, conserved in carboxydotrophic bacteria,” Iris Hugendieck and Ortwin Meyer (Archives of Microbiology, Volume 157, Number 3, 301-304, DOI: 10.1007/BF00245166. The C. carboxidovorans protein sequences for CoxL, CoxM, and CoxS are provided as SEQ ID NOs. 034, 035 and 036 (CAA57829.1 GI:809566, CAA57827.1 GI:809564, and CAA57828.1 GI:809565, respectively.) These additions may be combined with various other embodiments in any combination.

Also, and more generally, in accordance with disclosures, discussions, examples and embodiments herein, there may be employed conventional molecular biology, cellular biology, microbiology, and recombinant DNA techniques within the skill of the art. Such techniques are explained fully in the literature. (See, e.g., Sambrook and Russell, “Molecular Cloning: A Laboratory Manual,” Third Edition 2001 (volumes 1-3), Cold Spring Harbor Laboratory Press, Cold Spring Harbor, N.Y.; Animal Cell Culture, R. I. Freshney, ed., 1986). These published resources are incorporated by reference herein for their respective teachings of standard laboratory methods found therein. Such incorporation, at a minimum, is for the specific teaching and/or other purpose that may be noted when citing the reference herein. If a specific teaching and/or other purpose is not so noted, then the published resource is specifically incorporated for the teaching(s) indicated by one or more of the title, abstract, and/or summary of the reference. If no such specifically identified teaching and/or other purpose may be so relevant, then the published resource is incorporated in order to more fully describe the state of the art to which the present invention pertains, and/or to provide such teachings as are generally known to those skilled in the art, as may be applicable. However, it is specifically stated that a citation of a published resource herein shall not be construed as an admission that such is prior art to the present invention. Also, in the event that one or more of the incorporated published resources differs from or contradicts this application, including but not limited to defined terms, term usage, described techniques, or the like, this application controls.

While various embodiments of the present invention have been shown and described herein, it is emphasized that such embodiments are provided by way of example only. Numerous variations, changes and substitutions may be made without departing from the invention herein in its various embodiments. Specifically, and for whatever reason, for any grouping of compounds, nucleic acid sequences, polypeptides including specific proteins including functional enzymes, metabolic pathway enzymes or intermediates, elements, or other compositions, or concentrations stated or otherwise presented herein in a list, table, or other grouping (such as metabolic pathway enzymes shown in a figure), unless clearly stated otherwise, it is intended that each such grouping provides the basis for and serves to identify various subset embodiments, the subset embodiments in their broadest scope comprising every subset of such grouping by exclusion of one or more members (or subsets) of the respective stated grouping. Moreover, when any range is described herein, unless clearly stated otherwise, that range includes all values therein and all sub-ranges therein. Accordingly, it is intended that the invention be limited only by the spirit and scope of appended claims, and of later claims, and of either such claims as they may be amended during prosecution of this or a later application claiming priority hereto.

In various embodiments, production of 3-HP, or alternatively one of its downstream products such as described herein, may reach at least 1, at least 2, at least 5, at least 10, at least 20, at least 30, at least 40, and at least 50 g/liter titer, such as by using one of the methods disclosed herein.

### EXAMPLE(S)

Unless otherwise indicated, the following are examples planned to be conducted or actually conducted in Boulder, Colo., USA. Unless indicated otherwise, temperature is in degrees Celsius and pressure is at or near atmospheric pressure at approximately 5340 feet (1628 meters) above sea level. It is noted that work done at external analytical and synthetic facilities is not conducted at or near atmospheric pressure at approximately 5340 feet (1628 meters) above sea level. All reagents, unless otherwise indicated, are obtained commercially. Species and other phylogenic identifications are according to the classification known to a person skilled in the art of microbiology.

These examples are meant to be broadly exemplary and not limiting in any way. This applies to the examples regarding separation and purification of 3-HP, and conversions of 3-HP to downstream compounds, since there are numerous possible approaches to such steps and conversions, including those disclosed in references recited and incorporated herein.

The meaning of abbreviations is as follows: “C” means Celsius or degrees Celsius, as is clear from its usage, “s” means second(s), “min” means minute(s), “h,” “hr,” or “hrs” means hour(s), “psi” means pounds per square inch, “nm” means nanometers, “d” means day(s), “μL” or “uL” or “ul” means microliter(s), “mL” means milliliter(s), “L” means liter(s), “mm” means millimeter(s), “nm” means nanometers, “mM” means millimolar, “μM” or “uM” means micromolar, “M” means molar, “mmol” means millimole(s), “μmol” or “uMol” means micromole(s)“, “g” means gram(s), “μg” or “ug” means microgram(s) and “ng” means nanogram(s), “PCR” means polymerase chain reaction, “OD” means optical density, “OD₆₀₀” means the optical density measured at a photon wavelength of 600 nm, “kDa” means kilodaltons, “g” means the gravitation constant, “bp” means base pair(s), “kbp” means kilobase pair(s), “% w/v” means weight/volume percent, % v/v″ means volume/volume percent, “IPTG” means isopropyl-μ-D-thiogalactopyranoiside, “RBS” means ribosome binding site, “rpm” means revolutions per minute, “HPLC” means high performance liquid chromatography, and “GC” means gas chromatography.

### Example 1

### General Example of Genetic Modification to a Host Cell (Prophetic and Non-Specific)

This example is meant to describe a non-limiting approach to genetic modification of a selected microorganism to introduce a nucleic acid sequence of interest. Alternatives and variations are provided within this general example. The methods of this example are conducted to achieve a combination of desired genetic modifications in a selected microorganism species, such as a combination of genetic modifications selected from those shown in FIG. 1, and their functional equivalents, such as in other bacterial and other microorganism species.

A gene or other nucleic acid sequence segment of interest is identified in a particular species (such as C. necator, O. carboxidovorans, or E. coli as described above) and a nucleic acid sequence comprising that gene or segment is obtained. For clarity below the use of the term “segment of interest” below is meant to include both a gene and any other nucleic acid sequence segment of interest. One example of a method used to obtain a segment of interest is to acquire a culture of a microorganism, where that microorganism's genome includes the gene or nucleic acid sequence segment of interest.

Based on the nucleic acid sequences at the ends of or adjacent the ends of the segment of interest, 5′ and 3′ nucleic acid primers are prepared. Each primer is designed to have a sufficient overlap section that hybridizes with such ends or adjacent regions. Such primers may include enzyme recognition sites for restriction digest of transposase insertion that could be used for subsequent vector incorporation or genomic insertion. These sites are typically designed to be outward of the hybridizing overlap sections. Numerous contract services are known that prepare primer sequences to order (e.g., Integrated DNA Technologies, Coralville, Iowa USA).

Once primers are designed and prepared, polymerase chain reaction (PCR) is conducted to specifically amplify the desired segment of interest. This method results in multiple copies of the region of interest separated from the microorganism's genome. The microorganism's DNA, the primers, and a thermophilic polymerase are combined in a buffer solution with potassium and divalent cations (e.g., Mg or Mn) and with sufficient quantities of deoxynucleoside triphosphate molecules. This mixture is exposed to a standard regimen of temperature increases and decreases. However, temperatures, components, concentrations, and cycle times may vary according to the reaction according to length of the sequence to be copied, annealing temperature approximations and other factors known or readily learned through routine experimentation by one skilled in the art.

In an alternative embodiment the segment of interest may be synthesized, such as by a commercial vendor, and prepared via PCR, rather than obtaining from a microorganism or other natural source of DNA. Such sequences may be codon optimized by methods known in the art.

The nucleic acid sequences then are purified and separated, such as on an agarose gel via electrophoresis. Optionally, once the region is purified it can be validated by standard DNA sequencing methodology and may be introduced into a vector. Any of a number of vectors may be used, which generally comprise markers known to those skilled in the art, and standard methodologies are routinely employed for such introduction. Commonly used vector systems are pSMART (Lucigen, Middleton, Wis.), pET E. COLi EXPRESSION SYSTEM (Stratagene, La Jolla, Calif.), pSC-B StrataClone Vector (Stratagene, La Jolla, Calif.), pRANGER-BTB vectors (Lucigen, Middleton, Wis.), and TOPO vector (Invitrogen Corp, Carlsbad, Calif., USA). Similarly, the vector then is introduced into any of a number of host cells. Commonly used host cells are E. cloni 10G (Lucigen, Middleton, Wis.), E. cloni 10GF′ (Lucigen, Middleton, Wis.), StrataClone Competent cells (Stratagene, La Jolla, Calif.), E. coli BL21, E. coli BW25113, and E. coli K12 MG1655. Some of these vectors possess promoters, such as inducible promoters, adjacent the region into which the sequence of interest is inserted (such as into a multiple cloning site), while other vectors, such as pSMART vectors (Lucigen, Middleton, Wis.), are provided without promoters and with dephosphorylated blunt ends. The culturing of such plasmid-laden cells permits plasmid replication and thus replication of the segment of interest, which often corresponds to expression of the segment of interest.

Various vector systems comprise a selectable marker, such as an expressible gene encoding a protein needed for growth or survival under defined conditions. Common selectable markers contained on backbone vector sequences include genes that encode for one or more proteins required for antibiotic resistance as well as genes required to complement auxotrophic deficiencies or supply critical nutrients not present or available in a particular culture media. Vectors also comprise a replication system suitable for a host cell of interest.

The plasmids containing the segment of interest can then be isolated by routine methods and are available for introduction into other microorganism host cells of interest. Various methods of introduction are known in the art and can include vector introduction or genomic integration. In various alternative embodiments the DNA segment of interest may be separated from other plasmid DNA if the former will be introduced into a host cell of interest by means other than such plasmid.

While steps of the above general prophetic example involve use of plasmids, other vectors known in the art may be used instead. These include cosmids, viruses (e.g., bacteriophage, animal viruses, plant viruses), and artificial chromosomes (e.g., yeast artificial chromosomes (YAC) and bacteria artificial chromosomes (BAC)).

Host cells into which the segment of interest is introduced may be evaluated for performance as to a particular enzymatic step, and/or tolerance or bio-production of a chemical compound of interest. Selections of better performing genetically modified host cells may be made, selecting for overall performance, tolerance, or production or accumulation of the chemical of interest.

It is noted that this procedure may incorporate a nucleic acid sequence for a single gene (or other nucleic acid sequence segment of interest), or multiple genes (under control of separate promoters or a single promoter), and the procedure may be repeated to create the desired heterologous nucleic acid sequences in expression vectors, which are then supplied to a selected microorganism so as to have, for example, a desired complement of enzymatic conversion step functionality for any of the herein-disclosed metabolic pathways. However, it is noted that although many approaches rely on expression via transcription of all or part of the sequence of interest, and then translation of the transcribed mRNA to yield a polypeptide such as an enzyme, certain sequences of interest may exert an effect by means other than such expression.

The specific laboratory methods used for the above approaches are well-known in the art and may be found in various references known to those skilled in the art, such as Sambrook and Russell, Molecular Cloning: A Laboratory Manual, Third Edition 2001 (volumes 1-3), Cold Spring Harbor Laboratory Press, Cold Spring Harbor, N.Y. (hereinafter, Sambrook and Russell, 2001).

As an alternative to the above, other genetic modifications may also be practiced, such as a deletion of a nucleic acid sequence of the host cell's genome. One non-limiting method to achieve this is by use of Red/ET recombination, known to those of ordinary skill in the art and described in U.S. Pat. Nos. 6,355,412 and 6,509,156, issued to Stewart et al. and incorporated by reference herein for its teachings of this method. Material and kits for such method are available from Gene Bridges (Gene Bridges GmbH, Dresden, Germany, www.genebridges.com), and the method may proceed by following the manufacturer's instructions. Targeted deletion of genomic DNA may be practiced to alter a host cell's metabolism so as to reduce or eliminate production of undesired metabolic products. This may be used in combination with other genetic modifications such as described above in this general example. In this detailed description, reference has been made to multiple embodiments and to the accompanying drawings in which is shown by way of illustration specific exemplary embodiments in which the invention may be practiced. These embodiments are described in sufficient detail to enable those skilled in the art to practice the invention, and it is to be understood that modifications to the various disclosed embodiments may be made by a skilled artisan.

Where methods and steps described above indicate certain events occurring in certain order, those of ordinary skill in the art will recognize that the ordering of certain steps may be modified and that such modifications are in accordance with the variations of the invention. Additionally, certain steps may be performed concurrently in a parallel process when possible, as well as performed sequentially.

The embodiments, variations, sequences, and figures described herein should provide an indication of the utility and versatility of the present invention. Other embodiments that do not provide all of the features and advantages set forth herein may also be utilized, without departing from the spirit and scope of the present invention. Such modifications and variations are considered to be within the scope of the invention.

### Example 2

### Prophetic Example of 3-HP Production

An inoculum of a genetically modified microorganism that possesses enzymatic activity of numbered enzymatic conversion steps 1-12 (or 1-9 and 13, or all of 1-13) of FIG. 1 (such as may be constructed by the methods of Example 1) is provided to a culture vessel to which also is provided a liquid media comprising nutrients at concentrations sufficient for a desired bio-process culture period. This culture vessel is cultured under conditions suitable for production of 3-HP, for a period of time during which hydrogen and carbon dioxide and/or carbon monoxide, such as from a syngas source, are provided.

The final broth (comprising microorganism cells, largely ‘spent’ media and 3-HP, the latter at concentrations, in various embodiments, exceeding 1, 2, 5, 10, 30, 50, 75 or 100 grams/liter) is collected and subjected to separation and purification steps so that 3-HP is obtained in a relatively purified state. Separation and purification steps may proceed by any of a number of approaches combining various methodologies, which may include centrifugation, filtration, reduced pressure evaporation, liquid/liquid phase separation (including after forming a polyamine-3-HP complex, such as with a tertiary amine such as CAS#68814-95-9, Alamine® 336, a triC₈₋₁₀ alkyl amine (Cognis, Cincinnati, Ohio or Henkel Corp.)), membranes, distillation, and/or other methodologies recited in this patent application, incorporated herein. Principles and details of standard separation and purification steps are known in the art, for example in “Bioseparations Science and Engineering,” Roger G. Harrison et al., Oxford University Press (2003), and Membrane Separations in the Recovery of Biofuels and Biochemicals—An Update Review, Stephen A. Leeper, pp. 99-194, in Separation and Purification Technology, Norman N. Li and Joseph M. Calo, Eds., Marcel Dekker (1992), incorporated herein for such teachings. The particular combination of methodologies is selected from those described herein, and in part is based on the concentration of 3-HP and other components in the final broth.

### Example 3

### Prophetic Example of Conversion of 3-HP to Specified Downstream Chemicals

3-HP such as from Example 2 is converted to any one or more of propriolactone via a ring-forming internal esterification reaction (eliminating a water molecule), ethyl-3-HP via esterification with ethanol, malonic acid via an oxidation reaction, and 1,3-propanediol via a reduction reaction.

These conversions proceed such as by organic synthesis reactions known to those skilled in the art. Any of these conversions of 3-HP proceeds via a chemical synthesis reaction under controlled conditions to attain a high conversion rate and yield with acceptably low by-product formation.

### Example 4

### Prophetic Example of Bio-acrylic Acid Production from 3-HP

3-HP is obtained in a relatively pure state from a microbial bio-production event, such as is described in Example 2. The 3-HP is converted to acrylic acid by a dehydration reaction, such as by heating under vacuum in the presence of a catalyst. Various combinations of parameters, such as temperature, rate of change of temperature, purity of 3-HP solution derived from the microbial bio-production event, reduced pressure (and rate of change of pressure), and type and concentration of one or more catalysts, are evaluated with objectives of high conversion rate without undesired side reactions, which might, in some production scenarios, include undesired polymerization of acrylic acid. Acrylic acid so formed may be separated and purified by methods known in the art, such as those methods disclosed, supra.

### Example 5

### Alternative Prophetic Example of Bio-acrylic Acid Production from 3-HP

3-HP is obtained in a relatively pure state from a microbial bio-production event, such as is described in Example 2. The 3-HP is converted to acrylic acid by a dehydration reaction, such as by heating under vacuum in the presence of a catalyst, however under conditions favoring a controlled polymerization of acrylic acid after its formation from 3-HP. Various combinations of parameters, such as temperature, rate of change of temperature, including removal of heat generated during reaction, purity of 3-HP solution derived from the microbial bio-production event, reduced pressure (and rate of change of pressure), and type and concentration of one or more catalysts and/or exposure to light, are evaluated with objectives of high conversion rate without undesired side reactions. Acrylic acid so formed may be separated and purified by methods known in the art, such as those methods disclosed, supra.

### Example 6

### Prophetic Example of Conversions of Acrylic Acid to Downstream Products

The acrylic acid of Example 4 is further converted to one (or more) of the downstream products as described herein. For example, the conversion method is esterification with methanol to produce methyl acrylate, or other esterifications with other alcohols for other acrylate esters, amidation to produce acrylamide, adding a nitrile moiety to produce acrylonitrile. Other additions are made as desired to obtain substituted downstream compounds as described herein.

### Example 7

### Prophetic Example of Conversion of Acrylic Acid to Polyacrylic Acid

The acrylic acid of Example 4 is further converted to a polyacrylic acid by heating the acrylic acid in an aqueous solution and initiating a polymerization reaction by exposing the solution to light, and thereafter controlling the temperature and reaction rate by removing heat of the polymerization.

The specific methods and teachings of the specification, and/or cited references that are incorporated by reference, may be incorporated into the above examples. Also, production of 3-HP, or one of its downstream products such as described herein, may reach at least 1, at least 2, at least 5, at least 10, at least 20, at least 30, at least 40, and at least 50 g/liter titer in various embodiments.

### Example 8

### 3-HP Dehydration to Acrylic Acid with Acid Catalyst

3-HP stock solution was prepared as follows. A vial of β-propriolactone (Sigma-Aldrich, St. Louis, Mo., USA) was opened under a fume hood and the entire bottle contents was transferred to a new container sequentially using a 25-mL glass pipette. The vial was rinsed with 50 mL of HPLC grade water and this rinse was poured into the new container. Two additional rinses were performed and added to the new container. Additional HPLC grade water was added to the new container to reach a ratio of 50 mL water per 5 mL β-propriolactone. The new container was capped tightly and allowed to remain in the fume hood at room temperature for 72 hours. After 72 hours the contents were transferred to centrifuge tubes and centrifuged for 10 minutes at 4,000 rpm. Then the solution was filtered to remove particulates and, as needed, concentrated by use of a rotary evaporator at room temperature. Assay for concentration was conducted, and dilution to make a standard concentration stock solution was made as needed.

Approximately 15 mL of an aqueous solution comprising about 350 grams per liter of 3-HP produced above was combined in a flask with approximately 15 mL of concentrated sulfuric acid. The flask was attached to a rotary evaporator apparatus (Rotovapor Model R-210, BUCHI Labortechnik AG, Switzerland), heated in a heating bath (BUCCHI, Model B-491) to 80° C. under reduced pressure (10 to 20 mbar), and the condensate was collected below a condensing apparatus operated with chilled water as the coolant. After approximately 5 hours the condensate was collected, its volume measured, and an aliquot submitted for HPLC analysis. An aliquot of the reaction mixture in the flask also was submitted for HPLC analysis. The HPLC analysis indicated that approximately 24 grams per liter of acrylic acid was obtained in the condensate, whereas approximately 4.5 grams per liter remained in the reaction mixture of the flask. Thus, 3-HP was shown to form acrylic acid under these conditions. This example is not meant to be limiting.

### Example 9

### Prophetic Example of Conversion of Acrylic Acid to Polyacrylic Acid

Acrylic acid, such as from an example above, is further converted to a polyacrylic acid by heating the acrylic acid in an aqueous solution and initiating a free-radical polymerization reaction by exposing the solution to light, and thereafter controlling the temperature and reaction rate by removing heat of the polymerization.

Batch polymerization is utilized, wherein acrylic acid is dissolved in water at a concentration of about 50 wt %. The monomer solution is deoxygenated by bubbling nitrogen through the solution. A free-radical initiator, such as an organic peroxide, is optionally added (to assist the initiation via the light source) and the temperature is brought to about 60° C. to start polymerization.

The molecular mass and molecular mass distribution of the polymer are measured. Optionally, other polymer properties including density, viscosity, melting temperature, and glass-transition temperature are determined.

The specific methods and teachings of the specification, and/or cited references that are incorporated by reference, may be incorporated into the above examples. Also, production of 3-HP, or one of its downstream products such as described herein, may reach at least 1, at least 2, at least 5, at least 10, at least 20, at least 30, at least 40, and at least 50 g/liter titer in various embodiments.

### Example 10

### Prophetic Example of Bulk Polymerization of Acrylic Acid to Polyacrylic Acid

Acrylic acid, such as from an example above, is further converted to a polyacrylic acid by bulk polymerization. Acrylic acid monomer, monomer-soluble initiators, and neutralizing base are combined in a polymerization reactor. Polymerization is initiated, and temperature is controlled to attain a desired conversion level. Initiators are well-known in the art and include a range of organic peroxides and other compounds, such as discussed above. The acrylic acid or polyacrylic acid is at least partially neutralized with a base such as sodium hydroxide.

The molecular mass and molecular mass distribution of the polymer are measured. Optionally, other polymer properties including density, viscosity, melting temperature, and glass-transition temperature are determined.

The polyacrylic acid produced is intended for use as a superabsorbent polymer, as an absorbent for water and aqueous solutions for diapers, adult incontinence products, feminine hygiene products, and similar consumer products, as well as for possible uses in agriculture, horticulture, and other fields.

### Example 11

### Prophetic Example of Production of a Superabsorbent Polymer

Acrylic acid, such as from an example above, is further converted to a superabsorbent polyacrylic acid by solution polymerization. An aqueous solution of acrylic acid monomer (at about 25-30 wt %), initiators, neutralizing base, antioxidants, crosslinkers (such as trimethylolpropane triacrylate) and optionally other additives are combined in a polymerization reactor and polymerization is initiated. Bases that can be used for neutralization include but are not limited to sodium carbonate, sodium hydroxide, and potassium hydroxide.

The reactor contents are deoxygenated for 60 minutes. The temperature of the polymerization reaction is allowed to rise to an initial desired level. The reactor is then maintained at a desired hold temperature for a period of time necessary for the desired monomer conversion to be achieved. The resulting reaction product is in the form of a high-viscosity gel. The high-viscosity, gel-like reaction product is then processed into a film or a strand, dried and ground into particles which are screened or classified into various particle size fractions. After the polymer is dried and ground to final particulate size, it is analyzed for residual acrylic acid and other chemicals, extractable centrifuge capacity, shear modulus, and absorption under load. Other polymer properties may be measured, including molecular mass, molecular mass distribution, density, viscosity, melting temperature, and glass-transition temperature. Surface treatments may be performed by adding a cross-linking co-monomer to the surface of the polymer particles.

The polyacrylic acid produced is intended for use as a superabsorbent polymer, as an absorbent for water and aqueous solutions for diapers, adult incontinence products, feminine hygiene products, and similar consumer products, as well as for possible uses in agriculture, horticulture, and other fields.

### Example 12

### Alternative Prophetic Example of Production of a Superabsorbent Polymer

Acrylic acid, such produced from an example above, is further converted to a superabsorbent polyacrylic acid by suspension polymerization. An aqueous phase comprising water, acrylic acid monomer, and neutralizing base is combined with an an oil phase comprising an inert hydrophobic liquid and optionally a suspending agent is further provided. The aqueous phase and the oil phase are contacted under conditions (including a temperature of about 75° C.) such that fine monomer droplets are formed. Polymerization is initiated, and the polymerized microparticles of polyacrylic acid are recovered from the suspension using a centrifuge.

The polyacrylic acid is then dried and ground into particles which are screened or classified into various particle size fractions. After the polymer is dried and ground to final particulate size, it is analyzed for residual acrylic acid and other chemicals, extractable centrifuge capacity, shear modulus, and absorption under load. Other polymer properties may be measured, including molecular mass, molecular mass distribution, density, viscosity, melting temperature, and glass-transition temperature.

The polyacrylic acid produced is intended for use as a superabsorbent polymer, as an absorbent for water and aqueous solutions for diapers, adult incontinence products, feminine hygiene products, and similar consumer products, as well as for possible uses in agriculture, horticulture, and other fields.

### Example 13

### Prophetic Example of Conversion of Acrylic Acid to Methyl Acrylate

Acrylic acid, such as produced from an example above, is converted to methyl acrylate by direct, catalyzed esterification. Acrylic acid is contacted with methanol, and the mixture is heated to about 50° C. in the presence of an esterification catalyst. Water formed during esterification is removed from the reaction mixture by distillation. The progress of the esterification reaction is monitored by measuring the concentration of acrylic acid and/or methanol in the mixture.

Reactive with other monomers and imparting strength and durability to acrylic co-polymers, methyl acrylate is a useful monomer for coatings for leather, paper, floor coverings and textiles. Resins containing methyl acrylate can be formulated as elastomers, adhesives, thickeners, amphoteric surfactants, fibers and plastics. Methyl Acrylate is also used in production of monomers used to make water treatment materials and in chemical synthesis.

### Example 14

### Prophetic Example of Conversion of Acrylic Acid to Ethyl Acrylate

Acrylic acid, such as produced from an example above, is converted to ethyl acrylate by direct, catalyzed esterification. Acrylic acid is contacted with ethanol, and the mixture is heated to about 75° C. in the presence of an esterification catalyst. Water formed during esterification is removed from the reaction mixture by distillation. The progress of the esterification reaction is monitored by measuring the concentration of acrylic acid and/or ethanol in the mixture.

Ethyl acrylate is used in the production of homopolymers and co-polymers for use in textiles, adhesives and sealants. Ethyl acrylate is also used in the production of co-polymers, for example acrylic acid and its salts, esters, amides, methacrylates, acrylonitrile, maleates, vinyl acetate, vinyl chloride, vinylidene chloride, styrene, butadiene and unsaturated polyesters. In addition, ethyl acrylate is used in chemical synthesis.

### Example 15

### Prophetic Example of Conversion of Acrylic Acid to Butyl Acrylate

Acrylic acid, such as produced from an example above, is converted to butyl acrylate by direct, catalyzed esterification. Acrylic acid is contacted with 1-butanol, and the mixture is heated to about 100° C. in the presence of an esterification catalyst. Water formed during esterification is removed from the reaction mixture by distillation. The progress of the esterification reaction is monitored by measuring the concentration of acrylic acid and/or ethanol in the mixture.

Butyl acrylate is used in the production of homopolymers and co-polymers for use in water-based industrial and architectural paints, enamels, adhesives, caulks and sealants, and textile finishes, utilizing homopolymers and co-polymers with methacrylates, acrylonitrile, maleates, vinyl acetate, vinyl chloride, vinylidene chloride, styrene, butadiene or unsaturated polyesters.

### Example 16

### Prophetic Example of Conversion of Acrylic Acid to Ethylhexyl Acrylate

Acrylic acid, such as produced from an example above, is converted to ethylhexyl acrylate by direct, catalyzed esterification. Acrylic acid is contacted with 2-ethyl-1-hexanol, and the mixture is heated to about 120° C. in the presence of an esterification catalyst. Water formed during esterification is removed from the reaction mixture by distillation. The progress of the esterification reaction is monitored by measuring the concentration of acrylic acid and/or ethanol in the mixture.

Ethylhexyl acrylate is used in the production of homopolymers and co-polymers for caulks, coatings and pressure-sensitive adhesives, paints, leather finishing, and textile and paper coatings.

### Example 17

### Prophetic Example of Conversion of Acrylates to End Products, Including Consumer Products

One or more acrylates as provided in Examples 13-16 is further converted to one or more of adhesives, surface coatings, water-based coatings, paints, inks, leather finishes, paper coatings, film coatings, plasticizers, or precursors for flocculants. Such conversions to end products employ methods known in the art.

### Example 18

### Prophetic Example of Acrylic-Based Paint Manufacture

An aqueous dispersion comprising at least one particulate water-insoluble copolymer that includes one or more of acrylic acid, ethyl acrylate, methyl acrylate, 2-ethylhexyl acrylate, butyl acrylate, lauryl acrylate or other copolymer obtained from acrylic acid converted from 3-HP microbially produced, as described elsewhere herein, is obtained by mixing such components together under sufficient agitation to form a stable dispersion of the copolymers. The copolymers have an average molecular weight that is at least 50,000, with the copolymer particles having diameters in the range of 0.5 to 3.0 microns, Other components in the aqueous dispersion may include pigment, filler (e.g., calcium carbonate, aluminum silicate), solvent (e.g., acetone, benzol, alcohols, etc., although these are not found in certain no VOC paints), thickener, and additional additives depending on the conditions, applications, intended surfaces, etc.

In variations of such acrylic-based paints, co-polymers in addition to the acrylic-based polymers may be added. Such other co-polymers may include, but are not limited to vinyl acetate, vinyl fluoride, vinylidene chloride, methacrylic acid, itaconic acid, maleic acid, and styrene.

### Example 19

### Prophetic Example of Conversion of 3-HP to 1,3-Propanediol

Acrylic acid, such as produced from an example above, is converted to 1,3-propanediol. 3-HP is hydrogenated in the presence of an unsupported ruthenium catalyst, in a liquid phase, to prepare 1,3-propanediol. The liquid phase includes water and cyclohexane. The hydrogenation is carried out continuously in a stirred tank reactor at a temperature of about 150° C. and a pressure of about 1000 psi. The progress of hydrogenation is monitored by measuring the concentration of 3-HP and/or hydrogen in the reactor.

### Example 20

### Prophetic Example of Conversion of 3-HP to Malonic Acid

Acrylic acid, such as that provided in Example 22, is converted to malonic acid by catalytic oxidation of 3-HP by a supported catalyst comprising Rh. The catalytic oxidation is carried out in a fixed-bed reactor operated in a trickle-bed procedure. In the trickle-bed procedure the aqueous phase comprising the 3-HP starting material, as well as the oxidation products of the same and means for the adjustment of pH, and oxygen or an oxygen-containing gas can be conducted in counterflow. In order to achieve a sufficiently short reaction time, the conversion is carried out at a pH of about 8. The oxidation is carried out at a temperature of about 40° C. Malonic acid is obtained in nearly quantitative yields.

### Example 21

### Construction of C. necator Strains for Evaluation (Prophetic)

Part 1: Gene Deletions

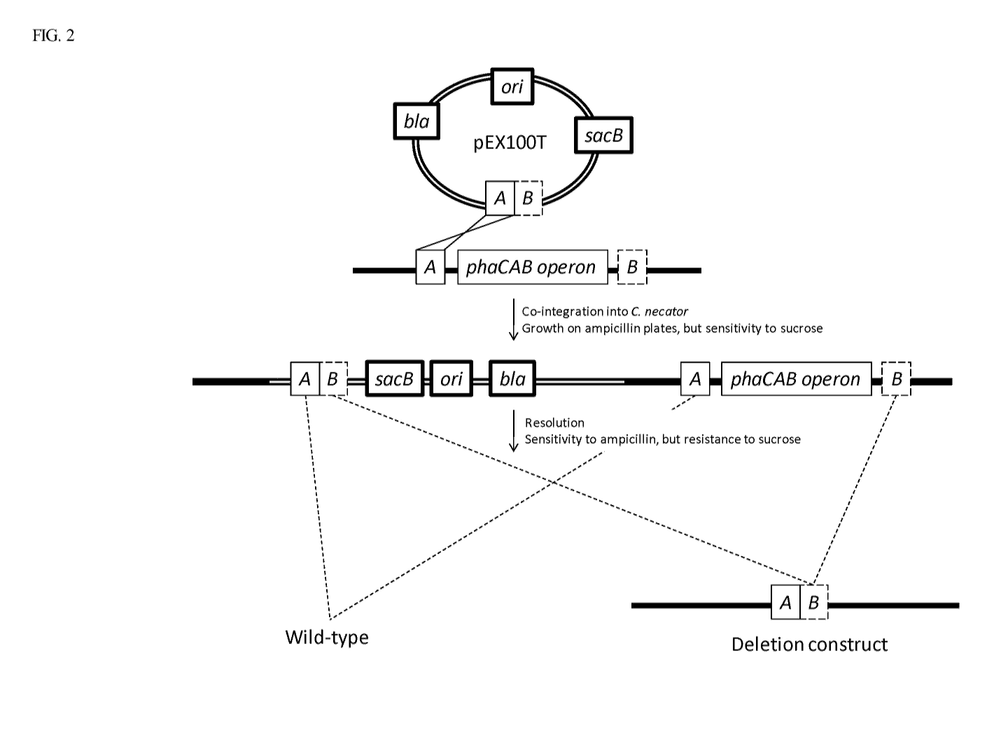

The homologous recombination method using integration of counterselectable suicide vectors, is employed for gene deletion in C. necator strains. This method is known to those of ordinary skill in the art. The method integrates a target sequence including both a selectable marker and counterselectable marker via homologous recombination performed by host recombination machinery. Integrants are selected via the selectable marker, following the approach depicted in FIG. 2. The markers are then removed by counterselection and 2 genotypes are distinguished by screening via PCR, one would be wild type, the second the desired gene deletion, integration or replacement.

Specific gene deletions in C. necator are constructed by creating counterselectable suicide vectors that will delete the genes or operons. These vectors are constructed by gene synthesis or via cloning using overlapping PCR.

Table 6 below list the desired genes and or operons that are deleted singly and in combination in C. necator strains that produce free fatty acids and fatty acid derived products including 3-HP.

TABLE 6

| Gene/Operon Name/E.C. # | Function |
|---|---|
| phaCAB | Polyhydroxybutyrate |
| | formation |
| E.C. 1.2.1.18 | Malonate semialdehyde |
| | degradation |
| E.C. 1.1.1.59 betA4 | 3-hydoxypropionate |
| | dehydrogenase |

Part 2: Construction of Plasmids for Gene Overexpression, and/or Chromosomal Integration

In addition to the construction of gene deletions and integrations in C. necator, replicating plasmids may be used to introduce genetic modifications into C. necator strains including those that enable the overexpression of desired genes and the increase in desired enzyme functions. Cloning and expression of genes can be performed in numerous plasmids. For example small broad host range vectors may be used for expression such as pBT-3 (see U.S. Patent Publication No. 2007/0059768, published Mar. 15, 2007, and incorporated by reference for its teachings of the construction and use of these vectors.) In addition to overexpressing the genes and enzymes listed in Table 1 on plasmids enabling the production of 3-HP in C. necator, so as to have sufficient enzymatic conversion through step 9 and/or through step 11, the production of 3-HP requires the expression of a 3-HP dehydrogenase (step 13, identified as a 3-hydroxy acid dehydrogenase) and/or malonyl-coA reductase (step 12, which may be a bifunctional malonyl-CoA reductase or a monofunctional malonyl-CoA reductase combined with a 3-HP dehydrogenase). Expression of these genes or improved mutants or homologous alternative thereof may be expressed in C. necator on plasmids. In addition any gene listed in Table 1 is integrated into the chromosome(s) of C. necator using standard methods, such as the GeneBridges homologous recombination method referenced herein. For example, an NADPH-dependent 3-hydroxy acid dehydrogenase could replace an NADP-dependent 3-hydroxy acid dehydrogenase, such as the NADH-dependent 3-HP dehydrogenase noted above in Table 6, so as to obtain more effective overall 3-HP production. In general, such modifications may be made to delete NADH-dependent 3-HP dehydrogenases and overexpress NADPH-dependent 3-hydroxy acid dehydrogenases, particularly those having an elevated 3-HP dehydrogenase activity and specificity.

Part 3: Construction of Strains

Any combination of gene deletions and gene overexpressions described above may be incorporated into a single C. necator strain for the production of 3-HP.

TABLE 1

| | | | Relevant SEQ ID | | |
|---|---|---|---|---|---|
| Step | EC | Representative gene of stated species, and | NOs. for rep gene | Substrate in Pathway for | Product in Pathway for this |
| No. | Number | enzyme name | and its product | this Reaction | Reaction |
| 1 | 5.3.1.9 | pgi of E. coli, phosphoglucose isomerase | 001, 002 | Fructose-6-phosphate | Glucose-6-phosphate |
| 2 | 5.5.1.4 | ino-1 of S. cerevisiae, inositol-1- | 003, 004 | Glucose-6-phosphate | Myo-inositol-3-phosphate |
| | | phosphate synthase | | | |
| 3 | 3.1.3.25 | suhB of E. coli, inositol | 005, 006 | Myo-inositol-3-phosphate | Myo-inositol |
| | | monophosphatase | | | |
| 4 | | iolG of B. subtilis, myo-inositol | 007, 008 | Myo-inositol | 2-keto-myo-inositol |
| | | dehydrogenase | | | |
| 5 | | iolE of B. subtilis, myo-inosose-2- | 009, 010 | 2-keto-myo-inositol | D-2,3-diketo-4-deoxy-epi- |
| | | dehydratase | | | inositol |
| 6 | 1.1.1.18 | iolD of B. subtilis, diketo deoxyinositol | 011, 012 | D-2,3-diketo-4-deoxy-epi- | 5-deoxy D-gluconate (5- |
| | | hydrolase (or inositol-2 dehydrogenase) | | inositol | deoxy-D-glucoronic acid) |
| 7 | | iolB of B. subtilis, an isomerase | 013, 014 | 5-deoxy D-gluconate | 5-dehydro-2-deoxy-D- |
| | | | | | gluconate |
| 8 | | iolC of B. subtilis, a kinase | 015, 016 | 5-dehydro-2-deoxy-D- | 5-dehydro-2-deoxy-D- |
| | | | | gluconate | gluconate-6-phosphate |
| 9 | | iolJ of B. subtilis, an aldolase | 017, 018 | 5-dehydro-2-deoxy-D- | Malonate semialdehyde & |
| | | | | gluconate-6-phosphate | Dihydoxyacetone phosphate |
| 10 | 1.2.1.21 | aldA of E. coli, aldehyde dehydrogenase | 019, 020 | Malonate Semialdehyde | Malonate |
| 11 | | matB of Rhizobium leguminosum, | 021, 022 | Malonate | Malonyl-CoA |
| | | malonyl-CoA synthetase | | | |
| 12 | 1.2.1.75 | Malonyl-CoA reductase of Chloroflexus | 023, 024 | Malonyl-CoA | Malonate semialdehyde |
| | 1.1.1.298 | aurantiacus (exemplifying a bifunctional | | Malonate Semialdehyde | 3-hydroxypropionic acid (3- |
| | | mcr) | | | HP) |
| 13 | 1.1.1.276 | ydfG of E. coli, 3-hydroxy acid | 025, 026 | Malonate semialdehyde | 3-hydroxypropionic acid (3- |
| | | dehydrogenase (or L-allo-threonine | | | HP) |
| | | dehydrogenase) | | | |

TABLE 2

| | | Representative gene of | Primary | | O. carboxidovorans Additional |
|---|---|---|---|---|---|
| Step | EC | stated species, and | Candidate Protein | C. necator Additional Candidate NCBI | Candidate NCBI Reference |
| No. | Number | enzyme name | SEQ ID NOs. | Reference Sequence (version) | Sequence (version) |
| 1 | 5.3.1.9 | pgi of E. coli, | 002 | YP_726002.1; YP_841019.1 | YP_002288070.1 |
| | | phosphoglucose | | | |
| | | isomerase | | | |
| 2 | 5.5.1.4 | ino-1 of S. cerevisiae, | 004 | | |
| | | inositol-1-phosphate | | | |
| | | synthase | | | |
| 3 | 3.1.3.25 | suhB of E. coli, inositol | 006 | YP_725723.1 | YP_002288070.1; |
| | | monophosphatase | | | YP_002290224.1; |
| | | | | | YP_002287949.1; |
| | | | | | YP_002290089.1 |
| 4 | | iolG of B. subtilis, myo- | 008 | | YP_002288336.1 |
| | | inositol dehydrogenase | | | |
| 5 | | iolE of B. subtilis, myo- | 010 | | |
| | | inosose-2-dehydratase | | | |
| 6 | 1.1.1.18 | iolD of B. subtilis, | 012 | YP_726696.1; YP_728024.1; | YP_002288287.1; |
| | | diketo deoxyinositol | | YP_725545.1; YP_841382.1; | YP_002289539.1; |
| | | hydrolase (or inositol-2 | | YP_841964.1; YP_728478.1; | YP_002289720.1; |
| | | dehydrogenase) | | YP_841227.1; YP_725622.1; | YP_002289528.1 |
| | | | | YP_728897.1 | |
| 7 | | iolB of B. subtilis, an | 014 | | |
| | | isomerase | | | |
| 8 | | iolC of B. subtilis, a | 016 | YP_841324.1; YP_840732.1 | YP_002287538.1; |
| | | kinase | | | YP_002290514.1 |
| 9 | | iolJ of B. subtilis, an | 018 | YP_725085.1; YP_840903.1; | |
| | | aldolase | | NP_943051.1 | |
| 10 | 1.2.1.21 | aldA of E. coli, aldehyde | 020 | YP_729138.1; YP_841054.1; | YP_002287993.1; |
| | | dehydrogenase | | YP_726388.1; YP_841569.1; | YP_002288100.1; |
| | | | | YP_725996.1; YP_841247.1; | YP_002290043.1; |
| | | | | YP_841263.1; YP_841051.1; | YP_002289863.1; |
| | | | | YP_728899.1; YP_841451.1; | YP_002290472.1; |
| | | | | YP_841391.1; YP_728709.1; | YP_002289734.1 |
| | | | | YP_725261.1; YP_728990.1; | |
| | | | | YP_841472.1; YP_840700.1; | |
| | | | | YP_841459.1; YP_840712.1; | |
| | | | | YP_724792.1; YP_728089.1; | |
| | | | | YP_841956.1; YP_725623.1; | |
| | | | | YP_727782.1; YP_728056.1; | |
| | | | | YP_726253.1; YP_724752.1; | |
| | | | | YP_728301.1; YP_841429.1; | |
| | | | | YP_840877.1; YP_728487.1 | |
| 11 | | matB of Rhizobium | 022 | YP_727422.1; YP_726717.1; | YP_002287299.1; |
| | | leguminosum, malonyl- | | YP_727730.1; YP_727253.1; | YP_002289782.1; |
| | | CoA synthetase | | YP_727941.1; YP_727195.1; | YP_002290346.1; |
| | | | | YP_840892.1; YP_728876.1; | YP_002289754.1; |
| | | | | YP_726440.1; YP_840759.1; | YP_002288303.1; |
| | | | | YP_729067.1; YP_728340.1; | YP_002287342.1; |
| | | | | YP_728839.1; YP_728806.1; | YP_002290305.1 |
| | | | | YP_727241.1; YP_725386.1; | |
| | | | | YP_727788.1; YP_841208.1; | |
| | | | | YP_725803.1; YP_840784.1; | |
| | | | | YP_725354.1; YP_841224.1; | |
| | | | | YP_725381.1; YP_725915.1; | |
| | | | | NP_943034.1; YP_726544.1; | |
| | | | | YP_726914.1; YP_726210.1; | |
| | | | | YP_841430.1; YP_728915.1; | |
| | | | | YP_840623.1; YP_727391.1; | |
| | | | | YP_726193.1; YP_728858.1; | |
| | | | | YP_841248.1; YP_840917.1; | |
| | | | | YP_725738.1; YP_842034.1; | |
| | | | | YP_725706.1; YP_726019.1; | |
| | | | | NP_943033.1; YP_840669.1; | |
| | | | | YP_726615.1; YP_841612.1; | |
| | | | | YP_840855.1; YP_726980.1; | |
| | | | | YP_728991.1; YP_841198.1; | |
| | | | | YP_841202.1; YP_841202.1; | |
| | | | | YP_726919.1; YP_841200.1; | |
| | | | | YP_725992.1; YP_841201.1; | |
| | | | | YP_726116.1; YP_724804.1 | |

TABLE 4

| Amino Acid | Relationships | MW | TLC | SLC | DNA codons |
|---|---|---|---|---|---|
| Alanine | N, Ali | 89 | Ala | A | GCT, GCC, GCA, GCG |
| Proline | N | 115 | Pro | P | CCT, CCC, CCA, CCG |
| Valine | N, Ali | 117 | Val | V | GTT, GTC, GTA, GTG |
| Leucine | N, Ali | 131 | Leu | L | CTT, CTC, CTA, CTG, TTA, |
| | | | | | TTG |
| Isoleucine | N, Ali | 131 | Ile | I | ATT, ATC, ATA |
| Methionine | N | 149 | Met | M | ATG |
| Phenylalanine | N, Aro | 165 | Phe | F | TTT, TTC |
| Tryptophan | N | 204 | Trp | W | TGG |
| Glycine | PU | 75 | Gly | G | GGT, GGC, GGA, GGG |
| Serine | PU | 105 | Ser | S | TCT, TCC, TCA, TCG, AGT, |
| | | | | | AGC |
| Threonine | PU | 119 | Thr | T | ACT, ACC, ACA, ACG |
| Asparagine | PU, Ami | 132 | Asn | N | AAT, AAC |
| Glutamine | PU, Ami | 146 | Gln | Q | CAA, CAG |
| Cysteine | PU | 121 | Cys | C | TGT, TGC |
| Aspartic acid | NEG, A | 133 | Asp | D | GAT, GAC |
| Glutamic acid | NEG, A | 147 | Glu | E | GAA, GAG |
| Arginine | POS, B | 174 | Arg | R | CGT, CGC, CGA, CGG, |
| | | | | | AGA, AGG |
| Lysine | POS, B | 146 | Lys | K | AAA, AAG |
| Histidine | POS | 155 | His | H | CAT, CAC |
| Tyrosine | Aro | 181 | Tyr | Y | TAT, TAC |
| Stop Codons | | | | Stop | TAA, TAG, TGA |

Legend:
MW = molecular weight, rounded off.
TLC = three-letter code.
SLC = single-letter code.
As to side groups and other related properties:
A = acidic;
B = basic;
Ali = aliphatic;
Ami = amine;
Aro = aromatic;
N = nonpolar;
PU = polar uncharged;
NEG = negatively charged;
POS = positively charged.

What is claimed is:

1. A method for producing 3-hydroxypropionic acid (3-HP) comprising:
i) combining hydrogen, a carbon source selected from carbon monoxide and carbon dioxide, and a culture of microorganism cells, wherein
a) said microorganism cells are genetically transformed to introduce or increase one or more enzymatic activities for conversion of the carbon source to malonate semialdehyde, wherein said enzymatic activities are selected from the group consisting of phosphoglucose isomerase, inositol-1-phosphate synthase, inositol deoxy-D-gluconate isomerase, 5-dehydro-2-deoxygluconokinase, and deoxyphophogluconate aldolase; and
b1) said microorganism cells are genetically transformed to introduce or increase enzymatic activity for conversion of malonate semialdehyde to 3-HP, and/or
b2) said microorganism cells are capable of producing 3-HP at a rate of at least 1 g/L/hr in the absence of genetic modification for conversion of malonate semialdehyde to 3-HP; and
ii) maintaining the combined hydrogen, carbon source, and microorganism cells for a suitable time and under conditions sufficient to produce malonate semialdehyde and convert the malonate semialdehyde to 3-HP.

2. A method of producing acrylic acid comprising:
i) producing 3-HP according to the method of claim 1; and
ii) converting said 3-HP to acrylic acid.

3. A method of producing an acrylic acid-based product comprising;
i) producing acrylic acid according to the method of claim 2; and
ii) converting said acrylic acid into an acrylic acid-based product.

4. The method of claim 1, wherein said carbon source has a ratio of carbon-14 to carbon-12 of about 1.0×10⁻¹⁴ or greater.

5. The method of claim 1, wherein said carbon source has a percentage of petroleum origin selected from less than about 50%, less than about 40%, less than about 30%, less than about 20%, less than about 10%, less than about 5%, less than about 1%, or essentially free of petroleum origin.

6. The method of claim 1, wherein said carbon source has an amount of glucose, sucrose, fructose, dextrose, lactose, glycerol, and/or combinations thereof that is selected from the group consisting of less than about 50%, less than about 40%, less than about 30%, less than about 20%, less than about 10%, less than about 5%, and less than about 1% by weight.

7. The method of claim 1, wherein said cell culture comprises an inhibitor of fatty acid synthesis or said microorganism is genetically modified for reduced enzymatic activity in one or more of the microorganism's fatty acid synthesis pathways.

8. The method of claim 7, wherein said inhibitor of fatty acid synthesis is selected from the group consisting of thiolactomycin, triclosan, cerulenin, thienodiazaborine, isoniazid, and analogs thereof.

9. The method of claim 1, wherein the percentage of carbon source converted to 3-HP is selected from greater than 25%, greater than 35%, greater than 45%, greater than 55%, greater than 65%, greater than 75%, greater than 85%, and greater than 95%.

10. The method of claim 1, wherein the volumetric productivity for 3-HP is selected from at least 1/g/L/hr and at least 2/g/L/hr.

11. A method for producing malonate semialdehyde comprising:
a) combining hydrogen, a carbon source selected from carbon monoxide and carbon dioxide, and a culture of microorganism cells, wherein said microorganism cells comprise at least two genetic modifications to introduce or increase one or more enzymatic activities selected from the group consisting of phosphoglucose isomerase, inositol-1-phosphate synthase, inositol monophosphatase, myo-inositol dehydrogenase, myo-inosose-2-dehydratase, inositol 2-dehydrogenase, deoxy-D-gluconate isomerase, 5-dehydro-2-deoxygluconokinase, and deoxyphophogluconate aldolase;
b) maintaining the combined hydrogen, carbon source, and microorganism cells for a suitable time and under conditions sufficient to convert the carbon source to malonate semialdehyde.

12. A method for producing 3-HP comprising:
i) producing malonate semialdehyde according to the method of claim 11; and
ii) maintaining the microorganism cells for a suitable time and under conditions sufficient to convert the malonate semialdehyde to 3-HP;
wherein the microorganism cells further comprise a protein that converts malonate semialdehyde to 3-HP.

13. The method of claim 12, wherein the protein has 3-hydroxy acid dehydrogenase activity.

14. The method of claim 13, wherein the protein is E. coli YdfG.

15. A method for producing acrylic acid comprising:
i) producing 3-HP according to the method of claim 12; and
ii) dehydrating said 3-HP to produce acrylic acid.

16. A method for producing an acrylic-acid-based product comprising:
i) producing acrylic acid according to the method of claim 15; and
ii) processing the acrylic acid into an acrylic-acid-based product.

17. A genetically modified microorganism for the production of 3-HP, wherein said microorganism comprises at least one heterologous nucleic acid molecule selected from the groups of nucleic acid molecules encoding
a. phosphoglucose isomerase, inositol-1-phosphate synthase, inositol monophosphatase, myo-inositol dehydrogenase, myo-inosose-2-dehydratase, inositol 2-dehydrogenase, deoxy-D-gluconate isomerase, 5-dehydro-2-deoxygluconate kinase, 5-dehydro-2-deoxyphophogluconate aldolase; or
b. 3-hydroxy acid dehydrogenase.

18. The genetically modified microorganism of claim 17, wherein the number of genetic modifications is selected from at least two, at least three, at least four, at least five, at least six, at least seven, at least eight, at least nine, and at least ten enzymatic activities.

19. The genetically modified microorganism of claim 17, wherein said microorganism is selected from the group consisting of chemolithotrophic bacteria.

20. The genetically modified microorganism of claim 17, wherein said microorganism is selected from the group consisting Oligotropha carboxidovorans, Cupriavidus necator, and strain H16 of Cupriavidus necator.

21. The genetically modified microorganism of claim 17, wherein the heterologous nucleic acid molecule is selected from the group:
i) phosphoglucose isomerase encoded by the pgi gene of E. coli;
ii) inositol-1-phosphate synthase encoded by the ino-1 gene of S. cerevisiae;
iii) inositol monophosphatase encoded by the subB gene of E. coli;
iv) myo-inositol dehydrogenase encoded by the iolG gene of B. subtilis;
v) myo-inosose-2-dehydratase encoded by the iolE gene of B. subtilis;
vi) inositol 2-dehydrogenase encoded by the iolD gene of B. subtilis;
vii) deoxy-D-gluconate isomerase encoded by the iolB gene of B. subtilis;
viii) 5-dehydro-2-deoxygluconokinase encoded by the iolC gene of B. subtilis;
ix) 5-dehydro-2-deoxyphophogluconate aldolase encoded by the iolJ gene of B. subtilis; or
x) 3-hydroxy acid dehydrogenase encoded by the ydfG gene of E. coli.

22. The genetically modified microorganism of claim 17 comprising at least one genetic modification to introduce or increase one or more enzymatic activities provided by amino acid sequences having at least 50%, 60%, 70%, 80%, 85%, 90%, 92%, 95%, 96%, 97%, 98% or 99% sequence identity to one or more amino acid sequences selected from the group consisting of SEQ ID NO:002, SEQ ID NO:004, SEQ ID NO:006, SEQ ID NO:008, SEQ ID NO:010, SEQ ID NO:012, SEQ ID NO:014, SEQ ID NO:016, SEQ ID NO:018, SEQ ID NO:020, and conservatively modified variants thereof.

23. The genetically modified microorganism of claim 17 comprising at least one genetic modification provided by a polynucleotide comprising a nucleic acid sequence having at least 50%, 60%, 70%, 80%, 85%, 90%, 92%, 95%, 96%, 97%, 98% or 99% sequence identity to one or more nucleic acid sequences from the group consisting of SEQ ID NO:001, SEQ ID NO:003, SEQ ID NO:005, SEQ ID NO:007, SEQ ID NO:009, SEQ ID NO:011, SEQ ID NO:013, SEQ ID NO:015, SEQ ID NO:017, SEQ ID NO:019, and conservatively modified variants thereof.

24. A culture system comprising
(i) a population of genetically modified microorganisms of any one of claims 17 to 23; and (ii) a media comprising nutrients for said population.

25. A method of making a genetically modified microorganism according to claim 17 comprising providing to a microorganism at least one genetic modification to introduce or increase one or more enzymatic activities provided by amino acid sequences having at least 50%, 60%, 70%, 80%, 85%, 90%, 92%, 95%, 96%, 97%, 98% or 99% sequence identity to one or more amino acid sequences selected from the group consisting of SEQ ID NO:002, SEQ ID NO:004, SEQ ID NO:006, SEQ ID NO:008, SEQ ID NO:010, SEQ ID NO:012, SEQ ID NO:014, SEQ ID NO:016, SEQ ID NO:018, SEQ ID NO:020, and conservatively modified variants thereof.

26. A method of making a genetically modified microorganism according to claim 17, comprising providing to a selected microorganism at least one genetic modification comprising through a polynucleotide comprising a nucleic acid sequence having at least 50%, 60%, 70%, 80%, 85%, 90%, 92%, 95%, 96%, 97%, 98% or 99% sequence identity to one or more nucleic acid sequences from the group consisting of SEQ ID NO:001, SEQ ID NO:003, SEQ ID NO:005, SEQ ID NO:007, SEQ ID NO:009, SEQ ID NO:011, SEQ ID NO:013, SEQ ID NO:015, SEQ ID NO:017, SEQ ID NO:019, and conservatively modified variants thereof.

27. The method of claim 1, wherein said genetically modified microorganism is further modified to decrease activity in an enzyme selected from the group consisting of lactate dehydrogenase, phophate acetyltransferase, pyruvate oxidase, pyruvate-formate lyase, and combinations thereof.

28. The method of claim 1, further comprising separating and/or purifying 3-HP from said cell culture by extraction of 3-HP from said culture in the presence of a tertiary amine.

29. The method of claim 1, wherein said 3-HP is produced at a specific productivity of at least 0.005 g/gDCW-hr, 0.05 g/gDCW-hr, 1 g/gDCW-hr, or at least 2 g/gDCW-hr, or at a volumetric productivity of at least 0.50 grams per liter per hour.

30. The method of claim 3, wherein said consumer product is selected from the group consisting of diapers, carpet, paint, and adhesives.

31. The method of claim 30, wherein said consumer product is diapers.

32. Biologically-produced 3-hydroxypropionic acid, wherein said 3-hydroxypropionic acid is produced according to any one of claims 1-31.

33. The 3-hydroxypropionic acid according to claim 32, wherein said 3-hydroxypropionic acid is essentially free of chemical catalyst.

34. The 3-hydroxypropionic acid according to claim 33, wherein said chemical catalyst is a molybdenum and/or vanadium based catalyst.

35. The 3-hydroxypropionic acid according to claim 32, wherein said 3-hydroxypropionic acid has a ratio of carbon-14 to carbon-12 of about 1.0×10⁻¹⁴ or greater.

36. The 3-hydroxypropionic acid according to claim 32, wherein said 3-hydroxypropionic acid contains less than about 10% carbon derived from petroleum.

37. The 3-hydroxypropionic acid according to claim 32, wherein said 3-hydroxypropionic acid contains a residual amount of organic material related to its method of production.

38. The 3-hydroxypropionic acid according to claim 37, wherein said 3-hydroxypropionic acid contains a residual amount of organic material in an amount between 1 and 1,000 parts per million of said 3-hydroxypropionic acid.

39. Acrylic acid produced from 3-hydroxypropionic acid according to one of claims 32-38.

40. A polymer produced with acrylic acid according to claim 39.

41. A consumer product produced with acrylic acid according to claim 39.

42. The consumer product of claim 41, wherein said consumer product is selected from diapers, carpet, paint, and adhesives.

43. The consumer product of claim 42, wherein said consumer product is diapers.

44. The method of claim 1, additionally comprising converting the 3-HP molecule(s) to a chemical selected from the group consisting of acrylic acid, acrylamide, acrylonitrile, propiolactone, ethyl 3-HP, malonic acid, 1,3-propanediol, methyl acrylate, ethyl acrylate, n-butyl acrylate, hydroxypropyl acrylate, hydroxyethyl acrylate, isobutyl acrylate, and 2-ethylhexyl acrylate.

45. The method of claim 1, additionally comprising converting the 3-HP molecule(s) to acrylic acid via a dehydration reaction.

46. The method of claim 45, additionally comprising converting the acrylic acid to a polyacrylic acid via a polymerization reaction.

47. The method of claim 1, additionally comprising converting the 3-HP molecule(s) to methyl acrylate via dehydration and esterification.

48. The method of claim 1, additionally comprising converting the 3-HP molecule(s) to acrylamide via dehydration and amidation.

49. The method of claim 1, additionally comprising converting the 3-HP molecule(s) to acrylonitrile.

50. The method of claim 1, additionally comprising converting the 3-HP molecule(s) to propriolactone.

51. The method of claim 1, additionally comprising converting the 3-HP molecule(s) to ethyl 3-HP.

52. The method of claim 1, additionally comprising converting the 3-HP molecule(s) to malonic acid.

53. The method of claim 1, additionally comprising converting the 3-HP molecule(s) to 1,3-propanediol.

54. The method of claim 1, additionally comprising forming a polymerized-3-HP (poly-3HP) polymer.

55. The method of claim 1, additionally comprising forming a polymer from 3-HP (such as poly-3HP), acrylic acid (such as polyacrylic acid), and/or at least one downstream compound of 3-HP.

56. The method of claim 55 wherein at least one downstream compound of 3-HP is selected from the group consisting of acrylic acid, acrylamide, acrylonitrile, propiolactone, ethyl 3-HP, malonic acid, 1,3-propanediol, methyl acrylate, ethyl acrylate, n-butyl acrylate, hydroxypropyl acrylate, hydroxyethyl acrylate, isobutyl acrylate, and 2-ethylhexyl acrylate.
